SEQUENCE LISTING (298 sequences)

SEQ ID NO: 1 - DNA, 2022 bp, Fusarium graminearum
  source 1..2022: mol_type = other DNA
atgggcagcagccatcatcatcatcatcacagcagcggcctggtgccgcgcggcagccatatgggtaccgcttctgctccaattggttctgctatttccagaaacaattgggctgttacttgtgattctgctcaatctggtaatgaatgtaacaaggctattgatggtaacaaggatactttttggcatactttctacggtgctaacggagatccaaaaccacctcacacttacactatcgatatgaagactactcaaaacgttaatggtttgtctatgttgcctagacaagatggtaaccaaaatggttggattggtagacatgaagtttacttgtcttctgatggtactaattggggttctcctgttgcttccggttcttggtttgctgattctactactaaatactctaacttcgagactagaccagctagatatgttagattggttgctgttactgaagctaatggtcaaccttggacttctattgctgagattaacgtttttcaggcttcttcttacactgctccacaacctggtttgggtagatggggtccaactattgatttgccaattgttcctgctgctgctgctattgagcctacttctggtagagttttgatgtggtcttcttatagaaatgatgcttttggtggttctccaggtggtattactttgacttcttcttgggacccatctactggtattgtttctgatagaactgttactgttactaagcatgatatgttctgtccaggtatttctatggatggtaatggtcaaattgttgttactggtggtaacgatgctaagaaaacttctttgtacgattcttcttctgattcttggattccaggtcctgatatgcaagttgctagaggttatcaatcttctgctactatgtctgatggtagagtttttactattggtggttcttggtctggtggtgttttcgaaaagaacggagaggtttactctccatcttctaagacttggacttctttgccaaatgctaaagttaaccctatgttgactgctgataagcaaggtttgtatagatctgataatcacgcttggttgtttggttggaagaaaggttctgttttccaagctggtccttctactgctatgaactggtactatacttctggttctggagatgttaagtctgctggtaaaagacaatctaatagaggtgttgctccagatgctatgtgtggtaacgctgttatgtacgatgctgttaagggtaaaattttgactttcggtggttctcctgattatcaagattctgatgctactactaacgctcatatcatcactttgggtgaaccaggtacttctcctaatactgtttttgcttctaacggtttgtactttgctagaactttccacacttctgttgttttgccagatggttctactttcattactggtggtcaaagaagaggtattccttttgaggattctactccagttttcactcctgaaatctacgttccagagcaagatactttctacaagcaaaaccctaattctatcgttagagtttaccattctatttctttgttgttgccagatggtagagttttcaacggtggtggtggtttgtgtggagattgtactactaaccacttcgatgctcaaattttcactccaaactacttgtacaactctaacggtaacttggctactagacctaagatcactagaacttctactcaatctgttaaggttggtggtagaatcactatttctactgattcttctatcactaaggcttctttgatcagatatggtactgctactcacactgttaatactgatcaaagaagaatcccattgactttgactaacaatggtggtaactcttactcttttcaagttccatctgattctggtgttgctttgcctggttattggatgttgttcgttatgaactctgctggtgttccttctgttgcttctactattagagttactcaagcggccgcactcgagcaccaccaccaccaccactga

SEQ ID NO: 2 - protein, 673 aa, Fusarium graminearum
MGSSHHHHHHSSGLVPRGSHMGTASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAALEHHHHHH

SEQ ID NO: 3 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatctcacgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccatgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgttcggcggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatggagtggcggagtgtttgagaagaatggcgaagtctatagcccctcatccaaaacgtggacgtcacttccgaacgcgaaggtgaatcccatgttaaccgccgacaaacagggcctgtaccgcagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactaccaggacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgtgtatcactccatcagccttcttctcccggacggacgtgtctttaatggcggaggaggactgtgcggggactgcacgaccaatcattttgacgcgcagatctttaccccgaattatctgtataattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 4 - protein, 665 aa, synthetic construct
MGSSHHHHHHSSGLVPRGSHMGTASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 5 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgttcgaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 6 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 7 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgttcgaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtactttagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 8 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYFSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 9 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgttcgaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcattttgtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 10 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFCGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 11 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgttcgaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggaggttgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 12 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGGCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 13 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggttgaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 14 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAVEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 15 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgccggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 16 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAPEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 17 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgcaggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 18 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAQEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 19 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgaatgaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 20 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDANEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 21 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgtctgaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 22 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDASEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 23 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgttcgaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 24 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAFEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 25 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgttccctggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 26 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAFPGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 27 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgattgaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 28 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAIEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 29 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgacggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 30 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDATEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 31 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgttggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 32 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDALEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 33 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggaggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 34 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAEEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 35 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgtatgaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 36 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAYEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 37 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 38 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 39 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgtgtgaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 40 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDACEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 41 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggatgaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 42 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDADEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 43 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgggtgaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 44 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAGEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 45 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgcatgaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 46 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAHEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 47 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgaaagaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 48 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAKEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 49 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgatggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 50 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAMEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 51 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgcgtgaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 52 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAREGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 53 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgccacctggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 54 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAPPGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 55 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgccggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggagggtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 56 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAPEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGGCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 57 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgccgccaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggagggtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 58 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAPPGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGGCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 59 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggttccaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 60 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAVPGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 61 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggttgaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggagggtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 62 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAVEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGGCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 63 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggttccaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggagggtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 64 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAVPGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGGCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 65 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgttcccaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggcaatgacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggagggtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 66 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAFPGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGGCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 67 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 68 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 69 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtactttagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 70 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYFSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 71 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcattttgtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 72 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFCGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 73 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgggtgaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 74 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAGEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 75 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcgggtgaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtactttagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 76 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAGEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYFSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 77 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccagcgccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 78 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPSAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 79 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcacccccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 80 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPITPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 81 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaccggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 82 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGTGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 83 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtcttcagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 84 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVFSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 85 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgcgtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 86 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARAYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 87 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccaccgccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 88 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPTAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 89 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgaccatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 90 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLTMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 91 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgtggcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 92 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVWPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 93 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagcctgtgcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 94 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAACAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 95 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgacggtaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 96 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDGTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 97 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcgattattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 98 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAIIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 99 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacagccagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 100 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADSQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 101 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccagattcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 102 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPDSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 103 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccacagtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 104 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPQSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 105 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaatttcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 106 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPISGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 107 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaaaatcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 108 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPKSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 109 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtatgtgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 110 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVCAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 111 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccctggacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 112 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTLDKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 113 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgatctcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 114 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVISAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 115 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtggatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 116 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLWMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 117 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgaaaggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 118 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLKGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 119 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgcgtggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 120 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLRGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 121 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatggaatgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 122 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMECPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 123 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgtattgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 124 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMYCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 125 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaagcatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 126 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDKHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 127 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgaccgtcatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 128 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDRHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 129 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgactggcatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 130 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDWHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 131 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatcccccgtgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 132 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPREDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 133 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactactgggacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 134 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYWDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 135 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcgaaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 136 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAKGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 137 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcacgtagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 138 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSRSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 139 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggtatccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 140 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWYPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 141 - DNA, 2001 bp, synthetic construct
  source 1..2001: mol_type = other DNA
atgggcagcagccatcatcatcatcatcacagcagcggcctggtgccgcgcggcagccatatgggtacccattctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 142 - protein, 665 aa, synthetic construct
MGSSHHHHHHSSGLVPRGSHMGTHSAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 143 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctcgtggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 144 - protein, 665 aa, synthetic construct
MGSSHHHHHHSSGLVPRGSHMGTASAPRGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 145 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatgggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 146 - protein, 665 aa, synthetic construct
MGSSHHHHHHSSGLVPRGSHMGTASAPMGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 147 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccaggatgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 148 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQDVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 149 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggagcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 150 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWSPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 151 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgccgtatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 152 - protein, 665 aa, synthetic construct
MGSSHHHHHHSSGLVPRGSHMGTASAPIGSAIPRNNWAVTCDSAQSGNECNKAIDGNKDTFWHTPYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 153 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgcgtgaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 154 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVREANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 155 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccggtgcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 156 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAGAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 157 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcagcggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 158 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVSGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 159 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcaccccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 160 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGTPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 161 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtagcgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 162 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRSDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 163 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcgcacccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 164 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGAPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 165 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtcgtgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 166 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRRDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 167 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctggaatgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcatcaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 168 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWNAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 169 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggccgtcccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 170 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGRPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 171 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcaaacccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 172 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGKPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 173 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaacgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 174 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSERTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 175 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcgataactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 176 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGDNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 177 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagccgtgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 178 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSRDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 179 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgatccgaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 180 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDPNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 181 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagcaatgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 182 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSNDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 183 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcactggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgaccccggccgcagcttgataa

SEQ ID NO: 184 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTPAAA

SEQ ID NO: 185 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagccgttataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 186 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASRYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 187 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccccgcgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 188 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSPRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 189 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccgaatcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 190 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQAESYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 191 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgaccgatgccgcagcttgataa

SEQ ID NO: 192 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTDAAA

SEQ ID NO: 193 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagcaaatataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 194 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASKYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 195 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgttgccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 196 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPVAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 197 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcgtttggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 198 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAFGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 199 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactcagacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 200 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITQTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 201 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggctggactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 202 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGWTLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 203 - DNA, 2001 bp, synthetic construct
  source 1..2001: mol_type = other DNA
atgggcagcagccatcatcatcatcatcacagcagcggcctggtgccgcgcggcagccatatgggtaccgcctctcgtcctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 204 - protein, 665 aa, synthetic construct
MGSSHHHHHHSSGLVPRGSHMGTASRPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 205 - DNA, 2001 bp, synthetic construct
  source 1..2001: mol_type = other DNA
atgggcagcagccatcatcatcatcatcacagcagcggcctggtgccgcgcggcagccatatgggtaccgcctctgccccttttggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 206 - protein, 665 aa, synthetic construct
MGSSHHHHHHSSGLVPRGSHMGTASAPFGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 207 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcgtatggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 208 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAYGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 209 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactgttacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 210 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITVTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 211 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctcccagcgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 212 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSQRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 213 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcacatggacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 214 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHMDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 215 - DNA, 2001 bp, synthetic construct
  source 1..2001: mol_type = other DNA
atgggcagcagccatcatcatcatcatcacagcagcggcctggtgccgcgcggcagccatatgggtaccaattctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 216 - protein, 665 aa, synthetic construct
MGSSHHHHHHSSGLVPRGSHMGTNSAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 217 - DNA, 2001 bp, synthetic construct
  source 1..2001: mol_type = other DNA
atgggcagcagccatcatcatcatcatcacagcagcggcctggtgccgcgcggcagccatatgggtaccgcctctcatcctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 218 - protein, 665 aa, synthetic construct
MGSSHHHHHHSSGLVPRGSHMGTASHPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 219 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgtatgggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 220 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRMGTATHTVNTDQRRIPLTLTNNGGNSYSFQVPSDSGVALPGYWMLFVMNSAGVPSVASTIRVTQAAA

SEQ ID NO: 221 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacaatcagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 222 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANNQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 223 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcggatagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 224 - protein, 665 aa, synthetic construct
MGSSHHHHHHSSGLVPRGSHMGTASAPIGSAIPRNNWAVTCDSADSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 225 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcggaaagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 226 - protein, 665 aa, synthetic construct
MGSSHHHHHHSSGLVPRGSHMGTASAPIGSAIPRNNWAVTCDSAESGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 227 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgaatagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 228 - protein, 665 aa, synthetic construct
MGSSHHHHHHSSGLVPRGSHMGTASAPIGSAIPRNNWAVTCDSANSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 229 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacaaatgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 230 - protein, 665 aa, synthetic construct
MGSSHHHHHHSSGLVPRGSHMGTASAPIGSAIPRNNWAVTCDSAQSGNKCNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 231 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcagatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 232 - protein, 665 aa, synthetic construct
MGSSHHHHHHSSGLVPRGSHMGTASAPIGSAIPRNNWAVTCDSAQSGNECNKAIDGNKDTFWHTFYGADGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 233 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcagaaggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 234 - protein, 665 aa, synthetic construct
MGSSHHHHHHSSGLVPRGSHMGTASAPIGSAIPRNNWAVTCDSAQSGNECNKAIDGNKDTFWHTFYGAEGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 235 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcatttggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 236 - protein, 665 aa, synthetic construct
MGSSHHHHHHSSGLVPRGSHMGTASAPIGSAIPRNNWAVTCDSAQSGNECNKAIDGNKDTFWHTFYGAFGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 237 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcacatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 238 - protein, 665 aa, synthetic construct
MGSSHHHHHHSSGLVPRGSHMGTASAPIGSAIPRNNWAVTCDSAQSGNECNKAIDGNKDTFWHTFYGAHGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 239 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcacttggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 240 - protein, 665 aa, synthetic construct
MGSSHHHHHHSSGLVPRGSHMGTASAPIGSAIPRNNWAVTCDSAQSGNECNKAIDGNKDTFWHTFYGALGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 241 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaatgggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 242 - protein, 665 aa, synthetic construct
MGSSHHHHHHSSGLVPRGSHMGTASAPIGSAIPRNNWAVTCDSAQSGNECNKAIDGNKDTFWHTFYGAMGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 243 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaacaggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 244 - protein, 665 aa, synthetic construct
MGSSHHHHHHSSGLVPRGSHMGTASAPIGSAIPRNNWAVTCDSAQSGNECNKAIDGNKDTFWHTFYGATGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 245 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcatggggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 246 - protein, 665 aa, synthetic construct
MGSSHHHHHHSSGLVPRGSHMGTASAPIGSAIPRNNWAVTCDSAQSGNECNKAIDGNKDTFWHTFYGAWGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 247 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcatatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 248 - protein, 665 aa, synthetic construct
MGSSHHHHHHSSGLVPRGSHMGTASAPIGSAIPRNNWAVTCDSAQSGNECNKAIDGNKDTFWHTFYGAYGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 249 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggaggtatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 250 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGGIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 251 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggaaatatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 252 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGNIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 253 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctatgaagcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 254 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYEAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 255 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctatcatgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 256 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYHAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 257 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctatattgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 258 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYIAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 259 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataaagcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 260 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYKAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 261 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggatccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 262 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVDPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 263 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccaccgccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtggatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 264 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPTAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLWMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 265 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgtggcccatgttaaccgccgacaaacagggcctgaccatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 266 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVWPMLTADKQGLTMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 267 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcgattattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaccggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 268 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAIIEPTSGRVLMWSSYRNDAAEGSPGGITLTSSWDPSTGIVSDRTVTVTKHDMFCPGISMDGTGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 269 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccagcgccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgtggcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 270 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPSAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVWPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 271 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgaaagaggcgaacggccagccatggacctcaatcgccgaaatcaatgtattccaagccagctcctataccgcgccgcagccaggacttgggcgttggggcccgaccatcgacctgcctatcgtacccagcgccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 272 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVKEANGQPWTSIAEINVFQASSYTAPQPGLGRWGPTIDLPIVPSAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 273 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctcgtggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgatctcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 274 - protein, 665 aa, synthetic construct
MGSSHHHHHHSSGLVPRGSHMGTASAPRGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVISAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 275 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgcgtgaggcgaacggccagccatggacctcaatcgccgaaatcaatgtattccaagccagctcctataccgcgccgcagccaggacttgggcgttggggcccgaccatcgacctgcctatcgtacccaccgccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 276 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVREANGQPWTSIAEINVFQASSYTAPQPGLGRWGPTIDLPIVPTAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 277 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctcgtggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgcgtgaggcgaacggccagccatggacctcaatcgccgaaatcaatgtattccaagccagctcctataccgcgccgcagccaggacttgggcgttggggcccgaccatcgacctgcctatcgtacccgcagccgcgattattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgtggcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 278 - protein, 665 aa, synthetic construct
MGSSHHHHHHSSGLVPRGSHMGTASAPRGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVREANGQPWTSIAEINVFQASSYTAPQPGLGRWGPTIDLPIVPAAAIIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVWPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 279 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccaccgccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaccggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgtggcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 280 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPTAAAIEPTSGRVLMWSSYRNDAAEGSPGGITLTSSWDPSTGIVSDRTVTVTKHDMFCPGISMDGTGQIVVTGGWDAKKTSLYDSSSDSWIPGPDMQVARGYQSSATMSDGRVFTIGGSFSGGVFEKNGEVYSPSSKTWTSLPNAKVWPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 281 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctcgtggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccaccgccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaccggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgatctcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 282 - protein, 665 aa, synthetic construct
MGSSHHHHHHSSGLVPRGSHMGTASAPRGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPTAAAIEPTSGRVLMWSSYRNDAAEGSPGGITLTSSWDPSTGIVSDRTVTVTKHDMFCPGISMDGTGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVISAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 283 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctcgtggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaccggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgtggcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 284 - protein, 665 aa, synthetic construct
MGSSHHHHHHSSGLVPRGSHMGTASAPRGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGTGQIVVTGGWDAKKTSLYDSSSDSWIPGPDMQVARGYQSSATMSDGRVFTIGGSFSGGVFEKNGEVYSPSSKTWTSLPNAKVWPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 285 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctcgtggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccagcgccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgatctcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 286 - protein, 665 aa, synthetic construct
MGSSHHHHHHSSGLVPRGSHMGTASAPRGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPSAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVISAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 287 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccaccgccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgtggcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 288 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPTAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVWPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 289 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctcgtggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccaccgccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgtggcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgatctcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 290 - protein, 665 aa, synthetic construct
MGSSHHHHHHSSGLVPRGSHMGTASAPRGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPTAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVWPMLTADKQGLYMSDNHAWLFGWKKGSVFQAGPSTAMNWYYTSGSGDVISAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 291 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctcgtggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccaccgccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaccggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgaatcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccgggggcgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 292 - protein, 665 aa, synthetic construct
MGSSHHHHHHSSGLVPRGSHMGTASAPRGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPTAAAIEPTSGRVLMWSSYRNDAAEGSPGGITLTSSWDPSTGIVSDRTVTVTKHDMFCPGISMDGTGQIVVTGGWDAKKTSLYDSSSDSWIPGPDMQVARGYQSSATMSDGRVFTIGGSFSGGVFEKNGEVYSPSSKTWTSLPNAKVNPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGASPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 293 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctcgtggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccagcgccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaccggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgtggcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 294 - protein, 665 aa, synthetic construct
MGSSHHHHHHSSGLVPRGSHMGTASAPRGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPSAAAIEPTSGRVLMWSSYRNDAAEGSPGGITLTSSWDPSTGIVSDRTVTVTKHDMFCPGISMDGTGQIVVTGGWDAKKTSLYDSSSDSWIPGPDMQVARGYQSSATMSDGRVFTIGGSFSGGVFEKNGEVYSPSSKTWTSLPNAKVWPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 295 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctcgtggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgacagaggcgaacggccagccatggacctcaatcgccgaaatcaatgtattccaagccagctcctataccgcgccgcagccaggacttgggcgttggggcccgaccatcgacctgcctatcgtacccaccgccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaccggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgtggcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgatctcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 296 - protein, 665 aa, synthetic construct
MGSSHHHHHHSSGLVPRGSHMGTASAPRGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPTAAAIEPTSGRVLMWSSYRNDAAEGSPGGITLTSSWDPSTGIVSDRTVTVTKHDMFCPGISMDGTGQIVVTGGWDAKKTSLYDSSSDSWIPGPDMQVARGYQSSATMSDGRVFTIGGSFSGGVFEKNGEVYSPSSKTWTSLPNAKVWPMLTADKQGLYMSDNHAWLFGWKKGSVFQAGPSTAMNWYYTSGSGDVISAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

SEQ ID NO: 297 - DNA, 2001 bp, synthetic construct
  source 1..2001: mol_type = other DNA
atgggcagcagccatcatcatcatcatcacagcagcggcctggtgccgcgcggcagccatatgggtaccgcctctgcccctatcggctcagccatcccgcgcaataactgggccgtaacgtgcgacagcgcgcagagcggcaacgagtgcaataaagccatcgacggcaataaagacaccttctggcacacgttttatggcgcaaatggcgacccgaagccaccgcatacctataccattgacatgaaaaccacccagaatgtgaacgggctgtccgtgctgccgcgccaggacggcaatcagaacggatggatcggccgtcacgaggtatatttaagctccgacggcaccaactggggatccccggtcgcgtcgggcagctggttcgccgacagcaccaccaagtattccaattttgaaacccgtcctgcacgctacgtgcgcttagtggccgtgcgtgaggcgaacggccagccatggacctcaatcgccgaaatcaatgtattccaagccagctcctataccgcgccgcagccaggacttgggcgttggggcccgaccatcgacctgcctatcgtacccgcagccgcggcgattgaaccaacgtcaggccgcgtactgatgtggagcagctaccgtaacgacgcggcggaaggcagcccgggcggcatcactctgacgtcgagttgggatccatccacgggcatcgtctccgaccgcaccgtgaccgtcacgaaacacgacatgttttgcccggggatcagcatggacggcaacggccagatcgtcgtcacgggcggctgggacgccaagaagacgagcctctatgacagtagcagcgacagttggatcccggggccggacatgcaggtggcacgcgggtaccagtccagcgcgaccatgtcagacggccgtgtctttacaatcggcggctcatttagtggcggagtgtttgagaagaatggcgaagtctatagcccctcatccaaaacgtggacgtcacttccgaacgcgaaggtgtggcccatgttaaccgccgacaaacagggcctgtacatgagcgacaaccatgcgtggctgttcggctggaagaaggggagcgtctttcaggcgggcccgtcgaccgcaatgaattggtattacacgtcaggaagcggggacgtgaaatcagccggcaagcgccagtcgaaccgcggcgtggcgccggacgcaatgtgcggcaatgccgtgatgtatgacgcggtcaaaggcaagatcctgacatttggcgggagtccggactacaccgacagtgacgcgacaaccaatgcccacattattacccttggggagccggggacgagcccgaacacggtcttcgcgtccaatggcctctatttcgcgcgtacctttcatacgtcggtggtcctgccggacggatcaacctttatcaccggcggacagcgccgtggcatccccttcgaagactcgacccccgtctttacaccggagatttatgtgccggaacaggacacgttttataaacagaatccgaactcgattgtacgcgcgtatcactccatcagccttcttctcccggacggacgtgtctttaatggcggaggaggactgtgcggggactgcacgaccaatcattttgacgcgcagatctttaccccgaattatctgtatgattcaaatgggaatctcgcgacccgtccgaaaattacccgtacatcaacccagtccgtcaaagtgggcggccgcattacgatcagtaccgacagcagcattaccaaagcgtcactgattcgttacggcaccgcaacccatacagtcaacacggaccagcgccgtattccgttaaccctgaccaataacggtggcaattcgtatagcttccaggtaccgtcggactcgggcgtggcgctgccaggatactggatgctgtttgtgatgaatagcgcgggcgtcccgagcgtagcgagcacgatccgtgtgacccaggccgcagcttgataa

SEQ ID NO: 298 - protein, 665 aa, synthetic construct
MGSSHHHHHHSSGLVPRGSHMGTASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAVREANGQPWTSIAEINVFQASSYTAPQPGLGRWGPTIDLPIVPAAAAIEPTSGRVLMWSSYRNDAAEGSPGGITLTSSWDPSTGIVSDRTVTVTKHDMFCPGISMDGNGQIVVTGGWDAKKTSLYDSSSDSWIPGPDMQVARGYQSSATMSDGRVFTIGGSFSGGVFEKNGEVYSPSSKTWTSLPNAKVWPMLTADKQGLYMSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQAAA

(19) United States
(12) Patent Application Publication
(10) Pub. No.: US 2026/0055379 A1
(43) Pub. Date: Feb. 26, 2026

(54) An Improved Galactose Oxidase Enzyme Panel for Oxidation of Secondary Alcohols

(71) Applicant: AGENCY FOR SCIENCE, TECHNOLOGY AND RESEARCH, Singapore (SG)

(72) Inventors: Yee Hwee Lim, Singapore (SG); Wei Peng Dillon Tay, Singapore (SG); Jhoann Margarette Tristeza Miyajima, Singapore (SG); Sebastian Maurer-Stroh, Singapore (SG); Wan Lin Yeo, Singapore (SG); Ee Lui Ang, Singapore (SG)

(21) Appl. No.: 19/099,603

(22) PCT Filed: Aug. 1, 2023
(86) PCT No.: PCT/SG2023/050534

§ 371 Date: Jan. 29, 2025

(30) Foreign Application Priority Data
Aug. 11, 2022 (SG) 10202250709V

(51) Int. Cl. C12N 9/04; C12P 7/26

(52) U.S. Cl. CPC C12N 9/0006; C12P 7/26; C12Y 101/03009

(57) ABSTRACT

Disclosed are modified galactose oxidase variants with enhanced protein expression, solubility, thermal stability, and enzymatic activity, and reduced enantioselectivity compared to wild-type galactose oxidase or galactose oxidase variants known in the art, and methods of producing the same.

### CROSS-REFERENCE TO RELATED APPLICATIONS

This application claims the benefit of priority of Singapore provisional application No. 10202250709V, filed 11 Aug. 2022, the contents of it being hereby incorporated by reference in its entirety for all purposes.

### TECHNICAL FIELD

The present disclosure generally relates to a modified galactose oxidase capable of oxidizing a wide range of secondary alcohols to ketones.

### BACKGROUND

Alcohol oxidation is an important transformation in chemical synthesis due to the convenience of hydroxyl and carbonyl functionalities as synthetic intermediates. Present chemical alcohol oxidation methodologies typically involve elevated temperatures, stoichiometric oxidants, and toxic by-products.

In contrast, biocatalytic alcohol oxidation by enzymes such as galactose oxidase possess advantages such as high selectivity, mild reaction conditions, benign by-products, and sustainable operation in water instead of organic solvents. However, one inherent drawback of excellent biocatalytic selectivity is their limited substrate scope.

Galactose oxidase is a radical-copper enzyme. Since its discovery, various research groups have worked on expanding its substrate scope. The most well studied variant of galactose oxidase for oxidation of secondary alcohols is the M3-5 variant, which comprises eight mutations (S33P, M93V, G218E, W313F, R353M, Q429T, V517A, N558D) with respect to wild-type galactose oxidase from Fusarium sp. M3-5 encompasses a relatively wide substrate scope, including selected benzylic secondary alcohols. However, no enzyme variants so far have been able to accept bulky secondary alcohols.

There is therefore a need to develop modified galactose oxidase enzymes that can oxidize a wide range of secondary alcohols to ketones, to create a panel of biocatalysts for an important transformation in chemical synthesis.

### SUMMARY

In one aspect, the present disclosure refers to a modified galactose oxidase comprising a polypeptide sequence having at least 85%, 86%, 87%, 88%, 89%, 90%, 91%, 92%, 93%, 94%, 95%, 96%, 97%, 98%, or 99% sequence identity to SEQ ID NO: 6, wherein SEQ ID NO: 6 is a polypeptide sequence of galactose oxidase variant M3-5, wherein the galactose oxidase variant M3-5 comprises mutations of S33P, M93V, G218E, W313F, R353M, Q429T, V517A, and N558D relative to the wildtype galactose oxidase from Fusarium graminearum (SEQ ID NO: 4), and wherein the polypeptide sequence of the modified galactose oxidase further comprises at least one mutation at one or more positions selected from the group consisting of 24, 26, 28, 45, 49, 65, 69, 104, 114, 117, 153, 157, 172, 173, 175, 193, 194, 195, 197, 198, 202, 214, 217, 218, 220, 224, 226, 239, 240, 250, 259, 265, 268, 269, 284, 294, 313, 314, 325, 341, 346, 348, 352, 353, 356, 389, 401, 429, 447, 485, 487, 537, 538, 546, 559, 576, 599, and 662, wherein the position of the mutation is numbered with reference to SEQ ID NO: 6.

In one aspect, the present disclosure refers to a polynucleotide sequence encoding the modified galactose oxidase disclosed herein.

In one aspect, the present disclosure refers to an expression vector comprising the polynucleotide sequence disclosed herein.

In one aspect, the present disclosure refers to a host cell comprising the polynucleotide sequence disclosed herein or the expression vector disclosed herein.

In one aspect, the present disclosure refers to a method of producing a modified galactose oxidase, comprising culturing the host cell disclosed herein under suitable culture conditions such that the modified galactose oxidase is produced.

In one aspect, the present disclosure refers to a method of producing a ketone from a secondary alcohol, comprising contacting the secondary alcohol with the modified galactose oxidase disclosed herein under suitable conditions for an oxidation reaction.

In one aspect, the present disclosure refers to a kit for use in the method disclosed herein, wherein the kit comprises the modified galactose oxidase disclosed herein.

### BRIEF DESCRIPTION OF DRAWINGS

The invention will be better understood with reference to the detailed description when considered in conjunction with the non-limiting examples and the accompanying drawings, in which:

FIG. 1 shows an expanded substrate scope of the identified galactose oxidase variants. Up to 64 substrates were tested were test in the panel.

FIG. 2 highlights the performance of top performing galactose oxidase variant against five (5) selected examples of bulky benzylic secondary alcohol substrates with reference to the M3-5 variant as benchmark.

FIG. 3 shows the mutation details on five (5) selected galactose oxidase variants (incorporating three or more synergistic mutations on top of the M3-5 variant) and their high performance liquid chromatography (HPLC) yields of ortho-substituted bulky benzylic secondary alcohol substrates compared to mutant GOh1052 as the benchmark.

FIG. 4 shows the enzyme profile of 13 top performing galactose oxidase variants in terms of activity (high performance liquid chromatography yield from secondary alcohol substrate 152, 1-phenyl-1-butanol), residual activity (described in FIG. 6), protein expression, and ratio of soluble to insoluble protein compared to GOh1052 variant (F217A, N268W from M3-5) as the benchmark.

FIG. 5 shows the measured colorimetric activities of 12 selected galactose oxidase variants as well as M3-5 benchmark against the 64 secondary alcohol substrate panel described in FIG. 1. Mutations are given in the top row, variant identifier numbers are given in the second row, and substrate identifier numbers are given in the first column. Colour coded heatmap comparisons have been done horizontally per substrate.

FIG. 6 shows the procedure for measuring residual activity of a galactose oxidase variant.

### DEFINITION OF TERMS

The following words and terms used herein shall have the meaning indicated:

As used in this application, the singular form “a,” “an,” and “the” include plural references unless the context clearly dictates otherwise. For example, the term “a primer” includes a plurality of primers, including mixtures and combinations thereof.

As used herein, the terms “increase” and “decrease” refer to the relative alteration of a chosen trait or characteristic in a subset of a population in comparison to the same trait or characteristic as present in the whole population. An increase thus indicates a change on a positive scale, whereas a decrease indicates a change on a negative scale. The term “change”, as used herein, also refers to the difference between a chosen trait or characteristic of an isolated population subset in comparison to the same trait or characteristic in the population as a whole. However, this term is without valuation of the difference seen.

As used herein, the term “about” in the context of concentration of a substance, size of a substance, length of time, or other stated values means+/−5% of the stated value, or +/−4% of the stated value, or +/−3% of the stated value, or +/−2% of the stated value, or +/−1% of the stated value, or +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the disclosures embodied therein herein disclosed may be resorted to by those skilled in the art, and that such modifications and variations are considered to be within the scope of this disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

Other embodiments are within the following claims and non-limiting examples. In addition, where features or aspects of the disclosure are described in terms of Markush groups, those skilled in the art will recognize that the disclosure is also thereby described in terms of any individual member or subgroup of members of the Markush group.

As used herein, the terms “modified galactose oxidase”, “galactose oxidase mutant” and “galactose oxidase variant” are used interchangeably, when referring to altered forms of a galactose oxidase resulting from genetic changes or modifications.

The secondary alcohol may include, but is not limited, a bulky secondary alcohol or an unactivated secondary alcohol. In one example, the bulky secondary alcohol comprises one or more bulky substituents. In one example, an unactivated secondary alcohol is an aliphatic secondary alcohol without a benzene ring in the alpha position adjacent to the alcohol group. The term “bulky” when used in relation to the secondary alcohol refers to an alcohol that is sterically bulky (or large or complex). In one example, the sterically bulky alcohol is an alkyl alcohol (carbon-based, C—OH). Examples of sterically bulky alcohols are 1-phenyl-1-butanol, diphenylmethanol, alpha-tetralol, and the like (such as those disclosed herein), as well as derivatives thereof. Similarly, the term “bulky” when used in relation to “substituents” refers to a substituent that is sterically bulky (or large or complex). In one example, the bulky secondary alcohol is an alcohol comprising one or more bulky substituents. In one example, the bulky substituent is selected from the group consisting of cycloalkyl, aryl, heteroaryl, alkyl, alkylene, alkynyl, haloalkyl, carboxyl, and carbonyl, wherein the cycloalkyl, aryl, heteroaryl, alkyl, alkylene, alkynyl, haloalkyl, carboxyl, and carbonyl is optionally substituted with one or more substituents selected from the group consisting of alkyl, halogen, alkyl halide, alkylene, alkynyl, alkoxy, cyano, oxo, nitro, amino, thiol, carboxyl, ester and hydroxyl. In one example, the secondary alcohol comprises the formula R¹R²CHOH, wherein R¹ and R² are independently selected from the group consisting of cycloalkyl, aryl, heteroaryl, alkyl, alkylene, alkynyl, haloalkyl, carboxyl, and carbonyl, wherein the cycloalkyl, aryl, heteroaryl, alkyl, alkylene, alkynyl, haloalkyl, carboxyl, and carbonyl is optionally substituted with one or more substituents selected from the group consisting of alkyl, halogen, alkyl halide, alkylene, alkynyl, alkoxy, cyano, oxo, nitro, amino, thiol, carboxyl, ester and hydroxyl.

The present disclosure includes within its scope all isomeric forms of the compounds disclosed herein, including all diastereomeric isomers, racemates and enantiomers. Thus, formulae (I) and (II) should be understood to include, for example, E, Z, cis, trans, (R), (S), (L), (D), (+), and/or (−) forms of the compounds, as appropriate in each case.

The term “optionally substituted” as used herein means the group to which this term refers may be unsubstituted, or may be substituted with one or more groups independently selected from alkyl, alkenyl, alkynyl, thioalkyl, cycloalkyl, cycloalkenyl, heterocycloalkyl, halo, carboxyl, haloalkyl, haloalkynyl, hydroxyl, alkoxy, thioalkoxy, alkenyloxy, haloalkoxy, haloalkenyloxy, nitro, amino, nitroalkyl, nitroalkenyl, nitroalkynyl, nitroheterocyclyl, alkylamino, dialkylamino, alkenylamine, alkynylamino, acyl, alkenoyl, alkynoyl, acylamino, diacylamino, acyloxy, alkylsulfonyloxy, heterocycloxy, heterocycloamino, haloheterocycloalkyl, alkylsulfenyl, alkylcarbonyloxy, alkylthio, acylthio, phosphorus-containing groups such as phosphono and phosphinyl, aryl, heteroaryl, alkylaryl, alkylheteroaryl, cyano, cyanate, isocyanate, —C(O)NH(alkyl), and —C(O)N(alkyl)₂.

The term “cycloalkyl” as used herein refers to cyclic saturated aliphatic groups and includes within its meaning monovalent (“cycloalkyl”), and divalent (“cycloalkylene”), saturated, monocyclic, bicyclic, polycyclic or fused polycyclic hydrocarbon radicals having from 3 to 10 carbon atoms, e.g., 3, 4, 5, 6, 7, 8, 9, or 10 carbon atoms. Examples of cycloalkyl groups include but are not limited to cyclopropyl, 2-methylcyclopropyl, cyclobutyl, cyclopentyl, 2-methylcyclopentyl, 3-methylcyclopentyl, cyclohexyl, and the like. In one example, the term “fused polycyclic hydrocarbon radical” refers to a fused polycyclic hydrocarbon group, ring, or system, wherein the carbon atoms within the fused polycyclic hydrocarbon group, ring or system are covalently bonded.

The term “aromatic group”, or variants such as “aryl” or “arylene” as used herein refers to monovalent (“aryl”) and divalent (“arylene”) single, polynuclear, conjugated and fused residues of aromatic hydrocarbons having from 6 to 10 carbon atoms. Examples of such groups include phenyl, biphenyl, naphthyl, phenanthrenyl, and the like.

The term “heteroaromatic group” and variants such as “heteroaryl” or “heteroarylene” as used herein, includes within its meaning monovalent (“heteroaryl”) and divalent (“heteroarylene”), single, polynuclear, conjugated and fused aromatic radicals having 6 to 20 atoms wherein 1 to 6 atoms are heteroatoms selected from O, N, NH and S. Examples of such groups include pyridyl, 2,2′-bipyridyl, phenanthrolinyl, quinolinyl, thiophenyl, and the like. In one example, the term “fused aromatic radical” refers to a fused aromatic group, ring, or system, wherein the atoms within the fused aromatic group, ring, or system are covalently bonded.

As used herein, the term “alkyl” includes within its meaning monovalent (“alkyl”) and divalent (“alkylene”) straight chain or branched chain saturated aliphatic groups having from 1 to 10 carbon atoms, e.g., 1, 2, 3, 4, 5, 6, 7, 8, 9, or 10 carbon atoms. For example, the term alkyl includes, but is not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, and the like.

The term “alkenyl” includes within its meaning monovalent (“alkenyl”) and divalent (“alkenylene”) straight or branched chain unsaturated aliphatic hydrocarbon groups having from 2 to 10 carbon atoms, e.g., 2, 3, 4, 5, 6, 7, 8, 9, or 10 carbon atoms and having at least one double bond, of either E, Z, cis or trans stereochemistry where applicable, anywhere in the alkyl chain. Examples of alkenyl groups include but are not limited to ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 1-nonenyl, 1-decenyl, and the like.

The term “alkynyl” as used herein includes within its meaning monovalent (“alkynyl”) and divalent (“alkynylene”) straight or branched chain unsaturated aliphatic hydrocarbon groups having from 2 to 10 carbon atoms and having at least one triple bond anywhere in the carbon chain. Examples of alkynyl groups include but are not limited to ethynyl, 1-propynyl, 1-butynyl, 2-butynyl, 1-methyl-2-butynyl, 3-methyl-1-butynyl, 1-pentynyl, 1-hexynyl, methylpentynyl, 1-heptynyl, 2-heptynyl, 1-octynyl, 2-octynyl, 1-nonyl, 1-decynyl, and the like.

The term “haloalkyl” refers to a straight-or branched-chain alkenyl group having from 2-12 carbon atoms in the chain and where one or more hydrogens is substituted with a halogen. Illustrative haloalkyl groups include trifluoromethyl, 2-bromopropyl, 3-chlorohexyl, 1-iodo-isobutyl, and the like.

The term “carboxyl” as used herein is intended to mean the radical-C(O) OH.

The term “carbonyl” as used herein refers to a functional group comprising a carbon atom with a double bond to an oxygen atom, which can be written as C(═O) or as C(O).

The term “halogen” or variants such as “halide” or “halo” as used herein refers to fluorine, chlorine, bromine and iodine.

The term “alkyl halide” as used herein includes within its meaning straight or branched chain alkyl groups having from 2-12 carbon atoms and in which one or more of its hydrogen atoms are substituted with the corresponding number of halogen atoms. An alkyl halide with Cₙ carbon atoms may have at least one up to 2ₙ+1 halogen atoms, and the halogen atoms may be the same or different. Examples of alkyl halide groups include but are not limited to CF₃, C₂F₅, CHF₂, CHCl₂, CHBr₂, C₂Cl₅ and the like.

The term “alkoxy” as used herein refers to straight chain or branched alkyloxy groups. Examples include methoxy, ethoxy, n-propoxy, isopropoxy, tert-butoxy, and the like.

The term “cyano”, used interchangeably with “nitrile”, refers to a group comprising a carbon atom with a triple bond to a nitrogen atom, which can be written as —C≡N or —CN.

The term “oxo” as used herein is intended to mean the radical ═O.

The term “amino” as used herein refers to groups of the form —NRₐR_{b} wherein Rₐ and R_{b} are individually selected from the group including but not limited to hydrogen, optionally substituted alkyl, optionally substituted alkenyl, optionally substituted alkynyl, and optionally substituted aryl groups.

The term “nitro” as used herein refers to a functional group comprising a nitrogen atom joined to two oxygen atoms, which can be written as —O—N═O or —NO₂.

The term “thiol” as used herein refers to a group comprising the formula —SH.

The term “ester” as used herein refers to a group represented by the general formula R_{c}COOR_{d}. R_{c} and/or R_{d} may include, but is not limited to alkyl, aryl, haloalkyl.

The term “hydroxyl” as used herein refers to the group —OH.

In the phrase “even numbered sequences”, the term “even number” refers to any number that is a multiple of 2. In one example, even numbered sequences of SEQ ID NOS: 1-298 refer to SEQ ID NOS: 2, 4, 6, 8, 10, 12, 14, 16, 18, 20, 22, and so on until SEQ ID NO: 298. In another example, even numbered sequences of SEQ ID NOS: 77-298 refer to SEQ ID NOS: 78, 80, 82, 84, 86, 88, 90, 92, 94, 96, 98, 100, 102, and so on until SEQ ID NO: 298.

In the phrase “odd numbered sequences”, the term “odd number” refers to any number that is not a multiple of 2. In one example, odd numbered sequences of SEQ ID NOS: 1-298 refer to SEQ ID NOS: 1, 3, 5, 7, 9, 11, 13, 15, 17, 19, 21, and so on until SEQ ID NO: 297. In another example, odd numbered sequences of SEQ ID NOS: 77-298 refer to SEQ ID NOS: 77, 79, 81, 83, 85, 87, 89, 91, 93, 95, 97, 99, 101 and so on until SEQ ID NO: 297.

### DETAILED DESCRIPTION

The present disclosure is directed to a panel of modified galactose oxidase enzymes based on copper-dependent galactose oxidase from Fusarium graminearum. The present disclosure discloses 118 unique mutations (either singly or in combination) from the backbone M3-5 variant that convey improved enhancement in activity and/or stability, and/or solubility, and/or protein expression, and/or selectivity compared to M3-5 variant. The present disclosure describes galactose oxidase variants with enhanced protein expression, solubility, thermal stability and enzymatic activity, and reduced enantioselectivity, compared to galactose oxidase variants known in the art, and methods of producing the same. Galactose oxidases catalyse the oxidation of secondary alcohol substrates to ketones in accordance with the chemical equation shown below:

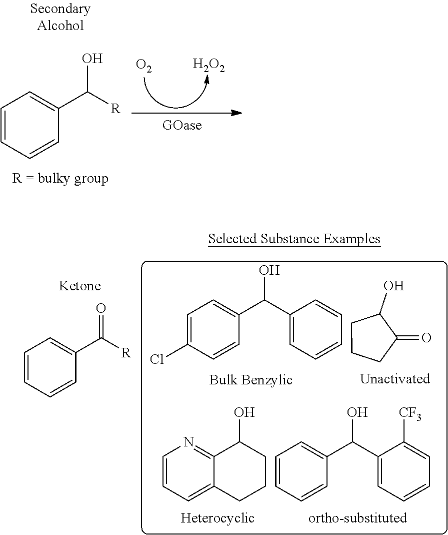

The inventors therefore screened and identified galactose oxidase variants that are capable of accepting a wide range of bulky secondary alcohols (e.g., biphenyl, C₈ alkyl sidechain, ortho-substitution) with good tolerance of halogen (e.g., Cl, Br), alkene, alkyne, and nitro functional groups. The galactose oxidase variants disclosed herein are also capable of accepting unactivated secondary alcohols as substrates.

In one aspect, the present disclosure refers to a modified galactose oxidase comprising a polypeptide sequence having at least 85%, 86%, 87%, 88%, 89%, 90%, 91%, 92%, 93%, 94%, 95%, 96%, 97%, 98%, or 99% sequence identity to SEQ ID NO: 6, wherein SEQ ID NO: 6 is a polypeptide sequence of galactose oxidase variant M3-5, wherein the galactose oxidase variant M3-5 comprises mutations of S33P, M93V, G218E, W313F, R353M, Q429T, V517A, and N558D relative to the wildtype galactose oxidase from Fusarium graminearum (SEQ ID NO: 4), and wherein the polypeptide sequence of the modified galactose oxidase further comprises at least one mutation at one or more positions selected from the group consisting of 24, 26, 28, 45, 49, 65, 69, 104, 114, 117, 153, 157, 172, 173, 175, 193, 194, 195, 197, 198, 202, 214, 217, 218, 220, 224, 226, 239, 240, 250, 259, 265, 268, 269, 284, 294, 313, 314, 325, 341, 346, 348, 352, 353, 356, 389, 401, 429, 447, 485, 487, 537, 538, 546, 559, 576, 599, and 662, wherein the position of the mutation is numbered with reference to SEQ ID NO: 6.

In one example, the present disclosure refers to the modified galactose oxidase disclosed herein, wherein the mutation is selected from the group consisting of A24H, A24N, A26H, A26R, I28F, I28M, I28R, Q45D, Q45E, Q45N, E49K, F65P, N69D, N69E, N69F, N69H, N69L, N69M, N69T, N69W, N69Y, W104G, W104N, S114N, S114R, T117D, T153K, T153R, G157N, S172E, S173K, S173R, T175E, T175H, T175I, T175K, V193T, P194C, A195S, A195T, A195V, A197C, A197G, A198I, T202D, T202I, T202K, T202Q, N214R, N214S, F217A, F217C, F217D, F217E, F217G, F217H, F217I, F217K, F217L, F217M, F217N, F217P, F217Q, F217R, F217S, F217T, F217V, F217Y, E218F, E218K, E218P, E218Y, S220T, I224W, L226Q, L226V, D239E, D239P, D239Q, R240G, F250E, F250Y, N259T, T265S, N268W, D269N, I284S, I284Y, G294A, F313R, S314C, Y325F, N341W, A346L, K348S, Y352T, Y352W, M353F, N356K, N356R, N356W, K389I, A401D, T429W, T447A, I485A, I485K, I485R, F487R, L537G, C538K, C538R, F546M, S559P, S576D, Y599M, Q662D, and Q662P.

In one specific example, the present disclosure refers to the modified galactose oxidase disclosed herein, wherein the mutation is selected from the group consisting of I28R, A24H, A195S, A195T, A195V, A198I, T202I, F217A, E218F, E218K, L226Q, D239E, N259T, N268W, S314C, Y352W, Y599M, and combinations thereof.

In one example, the present disclosure refers to the modified galactose oxidase disclosed herein, wherein the mutation is selected from the group consisting of:
M353F;
S314C;
L537G;
F217V;
F217P;
F217Q;
F217N;
F217S;
N268W;
E218P;
F217I;
F217T;
F217L;
F217E;
F217Y;
F217A;
F217C;
F217D;
F217G;
F217H;
F217K;
F217M;
F217R;
a combination of F217P and E218P;
a combination of F217P and L537G;
a combination of F217P, E218P and L537G;
a combination of F217V and E218P;
a combination of F217V and L537G;
a combination of F217V, E218P and L537G;
a combination of E218P and L537G;
a combination of F217A and N268W;
a combination of F217A, N268W and M353F;
a combination of F217A, N268W and S314C;
a combination of F217G and N268W;
a combination of F217G, N268W and M353F;
a combination of F217A, N268W and A195S;
a combination of F217A, N268W and V193T;
a combination of F217A, N268W and N259T;
a combination of F217A, N268W and Y325F;
a combination of F217A, N268W and G294A;
a combination of F217A, N268W and A195T;
a combination of F217A, N268W and Y352T;
a combination of F217A, N268W and N341W;
a combination of F217A, N268W and A197C;
a combination of F217A, N268W and R240G;
a combination of F217A, N268W and A198I;
a combination of F217A, N268W and K348S;
a combination of F217A, N268W and T202D;
a combination of F217A, N268W and T202Q;
a combination of F217A, N268W and T202I;
a combination of F217A, N268W and T202K;
a combination of F217A, N268W and P194C;
a combination of F217A, N268W and A346L;
a combination of F217A, N268W and K389I;
a combination of F217A, N268W and Y352W;
a combination of F217A, N268W and C538K;
a combination of F217A, N268W and C538R;
a combination of F217A, N268W and F250E;
a combination of F217A, N268W and F250Y;
a combination of F217A, N268W and N356K;
a combination of F217A, N268W and N356R;
a combination of F217A, N268W and N356W;
a combination of F217A, N268W and F487R;
a combination of F217A, N268W and T429W;
a combination of F217A, N268W and E218K;
a combination of F217A, N268W and F313R;
a combination of F217A, N268W and I284Y;
a combination of F217A, N268W and A24H;
a combination of F217A, N268W and I28R;
a combination of F217A, N268W and I28M;
a combination of F217A, N268W and S576D;
a combination of F217A, N268W and I284S;
a combination of F217A, N268W and F65P;
a combination of F217A, N268W and T153R;
a combination of F217A, N268W and A197G;
a combination of F217A, N268W and T265S;
a combination of F217A, N268W and S220T;
a combination of F217A, N268W and N214S;
a combination of F217A, N268W and I485A;
a combination of F217A, N268W and N214R;
a combination of F217A, N268W and D269N;
a combination of F217A, N268W and I485R;
a combination of F217A, N268W and I485K;
a combination of F217A, N268W and D239E;
a combination of F217A, N268W and Ti17D;
a combination of F217A, N268W and S114R;
a combination of F217A, N268W and S559P;
a combination of F217A, N268W and S114N;
a combination of F217A, N268W and Q662P;
a combination of F217A, N268W and S173R;
a combination of F217A, N268W and D239P;
a combination of F217A, N268W and S172E;
a combination of F217A, N268W and Q662D;
a combination of F217A, N268W and S173K;
a combination of F217A, N268W and A195V;
a combination of F217A, N268W and E218F;
a combination of F217A, N268W and L226Q;
a combination of F217A, N268W and I224W;
a combination of F217A, N268W and A26R;
a combination of F217A, N268W and I28F;
a combination of F217A, N268W and E218Y;
a combination of F217A, N268W and L226V;
a combination of F217A, N268W and D239Q;
a combination of F217A, N268W and F546M;
a combination of F217A, N268W and A24N;
a combination of F217A, N268W and A26H;
a combination of F217A, N268W and Y599M;
a combination of F217A, N268W and G157N;
a combination of F217A, N268W and Q45D;
a combination of F217A, N268W and Q45E;
a combination of F217A, N268W and Q45N;
a combination of F217A, N268W and E49K;
a combination of F217A, N268W and N69D;
a combination of F217A, N268W and N69E;
a combination of F217A, N268W and N69F;
a combination of F217A, N268W and N69H;
a combination of F217A, N268W and N69L;
a combination of F217A, N268W and N69M;
a combination of F217A, N268W and N69T;
a combination of F217A, N268W and N69W;
a combination of F217A, N268W and N69Y;
a combination of F217A, N268W and W104G;
a combination of F217A, N268W and W104N;
a combination of F217A, N268W and T175E;
a combination of F217A, N268W and T175H;
a combination of F217A, N268W and T175I;
a combination of F217A, N268W and T175K;
a combination of F217A, N268W and A401D;
a combination of F217A, N268W, A195T and Y352W;
a combination of F217A, N268W, N341W and Y352T;
a combination of F217A, N268W, A198I and N259T;
a combination of F217A, N268W, A195S and N341W;
a combination of F217A, N268W, T153K and A195S;
a combination of F217A, N268W, I28R and K389I;
a combination of F217A, N268W, T153R and A195T;
a combination of F217A, N268W, I28R, T153R, A198I and N341W;
a combination of F217A, N268W, A195T, N259T and N341W;
a combination of F217A, N268W, I28R, A195T, N259T and K389I;
a combination of F217A, N268W, I28R, N259T and N341W;
a combination of F217A, N268W, I28R, A195S and K389I;
a combination of F217A, N268W, A195T and N341W;
a combination of F217A, N268W, I28R, A195T, N341W and K389I;
a combination of F217A, N268W, I28R, A195T, N259T and T447A;
a combination of F217A, N268W, I28R, A195S, N259T and N341W;
a combination of F217A, N268W, I28R, A195T, N259T, N341W and K389I; and
a combination of F217A, N268W, T153R and N341W.

In one example, there is provided a galactose oxidase variant GOh1006, which has a M353F mutation in addition to those in the M3-5 variant. In one example, there is provided a galactose oxidase variant GOh1008, which has a S314C mutation in addition to those in the M3-5 variant. In one example, there is provided a galactose oxidase variant GOh1013, which has a L537G mutation in addition to those in the M3-5 variant. In one example, there is provided a galactose oxidase variant GOh1015, which has a F217V mutation in addition to those in the M3-5 variant. In one example, there is provided a galactose oxidase variant GOh1016, which has a F217P mutation in addition to those in the M3-5 variant. In one example, there is provided a galactose oxidase variant GOh1017, which has a F217Q mutation in addition to those in the M3-5 variant. In one example, there is provided a galactose oxidase variant GOh1018, which has a F217N mutation in addition to those in the M3-5 variant. In one example, there is provided a galactose oxidase variant GOh1020, which has a F217S mutation in addition to those in the M3-5 variant. In one example, there is provided a galactose oxidase variant GOh1021, which has a N268W mutation in addition to those in the M3-5 variant. In one example, there is provided a galactose oxidase variant GOh1025, which has a E218P mutation in addition to those in the M3-5 variant. In one example, there is provided a galactose oxidase variant GOhM030, which has a F217I mutation in addition to those in the M3-5 variant. In one example, there is provided a galactose oxidase variant GOh1031, which has a F217T mutation in addition to those in the M3-5 variant. In one example, there is provided a galactose oxidase variant GOh1032, which has a F217L mutation in addition to those in the M3-5 variant. In one example, there is provided a galactose oxidase variant GOh1034, which has a F217E mutation in addition to those in the M3-5 variant. In one example, there is provided a galactose oxidase variant GOh1035, which has a F217Y mutation in addition to those in the M3-5 variant. In one example, there is provided a galactose oxidase variant GOh1036, which has a F217A mutation in addition to those in the M3-5 variant. In one example, there is provided a galactose oxidase variant GOh1037, which has a F217C mutation in addition to those in the M3-5 variant. In one example, there is provided a galactose oxidase variant GOh1038, which has a F217D mutation in addition to those in the M3-5 variant. In one example, there is provided a galactose oxidase variant GOh1039, which has a F217G mutation in addition to those in the M3-5 variant. In one example, there is provided a galactose oxidase variant GOh1040, which has a F217H mutation in addition to those in the M3-5 variant. In one example, there is provided a galactose oxidase variant GOh1041, which has a F217K mutation in addition to those in the M3-5 variant. In one example, there is provided a galactose oxidase variant GOh1042, which has a F217M mutation in addition to those in the M3-5 variant. In one example, there is provided a galactose oxidase variant GOh1043, which has a F217R mutation in addition to those in the M3-5 variant. In one example, there is provided a galactose oxidase variant GOh1045, which has F217P and E218P mutations in addition to those in the M3-5 variant. In one example, a modified galactose oxidase having the F217P and E218P mutations corresponds to galactose oxidase variant GOh1045. In one example, there is provided a galactose oxidase variant GOh1046, which has FF217P and L537G mutations in addition to those in the M3-5 variant. In one example, there is provided a galactose oxidase variant GOh1047, which has F217P, E218P, and L537G mutations in addition to those in the M3-5 variant. In one example, there is provided a galactose oxidase variant GOh1048, which has F217V and E218P mutations in addition to those in the M3-5 variant. In one example, there is provided a galactose oxidase variant GOh1049, which has F217V and L537G mutations in addition to those in the M3-5 variant. In one example, there is provided a galactose oxidase variant GOh1050, which has F217V, E218P, and L537G mutations in addition to those in the M3-5 variant. In one example, there is provided a galactose oxidase variant GOh1051, which has E218P and L537G mutations in addition to those in the M3-5 variant. In one example, there is provided a galactose oxidase variant GOh1052, which has F217A and N268W mutations in addition to those in the M3-5 variant. In one example, there is provided a galactose oxidase variant GOh1053, which has F217A, N268W, and M353F mutations in addition to those in the M3-5 variant. In one example, there is provided a galactose oxidase variant GOh1054, which has F217A, N268W, and S314C mutations in addition to those in the M3-5 variant. In one example, there is provided a galactose oxidase variant GOh1055, which has F217G and N268W mutations in addition to those in the M3-5 variant. In one example, there is provided a galactose oxidase variant GOh1056, which has F217G, N268W, and M353 mutations in addition to those in the M3-5 variant. In one example, there is provided a galactose oxidase variant GOh2001, which has F217A, N268W, and A195S mutations in addition to those in the M3-5 variant. In one example, there is provided a galactose oxidase variant GOh2002, which has F217A, N268W, and V193T mutations in addition to those in the M3-5 variant. In one example, there is provided a galactose oxidase variant GOh2008, which has F217A, N268W, and N259T mutations in addition to those in the M3-5 variant. In one example, there is provided a galactose oxidase variant GOh2010, which has F217A, N268W, and Y325F mutations in addition to those in the M3-5 variant. In one example, there is provided a galactose oxidase variant GOh2011, which has F217A, N268W, and G294A mutations in addition to those in the M3-5 variant. In one example, there is provided a galactose oxidase variant GOh2012, which has F217A, N268W, and A195T mutations in addition to those in the M3-5 variant. In one example, there is provided a galactose oxidase variant GOh2013, which has F217A, N268W, and Y352T mutations in addition to those in the M3-5 variant. In one example, there is provided a galactose oxidase variant GOh2014, which has F217A, N268W, and N341W mutations in addition to those in the M3-5 variant. In one example, there is provided a galactose oxidase variant GOh2015, which has F217A, N268W, and A197C mutations in addition to those in the M3-5 variant. In one example, there is provided a galactose oxidase variant GOh2016, which has F217A, N268W, and R240G mutations in addition to those in the M3-5 variant. In one example, there is provided a galactose oxidase variant GOh2017, which has F217A, N268W, and A198I mutations in addition to those in the M3-5 variant. In one example, there is provided a galactose oxidase variant GOh2018, which has F217A, N268W, and K348S mutations in addition to those in the M3-5 variant. In one example, there is provided a galactose oxidase variant GOh2019, which has F217A, N268W, and T202D mutations in addition to those in the M3-5 variant. In one example, there is provided a galactose oxidase variant GOh2020, which has F217A, N268W, and T202Q mutations in addition to those in the M3-5 variant. In one example, there is provided a galactose oxidase variant GOh2021, which has F217A, N268W, and T202I mutations in addition to those in the M3-5 variant. In one example, there is provided a galactose oxidase variant GOh2022, which has F217A, N268W, and T202K mutations in addition to those in the M3-5 variant. In one example, there is provided a galactose oxidase variant GOh2023, which has F217A, N268W, and P194C mutations in addition to those in the M3-5 variant. In one example, there is provided a galactose oxidase variant GOh2025, which has F217A, N268W, and A346L mutations in addition to those in the M3-5 variant. In one example, there is provided a galactose oxidase variant GOh2026, which has F217A, N268W, and K389I mutations in addition to those in the M3-5 variant. In one example, there is provided a galactose oxidase variant GOh2027, which has F217A, N268W, and Y352W mutations in addition to those in the M3-5 variant. In one example, there is provided a galactose oxidase variant GOh2028, which has F217A, N268W, and C538K mutations in addition to those in the M3-5 variant. In one example, there is provided a galactose oxidase variant GOh2029, which has F217A, N268W, and C538R mutations in addition to those in the M3-5 variant. In one example, there is provided a galactose oxidase variant GOh2030, which has F217A, N268W, and F250E mutations in addition to those in the M3-5 variant. In one example, there is provided a galactose oxidase variant GOh2031, which has F217A, N268W, and F250Y mutations in addition to those in the M3-5 variant. In one example, there is provided a galactose oxidase variant GOh2032, which has F217A, N268W, and N356K mutations in addition to those in the M3-5 variant. In one example, there is provided a galactose oxidase variant GOh2033, which has F217A, N268W, and N356R mutations in addition to those in the M3-5 variant. In one example, there is provided a galactose oxidase variant GOh2034, which has F217A, N268W, and N356W mutations in addition to those in the M3-5 variant. In one example, there is provided a galactose oxidase variant GOh2037, which has F217A, N268W, and F487R mutations in addition to those in the M3-5 variant. In one example, there is provided a galactose oxidase variant GOh2038, which has F217A, N268W, and T429W mutations in addition to those in the M3-5 variant. In one example, there is provided a galactose oxidase variant GOh2039, which has F217A, N268W, and E218K mutations in addition to those in the M3-5 variant. In one example, there is provided a galactose oxidase variant GOh2041, which has F217A, N268W, and F313R mutations in addition to those in the M3-5 variant. In one example, there is provided a galactose oxidase variant GOh2043, which has F217A, N268W, and I284Y mutations in addition to those in the M3-5 variant. In one example, there is provided a galactose oxidase variant GOh2044, which has F217A, N268W, and A24H mutations in addition to those in the M3-5 variant. In one example, there is provided a galactose oxidase variant GOh2045, which has F217A, N268W, and I28R mutations in addition to those in the M3-5 variant. In one example, there is provided a galactose oxidase variant GOh2046, which has F217A, N268W, and I28M mutations in addition to those in the M3-5 variant. In one example, there is provided a galactose oxidase variant GOh2047, which has F217A, N268W, and S576D mutations in addition to those in the M3-5 variant. In one example, there is provided a galactose oxidase variant GOh2048, which has F217A, N268W, and I284S mutations in addition to those in the M3-5 variant. In one example, there is provided a galactose oxidase variant GOh2050, which has F217A, N268W, and F65P mutations in addition to those in the M3-5 variant. In one example, there is provided a galactose oxidase variant GOh2053, which has F217A, N268W, and T153R mutations in addition to those in the M3-5 variant. In one example, there is provided a galactose oxidase variant GOh2055, which has F217A, N268W, and A197G mutations in addition to those in the M3-5 variant. In one example, there is provided a galactose oxidase variant GOh2056, which has F217A, N268W, and T265S mutations in addition to those in the M3-5 variant. In one example, there is provided a galactose oxidase variant GOh2057, which has F217A, N268W, and S220T mutations in addition to those in the M3-5 variant. In one example, there is provided a galactose oxidase variant GOh2058, which has F217A, N268W, and N214S mutations in addition to those in the M3-5 variant. In one example, there is provided a galactose oxidase variant GOh2061, which has F217A, N268W, and I485A mutations in addition to those in the M3-5 variant. In one example, there is provided a galactose oxidase variant GOh2064, which has F217A, N268W, and N214R mutations in addition to those in the M3-5 variant. I In one example, there is provided a galactose oxidase variant GOh2066, which has F217A, N268W, and D269N mutations in addition to those in the M3-5 variant. In one example, there is provided a galactose oxidase variant GOh2067, which has F217A, N268W, and I485R mutations in addition to those in the M3-5 variant. In one example, there is provided a galactose oxidase variant GOh2068, which has F217A, N268W, and I485K mutations in addition to those in the M3-5 variant. In one example, there is provided a galactose oxidase variant GOh2070, which has F217A, N268W, and D239E mutations in addition to those in the M3-5 variant. In one example, there is provided a galactose oxidase variant GOh2071, which has F217A, N268W, and T117D mutations in addition to those in the M3-5 variant. In one example, there is provided a galactose oxidase variant GOh2072, which has F217A, N268W, and S114R mutations in addition to those in the M3-5 variant. In one example, there is provided a galactose oxidase variant GOh2073, which has F217A, N268W, and S559P mutations in addition to those in the M3-5 variant. In one example, there is provided a galactose oxidase variant GOh2074, which has F217A, N268W, and S114N mutations in addition to those in the M3-5 variant. In one example, there is provided a galactose oxidase variant GOh2075, which has F217A, N268W, and Q662P mutations in addition to those in the M3-5 variant. In one example, there is provided a galactose oxidase variant GOh2076, which has F217A, N268W, and S173R mutations in addition to those in the M3-5 variant. In one example, there is provided a galactose oxidase variant GOh2077, which has F217A, N268W, and D239P mutations in addition to those in the M3-5 variant. In one example, there is provided a galactose oxidase variant GOh2078, which has F217A, N268W, and S172E mutations in addition to those in the M3-5 variant. In one example, there is provided a galactose oxidase variant GOh2079, which has F217A, N268W, and Q662D mutations in addition to those in the M3-5 variant. In one example, there is provided a galactose oxidase variant GOh2080, which has F217A, N268W, and S173K mutations in addition to those in the M3-5 variant. In one example, there is provided a galactose oxidase variant GOh2081, which has F217A, N268W, and A195V mutations in addition to those in the M3-5 variant. In one example, there is provided a galactose oxidase variant GOh2082, which has F217A, N268W, and E218F mutations in addition to those in the M3-5 variant. In one example, there is provided a galactose oxidase variant GOh2083, which has F217A, N268W, and L226Q mutations in addition to those in the M3-5 variant. In one example, there is provided a galactose oxidase variant GOh2084, which has F217A, N268W, and I224W mutations in addition to those in the M3-5 variant. In one example, there is provided a galactose oxidase variant GOh2085, which has F217A, N268W, and I28F mutations in addition to those in the M3-5 variant. In one example, there is provided a galactose oxidase variant GOh2086, which has F217A, N268W, and I28F mutations in addition to those in the M3-5 variant. In one example, there is provided a galactose oxidase variant GOh2087, which has F217A, N268W, and E218Y mutations in addition to those in the M3-5 variant. In one example, there is provided a galactose oxidase variant GOh2088, which has F217A, N268W, and L226V mutations in addition to those in the M3-5 variant. In one example, there is provided a galactose oxidase variant GOh2089, which has F217A, N268W, and D239Q mutations in addition to those in the M3-5 variant. In one example, there is provided a galactose oxidase variant GOh2090, which has F217A, N268W, and F546M mutations in addition to those in the M3-5 variant. In one example, there is provided a galactose oxidase variant GOh2091, which has F217A, N268W, and A24N mutations in addition to those in the M3-5 variant. In one example, there is provided a galactose oxidase variant GOh2092, which has F217A, N268W, and A26H mutations in addition to those in the M3-5 variant. In one example, there is provided a galactose oxidase variant GOh2093, which has F217A, N268W, and Y599M mutations in addition to those in the M3-5 variant. In one example, there is provided a galactose oxidase variant GOh2094, which has F217A, N268W, and G157N mutations in addition to those in the M3-5 variant. In one example, there is provided a galactose oxidase variant GOh2097, which has F217A, N268W, and Q45D mutations in addition to those in the M3-5 variant. In one example, there is provided a galactose oxidase variant GOh2098, which has F217A, N268W, and Q45E mutations in addition to those in the M3-5 variant. In one example, there is provided a galactose oxidase variant GOh2099, which has F217A, N268W, and Q45N mutations in addition to those in the M3-5 variant. In one example, there is provided a galactose oxidase variant GOh2100, which has F217A, N268W, and E49K mutations in addition to those in the M3-5 variant. In one example, there is provided a galactose oxidase variant GOh2101, which has F217A, N268W, and N69D mutations in addition to those in the M3-5 variant. In one example, there is provided a galactose oxidase variant GOh2102, which has F217A, N268W, and N69E mutations in addition to those in the M3-5 variant. In one example, there is provided a galactose oxidase variant GOh2103, which has F217A, N268W, and N69F mutations in addition to those in the M3-5 variant. In one example, there is provided a galactose oxidase variant GOh2104, which has F217A, N268W, and N69H mutations in addition to those in the M3-5 variant. In one example, there is provided a galactose oxidase variant GOh2105, which has F217A, N268W, and N69L mutations in addition to those in the M3-5 variant. In one example, there is provided a galactose oxidase variant GOh2106, which has F217A, N268W, and N69M mutations in addition to those in the M3-5 variant. In one example, there is provided a galactose oxidase variant GOh2107, which has F217A, N268W, and N69T mutations in addition to those in the M3-5 variant. In one example, there is provided a galactose oxidase variant GOh2108, which has F217A, N268W, and N69W mutations in addition to those in the M3-5 variant. In one example, there is provided a galactose oxidase variant GOh2109, which has F217A, N268W, and N69Y mutations in addition to those in the M3-5 variant. In one example, there is provided a galactose oxidase variant GOh2110, which has F217A, N268W, and W104G mutations in addition to those in the M3-5 variant. In one example, there is provided a galactose oxidase variant GOh2111, which has F217A, N268W, and W104N mutations in addition to those in the M3-5 variant. In one example, there is provided a galactose oxidase variant GOh2l14, which has F217A, N268W, and T175E mutations in addition to those in the M3-5 variant. In one example, there is provided a galactose oxidase variant GOh2115, which has F217A, N268W, and T175H mutations in addition to those in the M3-5 variant. In one example, there is provided a galactose oxidase variant GOh2116, which has F217A, N268W, and T175I mutations in addition to those in the M3-5 variant. In one example, there is provided a galactose oxidase variant GOh2117, which has F217A, N268W, and T175K mutations in addition to those in the M3-5 variant. In one example, there is provided a galactose oxidase variant GOh2l19, which has F217A, N268W, and A401D mutations in addition to those in the M3-5 variant. In one example, there is provided a galactose oxidase variant GOh3001, which has F217A, N268W, A195T, and Y352W mutations in addition to those in the M3-5 variant. In one example, there is provided a galactose oxidase variant GOh3002, which has F217A, N268W, N341W, and Y352T mutations in addition to those in the M3-5 variant. In one example, there is provided a galactose oxidase variant GOh3003, which has F217A, N268W, A198I, and N259T mutations in addition to those in the M3-5 variant. In one example, there is provided a galactose oxidase variant GOh3004, which has F217A, N268W, A195S, and N341W mutations in addition to those in the M3-5 variant. In one example, there is provided a galactose oxidase variant GOh3005, which has F217A, N268W, T153K, and A195S mutations in addition to those in the M3-5 variant. In one example, there is provided a galactose oxidase variant GOh3006, which has F217A, N268W, I28R, and K389I mutations in addition to those in the M3-5 variant. In one example, there is provided a galactose oxidase variant GOh3007, which has F217A, N268W, T153R, and A195T mutations in addition to those in the M3-5 variant. In one example, there is provided a galactose oxidase variant GOh3008, which has F217A, N268W, I28R, T153R, A198I, and N341W mutations in addition to those in the M3-5 variant. In one example, there is provided a galactose oxidase variant GOh3009, which has F217A, N268W, A195T, N259T, and N341W mutations in addition to those in the M3-5 variant. In one example, there is provided a galactose oxidase variant GOh3013, which has F217A, N268W, I28R, A195T, N259T, and K389I mutations in addition to those in the M3-5 variant. In one example, there is provided a galactose oxidase variant GOh3015, which has F217A, N268W, I28R, N259T, and N341W mutations in addition to those in the M3-5 variant. In one example, there is provided a galactose oxidase variant GOh3018, which has F217A, N268W, I28R, A195S, and K389I mutations in addition to those in the M3-5 variant. In one example, there is provided a galactose oxidase variant GOh3019, which has F217A, N268W, A195T, and N341W mutations in addition to those in the M3-5 variant. In one example, there is provided a galactose oxidase variant GOh3023, which has F217A, N268W, I28R, A195T, N341W mutations in addition to those in the M3-5 variant. In one example, there is provided a galactose oxidase variant GOh3026, which has F217A, N268W, I28R, A195T, N259T, and T447A mutations in addition to those in the M3-5 variant. In one example, there is provided a galactose oxidase variant GOh3032, which has F217A, N268W, I28R, A195S, N259T, and N341W mutations in addition to those in the M3-5 variant. In one example, there is provided a galactose oxidase variant GOh3038, which has F217A, N268W, I28R, A195T, N259T, N341W, and K389I mutations in addition to those in the M3-5 variant. In one example, there is provided a galactose oxidase variant GOh3049, which has F217A, N268W, T153R, and N341W mutations in addition to those in the M3-5 variant. In one example, a modified galactose oxidase having the F217A, N268W, T153R, and N341W mutations corresponds to galactose oxidase variant GOh3049.

In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence of at least one modified galactose oxidase variant set forth in the even numbered sequences of SEQ ID NOS: 7-298. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence of at least one modified galactose oxidase variant set forth in the following even numbered sequences: SEQ ID NOS: 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, or 298. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 8. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 10. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 12. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 14. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 16. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 18. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 20. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 40. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 60. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 80. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 100. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 150. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 200. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 250. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 298.

In one example, the present disclosure refers to the modified galactose oxidase disclosed herein, wherein the polypeptide sequence of the modified galactose oxidase comprises a polypeptide sequence set forth in the even numbered sequences of SEQ ID NOS: 7-298. In one example, the present disclosure refers to the modified galactose oxidase disclosed herein, wherein the polypeptide sequence of the modified galactose oxidase comprises a polypeptide sequence set forth in the following even numbered sequences: SEQ ID NOS: 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, or 298. In one example, the present disclosure refers to the modified galactose oxidase disclosed herein, wherein the polypeptide sequence of the modified galactose oxidase comprises a polypeptide sequence set forth in SEQ ID NO: 8. In one example, the present disclosure refers to the modified galactose oxidase disclosed herein, wherein the polypeptide sequence of the modified galactose oxidase comprises a polypeptide sequence set forth in SEQ ID NO: 10. In one example, the present disclosure refers to the modified galactose oxidase disclosed herein, wherein the polypeptide sequence of the modified galactose oxidase comprises a polypeptide sequence set forth in SEQ ID NO: 12. In one example, the present disclosure refers to the modified galactose oxidase disclosed herein, wherein the polypeptide sequence of the modified galactose oxidase comprises a polypeptide sequence set forth in SEQ ID NO: 14. In one example, the present disclosure refers to the modified galactose oxidase disclosed herein, wherein the polypeptide sequence of the modified galactose oxidase comprises a polypeptide sequence set forth in SEQ ID NO: 16. In one example, the present disclosure refers to the modified galactose oxidase disclosed herein, wherein the polypeptide sequence of the modified galactose oxidase comprises a polypeptide sequence set forth in SEQ ID NO: 18. In one example, the present disclosure refers to the modified galactose oxidase disclosed herein, wherein the polypeptide sequence of the modified galactose oxidase comprises a polypeptide sequence set forth in SEQ ID NO: 20. In one example, the present disclosure refers to the modified galactose oxidase disclosed herein, wherein the polypeptide sequence of the modified galactose oxidase comprises a polypeptide sequence set forth in SEQ ID NO: 40. In one example, the present disclosure refers to the modified galactose oxidase disclosed herein, wherein the polypeptide sequence of the modified galactose oxidase comprises a polypeptide sequence set forth in SEQ ID NO: 60. In one example, the present disclosure refers to the modified galactose oxidase disclosed herein, wherein the polypeptide sequence of the modified galactose oxidase comprises a polypeptide sequence set forth in SEQ ID NO: 80. In one example, the present disclosure refers to the modified galactose oxidase disclosed herein, wherein the polypeptide sequence of the modified galactose oxidase comprises a polypeptide sequence set forth in SEQ ID NO: 100. In one example, the present disclosure refers to the modified galactose oxidase disclosed herein, wherein the polypeptide sequence of the modified galactose oxidase comprises a polypeptide sequence set forth in SEQ ID NO: 150. In one example, the present disclosure refers to the modified galactose oxidase disclosed herein, wherein the polypeptide sequence of the modified galactose oxidase comprises a polypeptide sequence set forth in SEQ ID NO: 200. In one example, the present disclosure refers to the modified galactose oxidase disclosed herein, wherein the polypeptide sequence of the modified galactose oxidase comprises a polypeptide sequence set forth in SEQ ID NO: 250. In one example, the present disclosure refers to the modified galactose oxidase disclosed herein, wherein the polypeptide sequence of the modified galactose oxidase comprises a polypeptide sequence set forth in SEQ ID NO: 298.

In one example, the present disclosure refers to the modified galactose oxidase disclosed herein, wherein the mutation is selected from the group consisting of:
(a) S314C;
(b) N268W;
(c) F217A;
(d) a combination of F217A and N268W;
(e) a combination of F217A, N268W and A195S;
(f) a combination of F217A, N268W and N259T;
(g) a combination of F217A, N268W and A195T;
(h) a combination of F217A, N268W and A198I;
(i) a combination of F217A, N268W and T202I;
(j) a combination of F217A, N268W and Y352W;
(k) a combination of F217A, N268W and A24H;
(l) a combination of F217A, N268W and I28R;
(m) a combination of F217A, N268W and D239E;
(n) a combination of F217A, N268W and A24N;
(o) a combination of F217A, N268W and E218K;
(p) a combination of F217A, N268W and E218F;
(q) a combination of F217A, N268W and Y599M;
(r) a combination of F217A, N268W and L226Q;
(s) a combination of F217A, N268W and A195V;
(t) a combination of F217A, N268W, A195T and Y352W;
(u) a combination of F217A, N268W, I28R and K389I;
(v) a combination of F217A, N268W, I28R, A195T, N259T, and K389I; and
(w) a combination of F217A, N268W, I28R, A195T, N259T, N341W, and K389I.

In one example, the present disclosure refers to the modified galactose oxidase disclosed herein, wherein the modified galactose oxidase comprises the mutations of F217A and N268W.

In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to SEQ ID NO:68.

In one example, the present disclosure refers to the modified galactose oxidase disclosed herein, wherein the modified galactose oxidase further comprises at least one mutation selected from the group consisting of A24H, A24N, A26H, A26R, I28F, I28M, I28R, Q45D, Q45E, Q45N, E49K, F65P, N69D, N69E, N69F, N69H, N69L, N69M, N69T, N69W, N69Y, W104G, W104N, S114N, S114R, T117D, T153K, T153R, G157N, S172E, S173K, S173R, T175E, T175H, T175I, T175K, V193T, P194C, A195SX, A195T, A195V, A197C, A197G, A198I, T202D, T202I, T202K, T202Q, N214R, N214S, E218F, E218K, E218P, E218Y, S220T, I224W, L226Q, L226V, D239E, D239P, D239Q, R240G, F250E, F250Y, N259T, T265S, D269N, I284S, I284Y, G294A, F313R, S314C, Y325F, N341W, A346L, K348S, Y352T, Y352W, M353F, N356K, N356R, N356W, K389I, A401D, T429W, T447A, I485A, I485K, I485R, F487R, L537G, C538K, C538R, F546M, S559P, S576D, Y599M, Q662D, and Q662P, wherein the position of the mutation is numbered with reference to SEQ ID NO: 6.

In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence of at least one modified galactose oxidase variant set forth in SEQ ID NOS: 70, 72 or the even numbered sequences of SEQ ID NOS: 77-298. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 70. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 72. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence of at least one modified galactose oxidase variant set forth in the following even numbered sequences: SEQ ID NOS: 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, or 298. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 78. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 80. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 90. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 90. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 100. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 120. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 140. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 160. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 180. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 200. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 250. In one example, the present disclosure refers to the modified galactose oxidase disclosed herein, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence set forth in SEQ ID NO: 298.

In one example, the present disclosure refers to the modified galactose oxidase disclosed herein, wherein the polypeptide sequence of the modified galactose oxidase comprises a polypeptide sequence set forth in SEQ ID NOS: 70, 72 or the even numbered sequences of SEQ ID NOS: 77-298. In one example, the present disclosure refers to the modified galactose oxidase disclosed herein, wherein the polypeptide sequence of the modified galactose oxidase comprises a polypeptide sequence set forth in SEQ ID NO: 70. In one example, the present disclosure refers to the modified galactose oxidase disclosed herein, wherein the polypeptide sequence of the modified galactose oxidase comprises a polypeptide sequence set forth in SEQ ID NO: 72. In one example, the present disclosure refers to the modified galactose oxidase disclosed herein, wherein the polypeptide sequence of the modified galactose oxidase comprises a polypeptide sequence set forth in the following even numbered sequences: SEQ ID NOS: 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, or 298. In one example, the present disclosure refers to the modified galactose oxidase disclosed herein, wherein the polypeptide sequence of the modified galactose oxidase comprises a polypeptide sequence set forth in SEQ ID NO: 78. In one example, the present disclosure refers to the modified galactose oxidase disclosed herein, wherein the polypeptide sequence of the modified galactose oxidase comprises a polypeptide sequence set forth in SEQ ID NO: 80. In one example, the present disclosure refers to the modified galactose oxidase disclosed herein, wherein the polypeptide sequence of the modified galactose oxidase comprises a polypeptide sequence set forth in SEQ ID NO: 90. In one example, the present disclosure refers to the modified galactose oxidase disclosed herein, wherein the polypeptide sequence of the modified galactose oxidase comprises a polypeptide sequence set forth in SEQ ID NO: 100. In one example, the present disclosure refers to the modified galactose oxidase disclosed herein, wherein the polypeptide sequence of the modified galactose oxidase comprises a polypeptide sequence set forth in SEQ ID NO: 120. In one example, the present disclosure refers to the modified galactose oxidase disclosed herein, wherein the polypeptide sequence of the modified galactose oxidase comprises a polypeptide sequence set forth in SEQ ID NO: 140. In one example, the present disclosure refers to the modified galactose oxidase disclosed herein, wherein the polypeptide sequence of the modified galactose oxidase comprises a polypeptide sequence set forth in SEQ ID NO: 160. In one example, the present disclosure refers to the modified galactose oxidase disclosed herein, wherein the polypeptide sequence of the modified galactose oxidase comprises a polypeptide sequence set forth in SEQ ID NO: 180. In one example, the present disclosure refers to the modified galactose oxidase disclosed herein, wherein the polypeptide sequence of the modified galactose oxidase comprises a polypeptide sequence set forth in SEQ ID NO: 200. In one example, the present disclosure refers to the modified galactose oxidase disclosed herein, wherein the polypeptide sequence of the modified galactose oxidase comprises a polypeptide sequence set forth in SEQ ID NO: 250. In one example, the present disclosure refers to the modified galactose oxidase disclosed herein, wherein the polypeptide sequence of the modified galactose oxidase comprises a polypeptide sequence set forth in SEQ ID NO: 298.

In one example, the present disclosure refers to the modified galactose oxidase disclosed herein, wherein the modified galactose oxidase comprises one or more of the following properties:
(a) enhanced protein expression by at least 1.01-fold compared to the galactose oxidase variant M3-5 or by at least 1.01-fold compared to the modified galactose oxidase disclosed herein;
(b) enhanced solubility by at least 1.01-fold compared to the galactose oxidase variant M3-5 or by at least 1.01-fold compared to the modified galactose oxidase disclosed herein;
(c) enhanced thermal stability by at least 1.01-fold compared to the galactose oxidase variant M3-5 or by at least 1.01-fold compared to the modified galactose oxidase disclosed herein at a temperature of at least 50° C.;
(d) increased enzymatic activity by at least 1.01-fold compared to the galactose oxidase variant M3-5 or by at least 1.01-fold compared to the modified galactose oxidase disclosed herein, wherein the enzymatic activity comprises converting a secondary alcohol to a ketone, wherein optionally the secondary alcohol is a bulky secondary alcohol or unactivated secondary alcohol; and
(e) reduced enantioselectivity by at least 1.01-fold compared to the galactose oxidase variant M3-5 or by at least 1.01-fold compared to the modified galactose oxidase disclosed herein.

In one example, the present disclosure refers to the modified galactose oxidase disclosed herein, wherein the modified galactose oxidase has enhanced protein expression by at least 1.01-fold compared to the galactose oxidase variant M3-5 or by at least 1.01-fold compared to the modified galactose oxidase variant GOh1052 that has F217A and N268W mutations. In one example, the modified galactose oxidase has enhanced protein expression by about 1.01 to 1.73-fold compared to the galactose oxidase variant M3-5. In one example, the modified galactose oxidase has enhanced protein expression by at least about 1.06-fold compared to the galactose oxidase variant M3-5. In one example, the modified galactose oxidase has enhanced protein expression by at least about 1.1-fold compared to the galactose oxidase variant M3-5. In one example, the modified galactose oxidase has enhanced protein expression by at least about 1.2-fold compared to the galactose oxidase variant M3-5. In one example, the modified galactose oxidase has enhanced protein expression by at least about 1.3-fold compared to the galactose oxidase variant M3-5. In one example, the modified galactose oxidase has enhanced protein expression by at least about 1.4-fold compared to the galactose oxidase variant M3-5. In one example, the modified galactose oxidase has enhanced protein expression by at least about 1.5-fold compared to the galactose oxidase variant M3-5. In one example, the modified galactose oxidase has enhanced protein expression by at least about 1.6-fold compared to the galactose oxidase variant M3-5. In one example, the modified galactose oxidase has enhanced protein expression by at least about 1.7-fold compared to the galactose oxidase variant M3-5. In one example, the modified galactose oxidase has enhanced protein expression by about 1.01 to 10.09-fold compared to the galactose oxidase variant GOh1052. In one example, the modified galactose oxidase has enhanced protein expression by at least about 1.01-fold compared to the galactose oxidase variant GOh1052. In one example, the modified galactose oxidase has enhanced protein expression by at least about 2-fold compared to the galactose oxidase variant GOh1052. In one example, the modified galactose oxidase has enhanced protein expression by at least about 3-fold compared to the galactose oxidase variant GOh1052. In one example, the modified galactose oxidase has enhanced protein expression by at least about 4-fold compared to the galactose oxidase variant GOh1052. In one example, the modified galactose oxidase has enhanced protein expression by at least about 5-fold compared to the galactose oxidase variant GOh1052. In one example, the modified galactose oxidase has enhanced protein expression by at least about 6-fold compared to the galactose oxidase variant GOh1052. In one example, the modified galactose oxidase has enhanced protein expression by at least about 7-fold compared to the galactose oxidase variant GOh1052. In one example, the modified galactose oxidase has enhanced protein expression by at least about 8-fold compared to the galactose oxidase variant GOh1052. In one example, the modified galactose oxidase has enhanced protein expression by at least about 9-fold compared to the galactose oxidase variant GOh1052. In one example, the modified galactose oxidase has enhanced protein expression by at least about 10-fold compared to the galactose oxidase variant GOh1052. In one example, the modified galactose oxidase has enhanced protein expression by at least about 10-fold compared to the galactose oxidase variant GOh1052. In one specific example, the modified galactose oxidase has enhanced protein expression by at least 1.51-fold compared to the galactose oxidase variant M3-5. In one specific example, the modified galactose oxidase has enhanced protein expression by at least 2.58-fold compared to the galactose oxidase variant GOh1052. In one specific example, the modified galactose oxidase has enhanced protein expression by at least 4.29-fold compared to the galactose oxidase variant GOh1052.

In one example, the present disclosure refers to the modified galactose oxidase disclosed herein, wherein the modified galactose oxidase has enhanced solubility by at least 1.01-fold compared to the galactose oxidase variant M3-5 or by at least 1.01-fold compared to the modified galactose oxidase variant GOh1052 that has the F217A and N268W mutations. In one example, the modified galactose oxidase has enhanced solubility by about 1.01 to 1.67-fold compared to the galactose oxidase variant M3-5. In one example, the modified galactose oxidase has enhanced solubility by at least about 1.1-fold compared to the galactose oxidase variant M3-5. In one example, the modified galactose oxidase has enhanced solubility by at least about 1.2-fold compared to the galactose oxidase variant M3-5. In one example, the modified galactose oxidase has enhanced solubility by at least about 1.3-fold compared to the galactose oxidase variant M3-5. In one example, the modified galactose oxidase has enhanced solubility by at least about 1.4-fold compared to the galactose oxidase variant M3-5. In one example, the modified galactose oxidase has enhanced solubility by at least about 1.5-fold compared to the galactose oxidase variant M3-5. In one example, the modified galactose oxidase has enhanced solubility by at least about 1.6-fold compared to the galactose oxidase variant M3-5. In one specific example, the modified galactose oxidase has enhanced solubility by at least about 1.13-fold compared to the galactose oxidase variant M3-5. In one example, the modified galactose oxidase has enhanced solubility by about 1.01 to 4.57-fold compared to the galactose oxidase variant GOh1052. In one example, the modified galactose oxidase has enhanced solubility by at least about 1.01-fold compared to the galactose oxidase variant GOh1052. In one example, the modified galactose oxidase has enhanced solubility by at least about 1.02-fold compared to the galactose oxidase variant GOh1052. In one example, the modified galactose oxidase has enhanced solubility by at least about 2-fold compared to the galactose oxidase variant GOh1052. In one example, the modified galactose oxidase has enhanced solubility by at least about 3-fold compared to the galactose oxidase variant GOh1052. In one example, the modified galactose oxidase has enhanced solubility by at least about 4-fold compared to the galactose oxidase variant GOh1052. In one specific example, the modified galactose oxidase has enhanced solubility by at least about 1.01-fold compared to the galactose oxidase variant GOh1052. In one specific example, the modified galactose oxidase has enhanced solubility by at least about 2.82-fold compared to the galactose oxidase variant GOh1052. In one specific example, the modified galactose oxidase has enhanced solubility by at least about 4.57-fold compared to the galactose oxidase variant GOh1052. In one example, solubility of the galactose oxidase variant is determined using the ratio of protein expression of soluble proteins over insoluble proteins. The higher the value, the more soluble it is. Solubility is then compared against either M3-5 (SEQ ID NO.: 6) or GOh1052 (SEQ ID NO.: 68), where the fold improvement is determined.

In one example, the present disclosure refers to the modified galactose oxidase disclosed herein, wherein the modified galactose oxidase has enhanced thermal stability by at least 1.01-fold compared to the modified galactose oxidase variant GOh1052 that has F217A and N268W mutations at a temperature of at least 50° C. or by at least 1.52-fold compared to the modified galactose oxidase variant M3-5 at a temperature of at least 55° C. In one example, the modified galactose oxidase has enhanced thermal stability by about 1.52 to 1.61-fold compared to the galactose oxidase variant M3-5. In one example, the modified galactose oxidase has enhanced thermal stability by at least about 1.6-fold compared to the galactose oxidase variant M3-5. In one specific example, the modified galactose oxidase has enhanced thermal stability by at least about 1.52-fold compared to the galactose oxidase variant M3-5. In one example, the modified galactose oxidase has enhanced thermal stability by about 1.01 to 1.61-fold compared to the galactose oxidase variant GOh1052. In one example, the modified galactose oxidase has enhanced thermal stability by at least about 1.01-fold compared to the galactose oxidase variant GOh1052. In one example, the modified galactose oxidase has enhanced thermal stability by at least about 1.1-fold compared to the galactose oxidase variant GOh1052. In one example, the modified galactose oxidase has enhanced thermal stability by at least about 1.2-fold compared to the galactose oxidase variant GOh1052. In one example, the modified galactose oxidase has enhanced thermal stability by at least about 1.3-fold compared to the galactose oxidase variant GOh1052. In one example, the modified galactose oxidase has enhanced thermal stability by at least about 1.4-fold compared to the galactose oxidase variant GOh1052. In one example, the modified galactose oxidase has enhanced thermal stability by at least about 1.5-fold compared to the galactose oxidase variant GOh1052. In one example, the modified galactose oxidase has enhanced thermal stability by at least about 1.6-fold compared to the galactose oxidase variant GOh1052. In one specific example, the modified galactose oxidase has enhanced thermal stability by at least about 1.50-fold compared to the galactose oxidase variant GOh1052. In one specific example, the modified galactose oxidase has enhanced thermal stability by at least about 1.61-fold compared to the galactose oxidase variant GOh1052. In one example, thermal stability of the galactose oxidase variants is tested using a two-phase approach. In one example, higher temperatures of 50° C. and 55° C. were tested in the two-phase approach by: (1) heating the enzyme at temperature for 30 minutes and then followed by (2) a contacting step of the heated enzyme and substrate combined at 25° C. for a specified reaction time, typically for 24 hours. In one example, 50° C. was the temperature at which the enzymes were incubated at for 30 minutes before being used in the standard 25° C. activity assay, which was done to assess thermal stability of the enzyme by comparison of the thermally challenged activity against enzyme activity that was not thermally challenged. In one example, 55° C. was the temperature at which the enzymes were incubated at for 30 minutes before being used in the standard 25° C. activity assay, which was done to assess thermal stability of the enzyme by comparison of the thermally challenged activity against enzyme activity that was not thermally challenged. These are distinct from the 40° C. activity assay, which is an activity assay conducted at higher temperatures to assess enzyme performance at higher temperatures. Although it can also be taken as a measure of thermal stability, the measure used to assess thermal stability was the former (50° C. for 30 minutes thermal challenge assay).

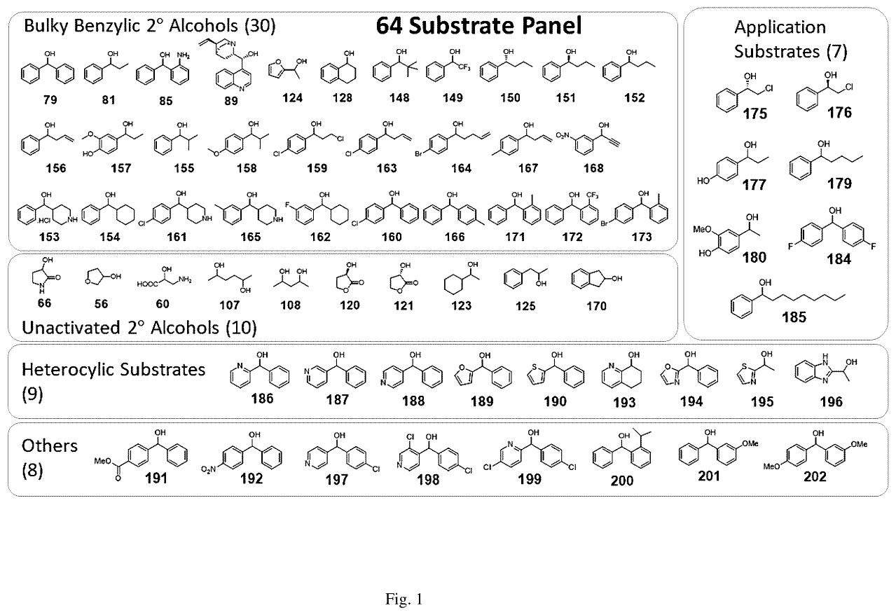

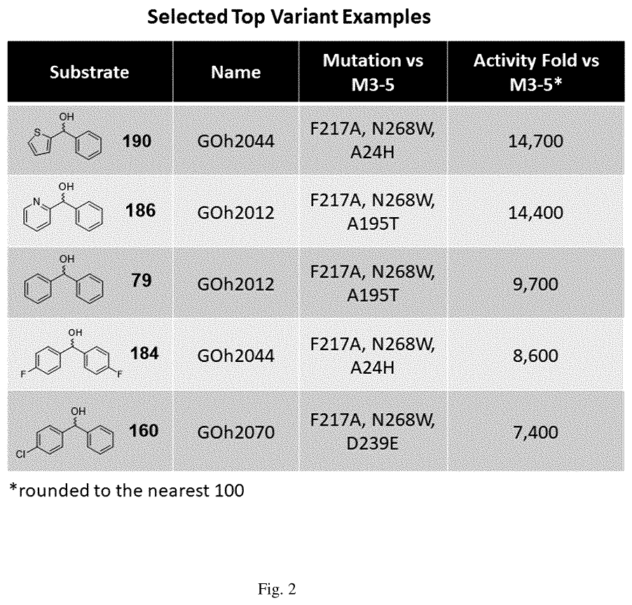

In one example, the present disclosure refers to the modified galactose oxidase disclosed herein, wherein the modified galactose oxidase has increased enzymatic activity by at least 1.01-fold compared to the galactose oxidase variant M3-5 or by at least 1.01-fold compared to the modified galactose oxidase variant GOh1052 that has F217A and N268W mutations, wherein the enzymatic activity comprises converting a secondary alcohol to a ketone, wherein optionally the secondary alcohol is a bulky secondary alcohol or unactivated secondary alcohol. In one example, the modified galactose oxidase has increased enzymatic activity by at least 1.1-fold compared to the galactose oxidase variant M3-5. In one example, the modified galactose oxidase has increased enzymatic activity by at least 2-fold compared to the galactose oxidase variant M3-5. In one example, the modified galactose oxidase has increased enzymatic activity by at least 3-fold compared to the galactose oxidase variant M3-5. In one example, the modified galactose oxidase has increased enzymatic activity by at least 4-fold compared to the galactose oxidase variant M3-5. In one example, the modified galactose oxidase has increased enzymatic activity by at least 5-fold compared to the galactose oxidase variant M3-5. In one example, the modified galactose oxidase has increased enzymatic activity by at least 6-fold compared to the galactose oxidase variant M3-5. In one example, the modified galactose oxidase has increased enzymatic activity by at least 7-fold compared to the galactose oxidase variant M3-5. In one example, the modified galactose oxidase has increased enzymatic activity by at least 8-fold compared to the galactose oxidase variant M3-5. In one example, the modified galactose oxidase has increased enzymatic activity by at least 9-fold compared to the galactose oxidase variant M3-5. In one specific example, the modified galactose oxidase has increased enzymatic activity by 1.85-fold compared to the galactose oxidase variant M3-5. In one specific example, the modified galactose oxidase has increased enzymatic activity by 2.18-fold compared to the galactose oxidase variant M3-5. In one specific example, the modified galactose oxidase has increased enzymatic activity by at least 1.5-fold better than M3-5 for Substrate 128 (alpha-tetralol). In one specific example, the modified galactose oxidase has increased enzymatic activity by at least 1.5-fold better than M3-5 for Substrate 128 (alpha-tetralol). In one specific example, the modified galactose oxidase has increased enzymatic activity by at least 1.04-fold better than GOh1052 for substrate 152 (1-phenyl-1-butanol). In one specific example, the modified galactose oxidase has increased enzymatic activity by at least 1.04-fold better than GOh1052 for substrate 152 (1-phenyl-1-butanol). In one specific example, the modified galactose oxidase has increased enzymatic activity by at least 1.01-fold better than M3-5 for 1 or more substrates in the 64-substrate panel in FIG. 1. In one specific example, the modified galactose oxidase has increased enzymatic activity by at least 1.1-fold better than M3-5 for 1 or more substrates in the 64-substrate panel in FIG. 1. In one example, the present disclosure refers to the modified galactose oxidase disclosed herein, wherein the modified galactose oxidase comprises increased enzymatic activity by at least 10-fold compared to the galactose oxidase variant M3-5 or by at least 1.2-fold compared to the modified galactose oxidase disclosed herein, wherein the enzymatic activity comprises converting a secondary alcohol to a ketone, wherein optionally the secondary alcohol is a bulky secondary alcohol or unactivated secondary alcohol.

In one example, the present disclosure refers to the modified galactose oxidase disclosed herein, reduced enantioselectivity by at least 1.04-fold compared to the galactose oxidase variant M3-5 or by at least 1.15-fold compared to the galactose oxidase variant GOh1052. In one example, the modified galactose oxidase disclosed herein, reduced enantioselectivity by about 1.04 to 12.9-fold compared to the galactose oxidase variant M3-5. In one example, the modified galactose oxidase disclosed herein, reduced enantioselectivity by at least 1.1-fold compared to the galactose oxidase variant M3-5. In one example, the modified galactose oxidase disclosed herein, reduced enantioselectivity by at least 2-fold compared to the galactose oxidase variant M3-5. In one example, the modified galactose oxidase disclosed herein, reduced enantioselectivity by at least 3-fold compared to the galactose oxidase variant M3-5. In one example, the modified galactose oxidase disclosed herein, reduced enantioselectivity by at least 4-fold compared to the galactose oxidase variant M3-5. In one example, the modified galactose oxidase disclosed herein, reduced enantioselectivity by at least 5-fold compared to the galactose oxidase variant M3-5. In one example, the modified galactose oxidase disclosed herein, reduced enantioselectivity by at least 6-fold compared to the galactose oxidase variant M3-5. In one example, the modified galactose oxidase disclosed herein, reduced enantioselectivity by at least 7-fold compared to the galactose oxidase variant M3-5. In one example, the modified galactose oxidase disclosed herein, reduced enantioselectivity by at least 8-fold compared to the galactose oxidase variant M3-5. In one example, the modified galactose oxidase disclosed herein, reduced enantioselectivity by at least 9-fold compared to the galactose oxidase variant M3-5. In one example, the modified galactose oxidase disclosed herein, reduced enantioselectivity by at least 10-fold compared to the galactose oxidase variant M3-5. In one example, the modified galactose oxidase disclosed herein, reduced enantioselectivity by at least 11-fold compared to the galactose oxidase variant M3-5. In one example, the modified galactose oxidase disclosed herein, reduced enantioselectivity by at least 12-fold compared to the galactose oxidase variant M3-5. In one specific example, the modified galactose oxidase disclosed herein, reduced enantioselectivity by 12.9-fold compared to the galactose oxidase variant M3-5. In one specific example, the GOh1XXX modified galactose oxidase disclosed herein, has minimum reduced enantioselectivity of 0.8 R/S ratio from M3-5 experimental value of 20.6 R/S ratio (for substrate 128—alpha-tetralol). In one example, the modified galactose oxidase disclosed herein, reduced enantioselectivity by about 1.15 to 1.59-fold compared to the galactose oxidase variant GOh1052. In one example, the modified galactose oxidase disclosed herein, reduced enantioselectivity by at least 1.2-fold compared to the galactose oxidase variant GOh1052. In one example, the modified galactose oxidase disclosed herein, reduced enantioselectivity by at least 1.3-fold compared to the galactose oxidase variant GOh1052. In one example, the modified galactose oxidase disclosed herein, reduced enantioselectivity by at least 1.4-fold compared to the galactose oxidase variant GOh1052. In one example, the modified galactose oxidase disclosed herein, reduced enantioselectivity by at least 1.5-fold compared to the galactose oxidase variant GOh1052. In one specific example, the modified galactose oxidase disclosed herein, reduced enantioselectivity by 1.59-fold compared to the galactose oxidase variant GOh1052. In one specific example, the GOh2XXX and GOh3XXX modified galactose oxidase disclosed herein, has minimum reduced enantioselectivity of 1.04 R/S ratio from M3-5 (for substrate 128—alpha-tetralol) or from GOh1052 (for substrate 152-1-phenyl-1-butanol).

In one aspect, the present disclosure refers to a polynucleotide sequence encoding the modified galactose oxidase disclosed herein.

In one example, the present disclosure refers to the polynucleotide sequence disclosed herein, wherein the polynucleotide sequence comprises at least 85%, 86%, 87%, 88%, 89%, 90%, 91%, 92%, 93%, 94%, 95%, 96%, 97%, 98%, or 99% or more sequence identity to SEQ ID NO: 5, wherein SEQ ID NO: 5 is a polynucleotide sequence of the galactose oxidase variant M3-5, wherein the polynucleotide sequence disclosed herein comprises at least one mutation at one or more positions which leads to at least one mutation in the polypeptide sequence of the modified galactose oxidase disclosed herein.

In one example, the present disclosure refers to the polynucleotide sequence disclosed herein, wherein the polynucleotide is codon optimized, wherein optionally the polynucleotide is codon optimized for protein expression in Escherichia coli. In one example, the polynucleotide is codon optimized for protein expression in other types of cells known in the art, such as yeast cells, mammalian cells or other suitable cells known in the art. In one example, the polynucleotide is codon optimized for protein expression in yeast cells such as Saccharomyces cerevisiae and Pichia pastoris. In one example, the polynucleotide is codon optimized for protein expression in mammalian cells such as Chinese Hamster Ovary (CHO) cells. The term “codon optimization” as used herein, refers to methods to improve codon composition of a polynucleotide without altering the amino acid sequence, since it is known in the art that most amino acids can be encoded by more than one codon.

In one example, the present disclosure refers to the polynucleotide sequence disclosed herein, wherein the polynucleotide sequence is further linked to a control sequence. In one example, the control sequence is a promoter sequence or a termination sequence. In one example, the promoter sequence is a lac promoter sequence, CMV promoter sequence, SV40 promoter sequence, PBAD promoter sequence, GAL promoter sequence, AOX1 promoter sequence, or other promoter sequences that are well-known in the art. In one example, the PBAD promoter can be used for controlling gene expression in Escherichia coli (E. coli). In one example, the GAL promoter can be used for controlling gene expression in Saccharomyces cerevisiae (S. cerevisiae). In one example, the AOX1 promoter can be used for controlling gene expression in Pichia pastoris (P. pastoris). In one specific example, the promoter sequence is a lac promoter sequence. The term “control sequence” as used herein refers to a regulatory sequence or regulatory element within a polynucleotide sequence that regulates gene expression. Examples of “control sequences” that are well-known in the art are promoters, enhancers, repressors, and terminators.

In one example, the present disclosure refers to the polynucleotide sequence disclosed herein, wherein the polynucleotide comprises an odd numbered sequence of SEQ ID NOS: 7-298. In one example, the present disclosure refers to the polynucleotide sequence disclosed herein, wherein the polynucleotide comprises an odd numbered sequence of SEQ ID NOS: 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, or 297. In one example, the present disclosure refers to the polynucleotide sequence disclosed herein, wherein the polynucleotide comprises the sequence of SEQ ID NO: 7. In one example, the present disclosure refers to the polynucleotide sequence disclosed herein, wherein the polynucleotide comprises the sequence of SEQ ID NO: 9. In one example, the present disclosure refers to the polynucleotide sequence disclosed herein, wherein the polynucleotide comprises the sequence of SEQ ID NO: 11. In one example, the present disclosure refers to the polynucleotide sequence disclosed herein, wherein the polynucleotide comprises the sequence of SEQ ID NO: 13. In one example, the present disclosure refers to the polynucleotide sequence disclosed herein, wherein the polynucleotide comprises the sequence of SEQ ID NO: 15. In one example, the present disclosure refers to the polynucleotide sequence disclosed herein, wherein the polynucleotide comprises the sequence of SEQ ID NO: 17. In one example, the present disclosure refers to the polynucleotide sequence disclosed herein, wherein the polynucleotide comprises the sequence of SEQ ID NO: 19. In one example, the present disclosure refers to the polynucleotide sequence disclosed herein, wherein the polynucleotide comprises the sequence of SEQ ID NO: 21. In one example, the present disclosure refers to the polynucleotide sequence disclosed herein, wherein the polynucleotide comprises the sequence of SEQ ID NO: 31. In one example, the present disclosure refers to the polynucleotide sequence disclosed herein, wherein the polynucleotide comprises the sequence of SEQ ID NO: 41. In one example, the present disclosure refers to the polynucleotide sequence disclosed herein, wherein the polynucleotide comprises the sequence of SEQ ID NO: 51. In one example, the present disclosure refers to the polynucleotide sequence disclosed herein, wherein the polynucleotide comprises the sequence of SEQ ID NO: 61. In one example, the present disclosure refers to the polynucleotide sequence disclosed herein, wherein the polynucleotide comprises the sequence of SEQ ID NO: 71. In one example, the present disclosure refers to the polynucleotide sequence disclosed herein, wherein the polynucleotide comprises the sequence of SEQ ID NO: 81. In one example, the present disclosure refers to the polynucleotide sequence disclosed herein, wherein the polynucleotide comprises the sequence of SEQ ID NO: 91. In one example, the present disclosure refers to the polynucleotide sequence disclosed herein, wherein the polynucleotide comprises the sequence of SEQ ID NO: 101. In one example, the present disclosure refers to the polynucleotide sequence disclosed herein, wherein the polynucleotide comprises the sequence of SEQ ID NO: 151. In one example, the present disclosure refers to the polynucleotide sequence disclosed herein, wherein the polynucleotide comprises the sequence of SEQ ID NO: 201. In one example, the present disclosure refers to the polynucleotide sequence disclosed herein, wherein the polynucleotide comprises the sequence of SEQ ID NO: 251. In one example, the present disclosure refers to the polynucleotide sequence disclosed herein, wherein the polynucleotide comprises the sequence of SEQ ID NO: 297.

In one aspect, the present disclosure refers an expression vector comprising the polynucleotide sequence disclosed herein. In one example, the expression vector is selected from the group consisting of a plasmid and a viral vector. Suitable expression vectors for production of proteins (such as enzymes, such as the modified galactose oxidase of the present disclosure) are well known to those skilled in the art. Examples of plasmid vectors include pET28a, pcDNA3.1, pGEX, pCMV, pYES, and pGAPZ. In one example, the pYES plasmid vector is designed for protein expression in Saccharomyces cerevisiae (S. cerevisiae). In one example, the pGAPZ plasmid vector is designed for protein expression in Pichia pastoris (P. pastoris). In one specific example, the expression vector is a pET28a plasmid vector. Examples of viral vectors include Adeno-Associated Viral Vector (AAV) and lentiviral vector. In one example, the expression vector comprising the polynucleotide sequence disclosed herein may be vectors generally used for the production of proteins/enzymes which are known to those skilled in the art.

In one aspect, the present disclosure refers to a host cell comprising the polynucleotide sequence disclosed herein or the expression vector disclosed herein. In one example, the present disclosure refers to a host cell comprising the polynucleotide sequence disclosed herein. In one example, the present disclosure refers to a host cell comprising the expression vector disclosed herein. In one example, the host cell disclosed herein is a prokaryotic cell or eukaryotic cell. In one example, the host cell is a prokaryotic cell. In one specific example, the prokaryotic cell is Escherichia coli. In one example, the host cell comprising the polynucleotide sequence disclosed herein or the expression vector disclosed herein is E. coli BL21(DE3). In one example, the host cell comprising the polynucleotide sequence disclosed herein or the expression vector disclosed herein may be cells generally used for the production of proteins/enzymes which are known to those skilled in the art.

In one aspect, the present disclosure refers to a method of producing a modified galactose oxidase, comprising culturing the host cell disclosed herein under suitable culture conditions such that the modified galactose oxidase is produced. In one example, the method comprises culturing the host cell disclosed herein in a culture medium. In one example, the culture medium is lysogeny broth (LB) medium supplemented with kanamycin, or any other suitable culture medium known in the art. In one example, the method of producing the modified galactose oxidase disclosed herein, further comprises recovering the modified galactose oxidase from the culture and/or the host cell. In one example, the method of producing the modified galactose oxidase disclosed herein, further comprises recovering the modified galactose oxidase from the culture. In one example, the method of producing the modified galactose oxidase disclosed herein, further comprises recovering the modified galactose oxidase from the host cell. In one example, the method of producing the modified galactose oxidase disclosed herein, further comprises recovering the modified galactose oxidase from the culture and host cell. In one example, the method of recovering the modified galactose oxidase disclosed herein from the culture and host cell are commonly used methods of protein recovery from culture and host cells which are well-known to those skilled in the art. In one example, the modified galactose oxidase disclosed herein is recovered from the culture and host cell through cell harvesting by centrifugation, where the proteins from the culture medium are separated based on density differences. In one example, the modified galactose oxidase disclosed herein is recovered from the culture and host cell through cell harvesting by filtration, whereby culture media are filtered to separate host cells from the culture media using membranes filters. In one example, the modified galactose oxidase disclosed herein is recovered from the culture and host cell through cell lysis/disruption by mechanical or chemical disruption of host cells to disrupt cell walls/membranes and release intracellular proteins. In one example, the method of producing the modified galactose oxidase disclosed herein, further comprises purifying the modified galactose oxidase. In one example, the method of purifying the modified galactose oxidase disclosed herein is by affinity chromatography, using specific affinity tags such as His-tag and GST-tag. In one example, the method of purifying the modified galactose oxidase disclosed herein is by ion exchange chromatography, whereby proteins are separated based on charge differences. In one example, the method of purifying the modified galactose oxidase disclosed herein is by size exclusion chromatography, whereby proteins are separated based on their size and molecular weight. In one specific example, the method of purifying the modified galactose oxidase disclosed herein is by affinity chromatography, using His-tag. In one example, the method of producing and purifying the modified galactose oxidase disclosed herein may be methods generally used for the production and purification of proteins, such as enzymes, which are known to those skilled in the art.

In one aspect, the present disclosure refers to a method of producing a ketone from a secondary alcohol, comprising contacting the secondary alcohol with the modified galactose oxidase disclosed herein under suitable conditions for an oxidation reaction. In one example, the secondary alcohol is a bulky secondary alcohol or unactivated secondary alcohol. In one example, the secondary alcohol is a bulky secondary alcohol which comprises one or more bulky substituents. In one example, the unactivated secondary alcohol is an aliphatic secondary alcohol without a benzene ring in the alpha position adjacent to the alcohol group.

In one example, the secondary alcohol comprises the formula R¹R²CHOH, wherein R¹ and R² are independently selected from the group consisting of cycloalkyl, aryl, heteroaryl, alkyl, alkylene, alkynyl, haloalkyl, carboxyl, and carbonyl, wherein the cycloalkyl, aryl, heteroaryl, alkyl, alkylene, alkynyl, haloalkyl, carboxyl, and carbonyl is optionally substituted with one or more substituents selected from the group consisting of alkyl, halogen, alkyl halide, alkylene, alkynyl, alkoxy, cyano, oxo, nitro, amino, thiol, carboxyl, ester and hydroxyl. In one example, the secondary alcohol is selected from the group consisting of:

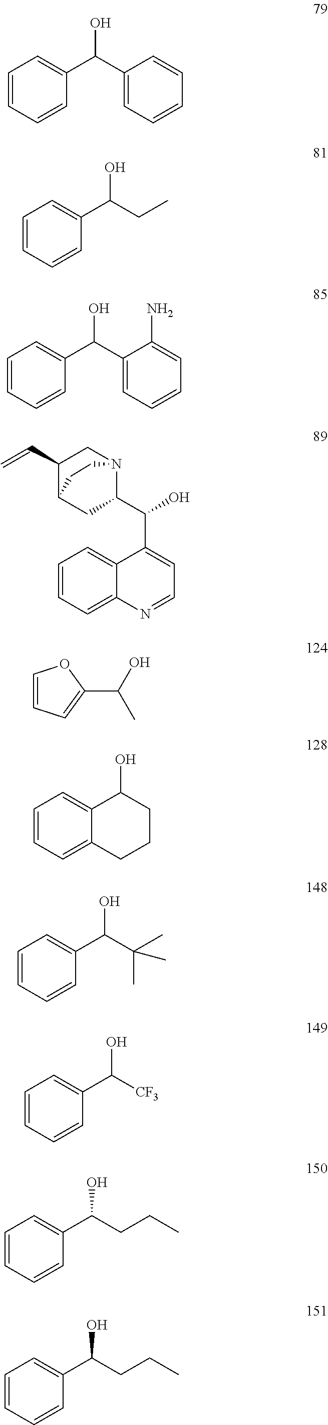

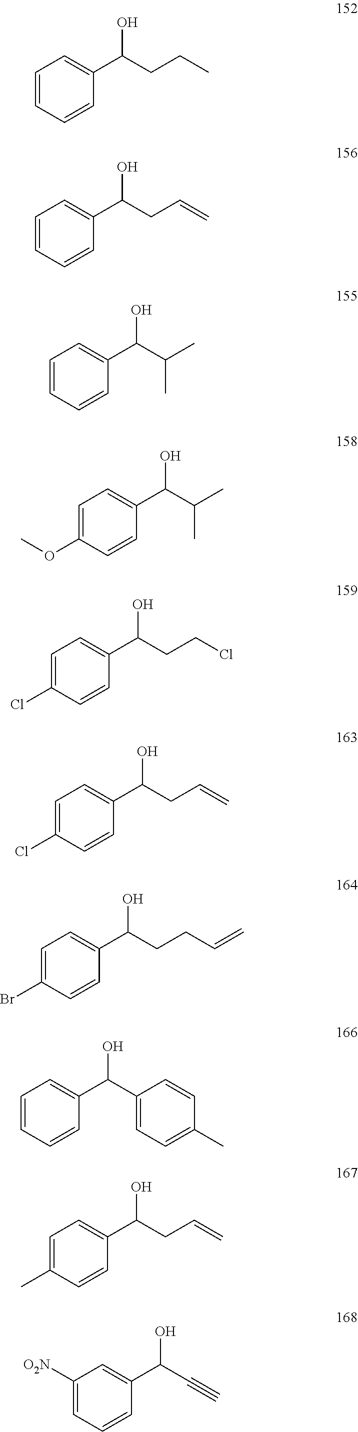

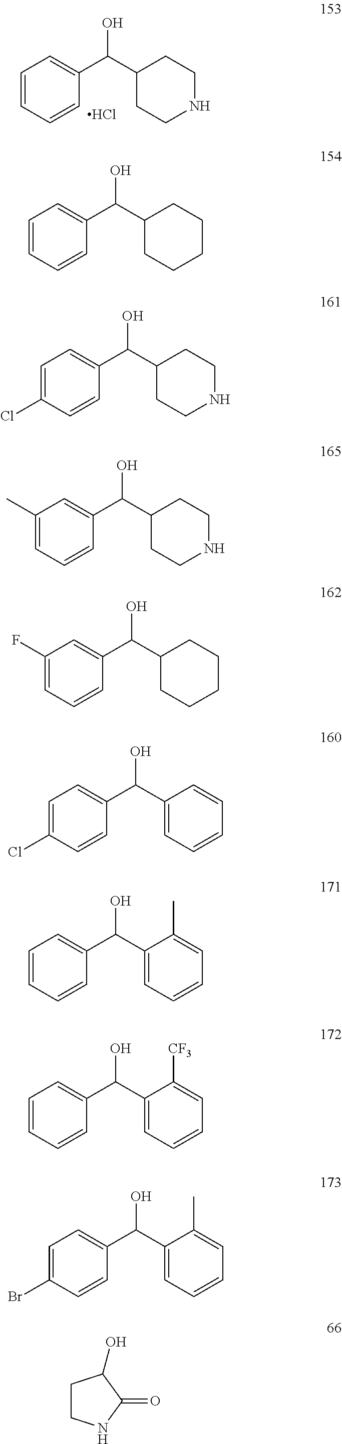

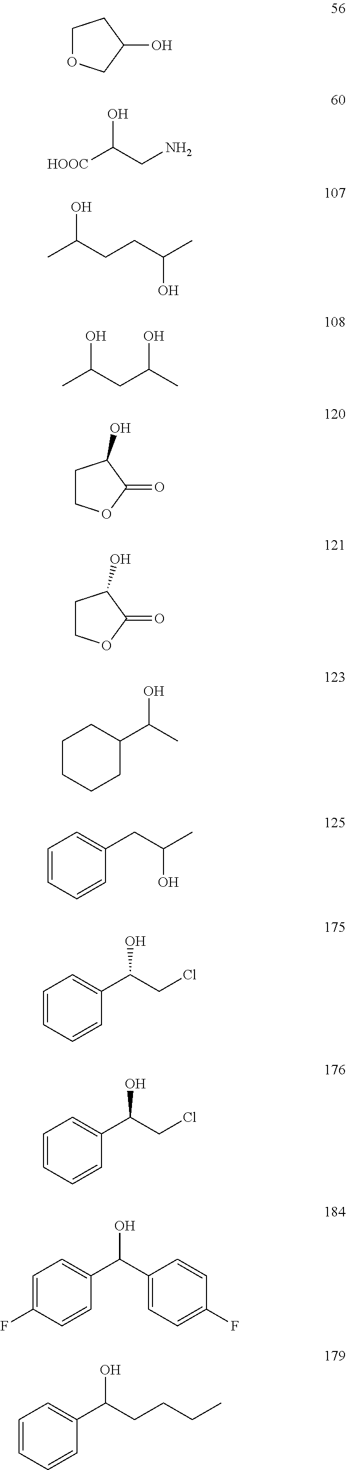

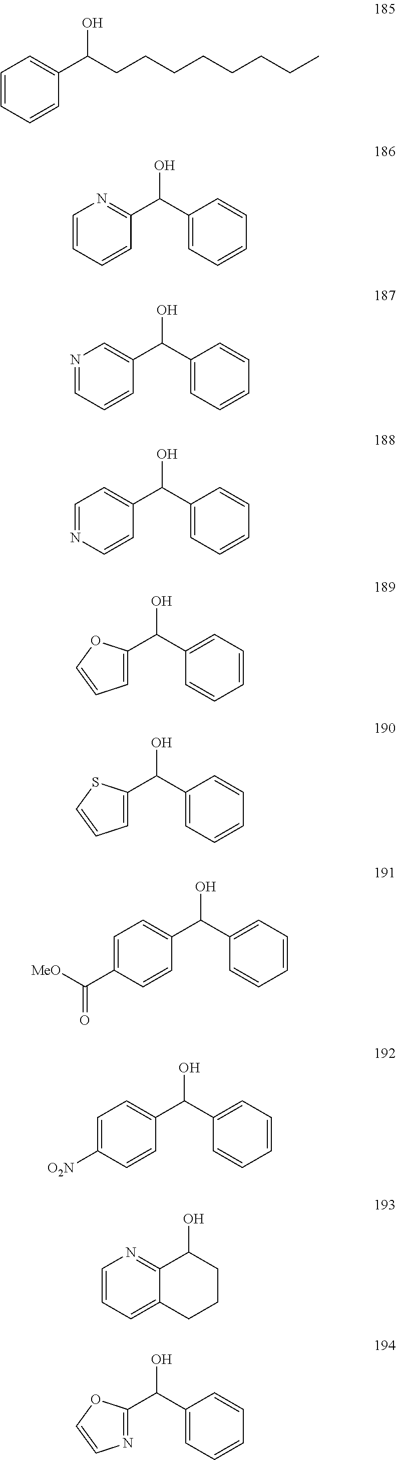

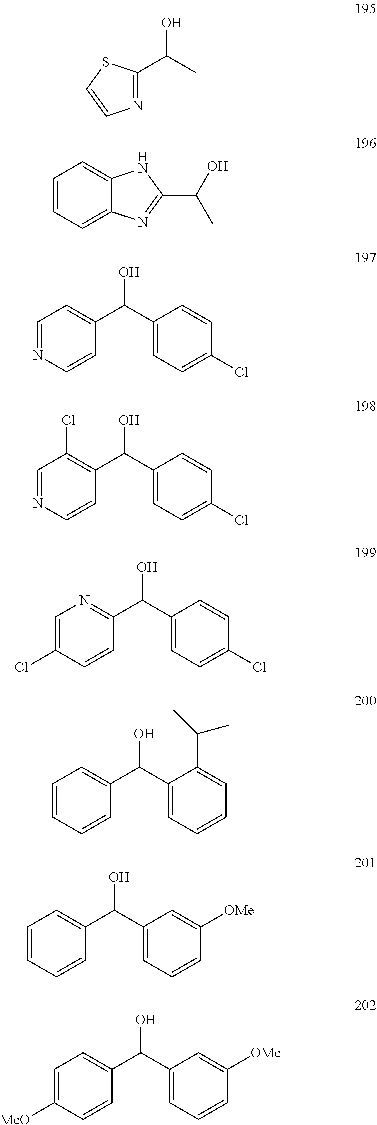

In one example, the secondary alcohol is chiral or non-chiral. In one example, the secondary alcohol is chiral. In one example, the secondary alcohol is non-chiral. In one example, the chiral secondary alcohol is present as (S)-enantiomer, (R)-enantiomer, or a mixture of (S)- and (R)-enantiomers. In one example, the chiral secondary alcohol is present as a racemic mixture.

In one example, the present disclosure refers to a method of producing a ketone from a secondary alcohol, comprising contacting the secondary alcohol with the modified galactose oxidase disclosed herein, wherein the contacting step is performed at a temperature of 25° C., 30° C., 35° C., 40° C., 45° C., 50° C., 55° C. or more. In one example, the contacting step is performed at a temperature of 25° C. In one example, the contacting step is performed at a temperature of 30° C. In one example, the contacting step is performed at a temperature of 35° C. In one example, the contacting step is performed at a temperature of 40° C. In one example, the contacting step is performed at a temperature of 45° C. In one example, the contacting step is performed at a temperature of 50° C. In one example, the contacting step is performed at a temperature of 55° C. In one example, the contacting step is performed at a temperature higher than 55° C. In one example, the contacting step refers to a step where the substrate and the enzyme are first mixed, before the oxidation reaction occurs. In one example, the contacting step is performed at a temperature of 25° C., 30° C., 35° C., 40° C., 45° C., 50° C., 55° C. or more, wherein optionally the temperature is 35° C. In one example, the oxidation reaction can be performed at lower temperatures than 40° C. and can be conducted at 25° C. In some examples, certain galactose oxidase mutants have shown enzymatic activity even after a thermal challenge at 55° C. Optimal temperature for the reaction depends on, for example, enzyme variant, substrate as well as which output variable. Taking GOh1052 variant with substrate 152 (1-phenyl-1-butanol) as an example, although 40° C. is the optimal temperature for fastest initial rate of reaction, 35° C. is preferred over a 24-hour reaction period as it results in higher overall yields (72% at 35° C. vs 55% at 40° C.).

In one example, the method of producing a ketone from a secondary alcohol disclosed herein, further comprises converting the secondary alcohol into an active pharmaceutical ingredient (API). The term “active pharmaceutical ingredient” as used herein refers to a biologically active component of a pharmaceutical drug responsible for the therapeutic effect of the drug.

In one aspect, the present disclosure refers to a kit for use in the method disclosed herein, wherein the kit comprises the modified galactose oxidase disclosed herein. In one example, the kit disclosed herein comprises one or more of the following: (a) one or more reaction buffers; (b) one or more reaction vessels, wherein optionally the reaction vessel is a HPLC vial; (c) one or more reaction solvents, wherein optionally the reaction solvent is 5% v/v dimethyl sulfoxide in 100 mM sodium phosphate buffer with a pH of 7; (d) one or more secondary alcohol substrates; (e) one or more co-enzymes to break down the by-product of the oxidation reaction, wherein optionally the co-enzyme is a horseradish peroxidase that breaks down the H₂O₂; and (f) instructions for performing the method disclosed herein. In one example, the one or more reaction buffer is Jones Reagent. In one example, the one or more reaction buffer is PCC (Pyridinium chlorochromate). In one example, the one or more reaction buffer contains an enzyme capable of catalysing the conversion of secondary alcohols to ketones. In one example, the enzyme is alcohol dehydrogenase. In one example, the one or more reaction buffer contains an enzyme and cofactors. In one example, the enzyme is alcohol dehydrogenase and the cofactor is NAD⁺ (nicotinamide adenine dinucleotide) or NADP⁺ (nicotinamide adenine dinucleotide phosphate). In some examples, the reagents in the kit as described herein may be provided in separate containers comprising the components independently distributed in one or more containers.

### Examples

### Methods

Galactose Oxidase Expression in E. coli and Purification

The wildtype galactose oxidase gene from Fusarium graminearum was synthesized (Twist Bioscience) and cloned into the expression vector pET28a (Novagen) using the NdeI and XhoI restriction sites. Eight additional mutations (S33P, M93V, G218E, W313F, R353M, Q429T, V517A, N558D) were incorporated into the wildtype galactose oxidase gene via splicing by overlap extension (SOE) method to produce GOh1001b variant. The GOh1001b plasmid was then transformed into E. coli BL21(DE3) strain. The E. coli containing the plasmid was grown in 250 mL lysogeny broth (LB) medium with 50 μg/mL kanamycin at 37° C. until an absorbance of 0.5 at 600 nm was reached. Overexpression was induced by adding 0.1 mM isopropyl β-D-1-thiogalactopyranoside (IPTG) and incubation was continued at 16° C. for 20 hours. Cells were harvested by centrifugation (2,450×g for 15 minutes). The cell pellet was resuspended in 12 mL of 100 mM sodium phosphate buffer, pH 7.0 and the cells were lysed using a cell disruptor (Constant Systems) at 40 kPSI. The lysate was then centrifuged, and the clarified supernatant was added to TALON metal affinity resin (Clontech). The supernatant-resin mixture was incubated at 4° C. with rotation for 2 hours. The His-tagged protein bound resin was washed with binding buffer (100 mM sodium phosphate buffer, pH 7.0, 300 mM NaCl, 5 mM imidazole) and eluted with elution buffer (100 mM sodium phosphate buffer, pH 7.0, 300 mM NaCl, 250 mM imidazole). The elution was desalted using PD-10 desalting column (GE Healthcare) in 100 mM sodium phosphate buffer, pH 7.0. The protein concentration was measured using Pierce™ BCA Protein Assay kit (Thermo Fisher Scientific) against diluted albumin (BSA) standards as provided inside the kit. The purified protein was then diluted to 400 μg/mL and activated with 1 mM copper sulphate at 25° C. for 15 minutes.

### Codon Optimization for GO01b Variant

Synthesized gene was ordered from Twist Bioscience. Utilizing their website tool, amino acid sequence was entered, and desired codon usage table selected (E. coli). Twist Bioscience's codon optimization algorithm subsequently provided the DNA nucleotide sequence. Algorithm results are not reproducible as identical amino acid sequences keyed in twice will return different DNA nucleotide sequences.

### Library Construction and New Variant Construction

Round 1: Saturation mutagenesis (NNK) at the actives sites was performed on GOh1001b variant via SOE method to generate libraries for high throughput (HTP) screening. Beneficial mutations from HTP screening were combined via SOE method.
Round 2: Site saturation mutagenesis libraries on GOh1052 variant were ordered from either Twist Bioscience or GenScript with codon usage for E. coli expression at each mutated position only.
Round 3: Beneficial mutations from Round 2 were combined via SOE method on GOh1052 variant. For combinatorial library on GOh1052 variant, oligos with wildtype DNA codon and mutation DNA codon at each position were ordered and pooled together with equal ratio to give a theoretical 50% rate of mutation incorporation. Combinatorial library was constructed via SOE method.

### High Throughput Growth and Lysis

For each NNK library in round 1, 84 colonies for variants (32 codons, ˜93% coverage), six (6) colonies for GOh1001b variant, and six (6) colonies for negative control (pET28a) were inoculated into a 96-well plate. For round 2 libraries, 19 colonies per position (i.e., 19 other amino acid residues from wildtype residue), six (6) colonies for GOh1052 variant, and six (6) colonies for negative control (pET28a) were inoculated into a 96-well plate. For round 3 combinatorial library, 84 colonies for variants, six (6) colonies for GOh1052 variant, and six (6) colonies for negative control (pET28a) were inoculated into a 96-well plate (for five (5) plates, ˜95% coverage) Individual colony was inoculated in 180 μL LB medium containing kanamycin (50 μg/mL) and cultured overnight at 37° C. 20 μL of cultures were transferred to 380 μL LB medium containing 50 μg/mL kanamycin and grown until an absorbance of 0.5 at 600 nm was reached. Overexpression was induced by adding 0.1 mM IPTG and incubation was continued at 16° C. for 20 hours. Cells were harvested by centrifugation (2,182×g for 10 min at 4° C.). The cell pellets were lysed with 250 μL lysis buffer (100 mM sodium phosphate buffer, pH 7.0, 0.1% Triton-X, 1 mM copper sulphate) at room temperature for 2 hours at speed 6.5 on titer plate shaker (Barnstead). The lysates were obtained by centrifugation (2,182×g for 20 min at 4° C.).

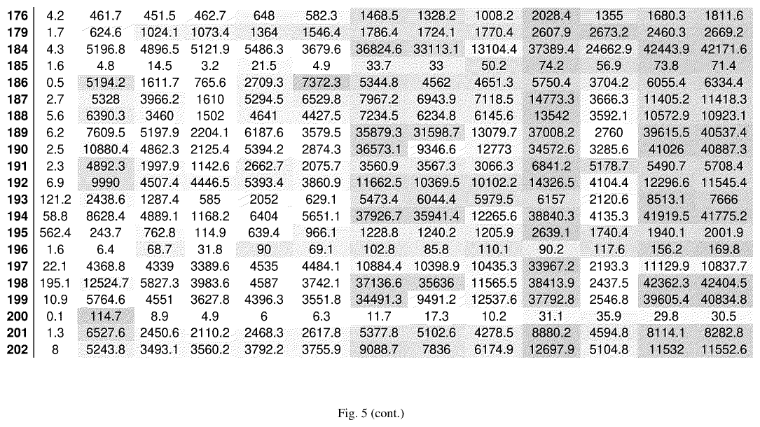

### ABTS-HRP* Colorimetric Assay (FIG. 5)

HTP colorimetric assay conditions: Substrate (1 mM in DMSO), ABTS (2.5 mM in water), HRP (Toyobo, 25 μg/mL), and galactose oxidase lysates (25 μL) in a final volume of 200 μL with final concentration of dimethylsulfoxide (DMSO) at 5% v/v in 100 mM sodium phosphate buffer (pH 7.0). HTP colorimetric assay was carried out using 96-well clear shallow well plates at room temperature and read at 405 nm using a microplate reader (Tecan Infinite M200 Pro) for the following timepoints: every 2 minutes for 1 hour, every 10 minutes for next 2 hours, every 30 minutes for next 5 hours, every 2 hours for next 16 hours for a total of 24 hours.

Focused secondary alcohol substrate panel colorimetric assay: Identical conditions to HTP colorimetric assay except with galactose oxidase purified protein (20 μg/mL) instead of galactose oxidase lysates. Better performing variants against secondary alcohol substrates were selected based on the following formula (maximum absorbance×initial velocity). *ABTS refers to 2,2′-azino-bis(3-ethylbenzothiazoline-6-sulfonic acid), HRP refers to Horseradish peroxidase.

### F217 Fitness Landscape (Enantiomeric Bias)

HTP enzymatic reaction conditions: Substrate 128 (1 mM), HRP (25 μg/mL), and galactose oxidase purified protein (100 μg/mL) in a final volume of 200 μL with final concentration of DMSO at 5% v/v in 100 mM sodium phosphate buffer (pH 7.0). HTP enzymatic reaction was carried out using 96-well deepwell plates with breathable seal at 25° C., 220 rpm. Reaction was stopped at 24 hours via addition of methanol (200 μL). The precipitated protein was then removed by centrifugation (2,182×g for 20 minutes). 10 μL of the reaction mixture was sampled for HPLC-UV analysis.

### Thermostability Assay

Enzyme heat treatment conditions: Purified enzyme (60 μL, 400 μg/mL) was incubated at 55° C. for 30 minutes in PCR strip tubes using Bio-Rad PTC-200 Thermal Cycler. The heat-treated enzymatic solution was transferred to 1.5 mL tubes and the precipitated protein was removed by centrifugation (20,238×g for 5 min at 4° C.). 50 μL of the heat-treated enzymatic solution was transferred to a 96 deep well plate for activity assay. Activity assay: Substrate 128 (1 mM), HRP (25 μg/mL), and heat-treated galactose oxidase purified protein (100 μg/mL) in a final volume of 200 μL with final concentration of DMSO at 5% v/v in 100 mM sodium phosphate buffer (pH 7.0). Reactions were carried out in 96 deepwell plates with breathable seal at 25° C., 220 rpm. Reactions were stopped at 24 h via addition of methanol (200 μL). The precipitated protein was then removed by centrifugation (2,182×g for 20 minutes at 4° C.). 10 μL of the reaction mixture was sampled for HPLC-UV analysis. The full experimental materials include:
Suitable reaction vessel (i.e. 2 mL HPLC vials were used)
5% v/v Dimethyl sulfoxide in 100 mM sodium phosphate buffer (pH=7) as reaction solvent (200 μL)
Secondary alcohol substrate (1 mM)
Horseradish peroxidase (25 μg/mL) as a co-enzyme to breakdown H₂O₂ that is produced as a by-product of the oxidation reaction
Galactose oxidase protein (40 μg/mL)
Sufficient atmospheric oxygen content.

### Enzyme Kinetics

Kinetics colorimetric assay conditions for substrate 128: Substrate 128 (varying concentrations from 2.5 to 30 mM), ABTS (2.5 mM), HRP (25 μg/mL) and galactose oxidase purified protein (amount is variant dependent) in a final volume of 200 μL with final concentration of DMSO at 5% v/v in 100 mM sodium phosphate buffer (pH 7.0). Kinetic parameters were obtained by the best-fit model of initial velocity against substrate concentrations based on Michaelis-Menten equation using GraphPad Prism 8 (GraphPad Software).

Amount of galactose oxidase purified protein used: GOh1001b (100 μg/mL), GOh1021 (10 μg/mL), GOh1036 (10 μg/mL), GOh1052 (5 μg/mL)

### High-Performance Liquid Chromatography (HPLC)

### HPLC Validation

Purified protein reaction conditions: Substrate (1 mM), HRP (25 μg/mL), and galactose oxidase purified protein (100 μg/mL) in a final volume of 200 μL with final concentration of DMSO at 5% v/v in 100 mM sodium phosphate buffer (pH 7.0). Purified protein reaction was carried out in sealed 2 mL HPLC vials at 25° C., 220 rpm. Reaction was stopped at 24 hours via addition of methanol (200 μL). The precipitated protein was then removed by centrifugation (20,238×g for 1 minute). 10 μL of the reaction mixture was sampled for HPLC-UV analysis.

40° C. heated activity assay conditions: The conditions are identical to the purified protein reaction conditions described above except that the reactions are conducted at 40° C. temperature for a reaction period of 6 hours.

### HPLC Analysis

HPLC analysis was carried out using Shimadzu Prominence system UFLC or Agilent HPLC 1200 Infinity series paired with CTC Analytics HTS PAL LC Autosampler.

Mobile phase A: Water; B: Acetonitrile. Column: Phenomenex, Luna® 5 μm C18(2) 100 Å, LC Column 150×4.6 mm, 1 mL/minute flow rate

Standards (200 μL) of 1 mM substrate and 1 mM product for each compound were prepared with 5% DMSO v/v in 100 mM sodium phosphate buffer (pH 7.0) then diluted with methanol (200 μL) before being run with each sample for quantification according to the following formula:

$$HPLC Yield \% = \frac{Peak Integral ( Sample )}{Peak Integral ( Standard )}$$

### HRP-ABTS Colorimetric Assay

### HRP-ABTS Colorimetric Assay Screening Data

Colorimetric activities were assessed calculated from two parameters measured via 405 nm absorbance data gathered by microplate reader (Tecan) according to the following formula:

$$Colorimetric Activity = Rate \times Maximum Absorbance \times 100$$

where,
Colorimetric Activity=measure of enzymatic activity
Rate=initial rate of increase in absorbance in the first 8 minl
Maximum Absorbance=maximum absorbance recorded over the 24 h reaction period

TABLE 1
Table of galactose oxidase mutations investigated
in the present disclosure

| | Position of mutation, numbered based on the protein |
|---|---|
| Mutation | sequence of the M3-5 variant of galactose oxidase |
| A24H | 24 |
| A24N | 24 |
| A26H | 26 |
| A26R | 26 |
| I28F | 28 |
| I28M | 28 |
| I28R | 28 |
| Q45D | 45 |
| Q45E | 45 |
| Q45N | 45 |
| E49K | 49 |
| F65P | 65 |
| N69D | 69 |
| N69E | 69 |
| N69F | 69 |
| N69H | 69 |
| N69L | 69 |
| N69M | 69 |
| N69T | 69 |
| N69W | 69 |
| N69Y | 69 |
| W104G | 104 |
| W104N | 104 |
| S114N | 114 |
| S114R | 114 |
| T117D | 117 |
| T153K | 153 |
| T153R | 153 |
| G157N | 157 |
| S172E | 172 |
| S173K | 173 |
| S173R | 173 |
| T175E | 175 |
| T175H | 175 |
| T175I | 175 |
| T175K | 175 |
| V193T | 193 |
| P194C | 194 |
| A195S | 195 |
| A195T | 195 |
| A195V | 195 |
| A197C | 197 |
| A197G | 197 |
| A198I | 198 |
| T202D | 202 |
| T202I | 202 |
| T202K | 202 |
| T202Q | 202 |
| N214R | 214 |
| N214S | 214 |
| F217A | 217 |
| F217C | 217 |
| F217D | 217 |
| F217E | 217 |
| F217G | 217 |
| F217H | 217 |
| F217I | 217 |
| F217K | 217 |
| F217L | 217 |
| F217M | 217 |
| F217N | 217 |
| F217P | 217 |
| F217Q | 217 |
| F217R | 217 |
| F217S | 217 |
| F217T | 217 |
| F217V | 217 |
| F217Y | 217 |
| E218F | 218 |
| E218K | 218 |
| E218P | 218 |
| E218Y | 218 |
| S220T | 220 |
| I224W | 224 |
| L226Q | 226 |
| L226V | 226 |
| D239E | 239 |
| D239P | 239 |
| D239Q | 239 |
| R240G | 240 |
| F250E | 250 |
| F250Y | 250 |
| N259T | 259 |
| T265S | 265 |
| N268W | 268 |
| D269N | 269 |
| I284S | 284 |
| I284Y | 284 |
| G294A | 294 |
| F313R | 313 |
| S314C | 314 |
| Y325F | 325 |
| N341W | 341 |
| A346L | 346 |
| K348S | 348 |
| Y352T | 352 |
| Y352W | 352 |
| M353F | 353 |
| N356K | 356 |
| N356R | 356 |
| N356W | 356 |
| K389I | 389 |
| A401D | 401 |
| T429W | 429 |
| T447A | 447 |
| I485A | 485 |
| I485K | 485 |
| I485R | 485 |
| F487R | 487 |
| L537G | 537 |
| C538K | 538 |
| C538R | 538 |
| F546M | 546 |
| S559P | 559 |
| S576D | 576 |
| Y599M | 599 |
| Q662D | 662 |
| Q662P | 662 |

### Exemplary Minimum Fold Changes

The following exemplary minimum fold changes are to be considered:
For GOh1XXX variant activity (Table 2)—1.5-fold more than M3-5 for Substrate 128 (alpha-tetralol)
For GOh2XXX and GOh3XXX variant activity (Tables 3 and 4-1.04-fold more than GOh1052 for substrate 152 (1-phenyl-1-butanol) indicated by “Y” in “Activity (HPLC)” column in Table 6.
For GOhXXXX variant activity (Table 2-1.1-fold better than M3-5 for 1 or more substrates in the 64-substrate panel in FIG. 1.
For GOh1XXX protein expression (Table 14)—1.06-fold better than M3-5; For GOh1XXX solubility (Table 16)—1.13-fold better than M3-5; and thermal stability (Table 12)—1.52-fold better than M3-5
For GOh2XXX and GOh3XXX protein expression (Table 13)—1.01-fold better than GOh1052; solubility (Table 15)—1.02-fold better than GOh1052; and thermal stability (Table 11)—1.01-fold better than GOh1052
For GOh1XXX enantioselectivity (Table 18—minimum reduction of 0.8 R/S ratio from M3-5 experimental value of 20.6 R/S ratio (for substrate 128—alpha-tetralol)
For GOh2XXX and GOh3XXX enantioselectivity (Tables 17 and 18-minimum 1.04-fold change reduction in R/S ratio from M3-5 (for substrate 128—alpha-tetralol) or from GOh1052 (for substrate 152-1-phenyl-1-butanol)
For ease of comparison, interpretation of reduced enantioselectivity is conveyed as absolute units as a ratio in conversion of (R)- and (S)-enantiomers of the substrate. Higher R/S ratios indicate higher enantioselectivity for (R)-enantiomers while R/S ratios closer to 1 indicate reduced enantioselectivity, with 1 R/S ratio being no enantioselectivity.

TABLE 2
The colorimetric activity of purified galactose oxidase enzymes
activity profile benchmarked against M3-5 backbone.

| | | Mutations | Activity Fold |
|---|---|---|---|
| Mutant No. | Substrate | (on M3-5) | Change |
| M3-5 | 66, 120, 121, 128, 166 | backbone | 1.0 |
| GOh1008 | 66 | S314C | 19.9 |
| GOh1017 | 66 | F217Q | 8.4 |
| GOh1018 | 66 | F217N | 3.1 |
| GOh1042 | 66 | F217M | 3.0 |
| GOh1039 | 66 | F217G | 2.2 |
| GOh1020 | 66 | F217S | 2.0 |
| GOh1036 | 66 | F217A | 1.3 |
| GOh1006 | 66 | M353F | 1.1 |
| GOh1021 | 120 | N268W | 3.3 |
| GOh1015 | 121 | F217V | 2.1 |
| GOh1017 | 128 | F217Q | 132.2 |
| GOh1036 | 128 | F217A | 101.9 |
| GOh1039 | 128 | F217G | 87.9 |
| GOh1042 | 128 | F217M | 73.0 |
| GOh1015 | 128 | F217V | 68.7 |
| GOh1018 | 128 | F217N | 67.7 |
| GOh1043 | 128 | F217R | 40.8 |
| GOh1021 | 128 | N268W | 39.8 |
| GOh1030 | 128 | F217I | 22.9 |
| GOh1016 | 128 | F217P | 20.1 |
| GOh1031 | 128 | F217T | 15.0 |
| GOh1020 | 128 | F217S | 13.6 |
| GOh1034 | 128 | F217E | 5.6 |
| GOh1008 | 128 | S314C | 4.9 |
| GOh1009 | 128 | S314K | 4.3 |
| GOh1010 | 128 | S314L | 3.5 |
| GOh1006 | 128 | M353F | 3.1 |
| GOh1023 | 128 | N268F | 3.1 |
| GOh1025 | 128 | E218P | 3.1 |
| GOh1013 | 128 | L537G | 2.5 |
| GOh1024 | 128 | N268K | 1.9 |
| GOh1012 | 128 | S314T | 1.5 |
| GOh1052 | 166 | F217A, N268W | 3643.3 |
| GOh1048 | 166 | F217V, E218P | 452.9 |
| GOh1051 | 166 | E218P, L537G | 192.2 |
| GOh1045 | 166 | F217P, E218P | 188.9 |
| GOh1046 | 166 | F217P, L537G | 85.4 |
| GOh1049 | 166 | F217V, L537G | 58.3 |
| GOh1055 | 166 | F217G, N268W | 47.4 |

39 purified enzymes show improved activity profile benchmarked against M3-5 backbone (Table 2). The experimental conditions used were: Purified protein (10 μL, 400 μg/mL), 5 μL HRP (1 mg/mL), 2.5 mM ABTS, 5% DMSO v/v, 1 mM substrate, 25° C., topped up to 200 μL with 100 mM sodium phosphate (pH 7.0) buffer, 24 hours.

TABLE 3
The colorimetric activity of purified galactose oxidase
enzymes activity profile benchmarked against GOh1052
(F217A, N268W from M3-5) backbone for substrate 152.

| Mutant | Mutations | Activity Fold |
|---|---|---|
| No. | (on GOh1052) | Change |
| GOh1052 | backbone | 1.0 |
| GOh2015 | A197C | 22.2 |
| GOh2010 | Y352F | 18.2 |
| GOh2072 | S114R | 13.3 |
| GOh2013 | Y352T | 9.5 |
| GOh2026 | K389I | 8.2 |
| GOh2011 | G294A | 8.1 |
| GOh2016 | R240G | 6.2 |
| GOh2021 | T202I | 5.9 |
| GOh2025 | A346L | 4.1 |
| GOh2002 | V193T | 1.8 |
| GOh2019 | T202D | 1.8 |
| GOh2020 | T202Q | 1.8 |
| GOh2018 | K348S | 1.5 |
| GOh2022 | T202K | 1.4 |

14 purified enzymes show improved activity profile benchmarked against GOh1052 (F217A, N268W from M3-5) backbone for substrate 152 (Table 3). The experimental conditions used were: Purified protein (10 μL, 400 μg/mL), 5 μL HRP (1 mg/mL), 2.5 mM ABTS, 5% DMSO v/v, 1 mM substrate 152, 25° C., topped up to 200 μL with 100 mM sodium phosphate (pH 7.0) buffer, 24 hours.

TABLE 4
The High Performance Liquid Chromatography (HPLC)
Yield Activity of purified galactose oxidase enzymes
activity profile benchmarked against GOh1052 (F217A,
N268W from M3-5) backbone for substrate 152.

| Mutant | Mutations | Activity Fold |
|---|---|---|
| No. | (on GOh1052) | Change |
| GOh1052 | backbone | 1.00 |
| GOh2082 | E218F | 2.68 |
| GOh2081 | A195V | 2.04 |
| GOh2044 | A24H | 2.00 |
| GOh2086 | I28F | 1.84 |
| GOh2012 | A195T | 1.80 |
| GOh2027 | Y352W | 1.76 |
| GOh2045 | I28R | 1.76 |
| GOh2085 | A26R | 1.76 |
| GOh2070 | D239E | 1.68 |
| GOh2084 | I224W | 1.64 |
| GOh2087 | E218Y | 1.60 |
| GOh2083 | L226Q | 1.56 |
| GOh2091 | A24N | 1.52 |
| GOh2023 | P194C | 1.36 |
| GOh2092 | A26H | 1.36 |
| GOh2039 | E218K | 1.28 |
| GOh2090 | F546M | 1.28 |
| GOh2001 | A195S | 1.24 |
| GOh2080 | S173K | 1.20 |
| GOh2014 | N341W | 1.16 |
| GOh2076 | S173R | 1.16 |
| GOh2079 | Q662D | 1.16 |
| GOh2008 | N259T | 1.12 |
| GOh2043 | I284Y | 1.08 |
| GOh2046 | I28M | 1.08 |
| GOh2047 | S576D | 1.08 |
| GOh2017 | A198I | 1.04 |
| GOh2048 | I284S | 1.04 |
| GOh2055 | A197G | 1.04 |
| GOh2071 | T117D | 1.04 |
| GOh2075 | Q662P | 1.04 |
| GOh2078 | S172E | 1.04 |
| GOh2089 | D239Q | 1.04 |

33 purified enzymes show improved activity profile benchmarked against GOh1052 (F217A, N268W from M3-5) backbone for substrate 152 (Table 4). The experimental conditions used were: purified protein (20 μL, 400 μg/mL), 5 μL HRP (1 mg/mL), 5% DMSO v/v, 1 mM substrate 152, 25° C., 220 rpm, topped up to 200 μL with 100 mM sodium phosphate (pH 7.0) buffer (in 2 mL HPLC vials), 6 hours.

TABLE 5
The 40° C. Activity of purified galactose oxidase
enzymes activity profile benchmarked against GOh1052
(F217A, N268W from M3-5) backbone for substrate 152.

| Mutant | Mutations | HPLC Yield |
|---|---|---|
| No. | (on GOh1052) | of Ketone |
| GOh1052 | backbone | 33% |
| GOh3006 | I28R, K389I | 72% |
| GOh3008 | I28R, T153R, A198I, N341W | 64% |
| GOh3038 | I28R, A195T, N259T, N341W, K389I | 64% |
| GOh3013 | I28R, A195T, N259T, K389I | 63% |
| GOh3026 | I28R, A195T, N259T, T447A | 63% |
| GOh3023 | I28R, A195T, N341W, K389I | 61% |
| GOh2045 | I28R | 61% |
| GOh2044 | A24H | 59% |
| GOh2070 | D239E | 57% |
| GOh3032 | I28R, A195S, N259T, N341W | 56% |
| GOh3015 | I28R, N259T, N341W | 52% |
| GOh3018 | I28R, A195S, K389I | 52% |
| GOh2082 | E218F | 52% |
| GOh2083 | L226Q | 52% |
| GOh3003 | A198I, N259T | 51% |
| GOh2014 | N341W | 50% |
| GOh2017 | A198I | 50% |
| GOh2091 | A24N | 49% |
| GOh2086 | I28F | 47% |
| GOh2087 | E218Y | 47% |
| GOh2084 | I224W | 46% |
| GOh2085 | A26R | 44% |
| GOh2023 | P194C | 43% |
| GOh2119 | A401D | 43% |
| GOh2027 | Y352W | 41% |
| GOh2055 | A197G | 41% |
| GOh2089 | D239Q | 41% |
| GOh2092 | A26H | 41% |
| GOh2117 | T175K | 41% |
| GOh2081 | A195V | 40% |
| GOh2039 | E218K | 39% |
| GOh2114 | T175E | 39% |
| GOh2115 | T175H | 39% |
| GOh2008 | N259T | 38% |
| GOh2043 | I284Y | 36% |
| GOh2046 | I28M | 36% |
| GOh2098 | Q45E | 36% |
| GOh2116 | T175I | 36% |
| GOh2071 | T117D | 35% |
| GOh2090 | F546M | 35% |
| GOh2111 | W104N | 35% |
| GOh3002 | N341W, Y352T | 34% |
| GOh2048 | I284S | 34% |
| GOh2056 | T265S | 34% |
| GOh2076 | S173R | 34% |

45 purified enzymes show improved heated activity profile benchmarked against GOh1052 (F217A, N268W from M3-5) backbone for substrate 152 (Table 5). The experimental conditions used were: purified protein (20 μL, 400 μg/mL), 5 μL HRP (1 mg/mL), 5% DMSO v/v, 1 mM substrate 152, 40° C., 220 rpm, topped up to 200 μL with 100 mM sodium phosphate (pH 7.0) buffer (in 2 mL HPLC vials), 6 hours.

TABLE 6
The activity, stability, solubility, expression, and enantioselectivity of modified galactose oxidases

| | Mutations | | | | | | |
|---|---|---|---|---|---|---|---|
| | on top of | Activity | Activity | Thermal | | | Relaxed |
| Name | M3-5 | (HPLC) | (Colorimetric) | Stability | Solubility | Expression | Enantioselectivity |
| GOh1006 | M353F | | Y+ | Y | Y | Y | |
| GOh1008 | S314C | | Y+ | | Y | Y | |
| GOh1013 | L537G | Y | Y+ | | Y | Y | |
| GOh1015 | F217V | Y | Y+ | | | | Y |
| GOh1016 | F217P | Y | Y+ | | | | Y |
| GOh1017 | F217Q | Y | Y+ | | | Y | |
| GOh1018 | F217N | Y | Y+ | | | Y | |
| GOh1020 | F217S | Y | Y+ | Y | | Y | Y |
| GOh1021 | N268W | Y | Y+ | | Y | | |
| GOh1025 | E218P | Y | Y+ | | | Y | |
| GOh1030 | F217I | Y | | | | | Y |
| GOh1031 | F217T | Y | | | Y | | Y |
| GOh1032 | F217L | | | | Y | | Y |
| GOh1034 | F217E | | Y+ | | Y | | Y |
| GOh1035 | F217Y | | | | Y | Y | |
| GOh1036 | F217A | | Y+ | Y | Y | | Y |
| GOh1037 | F217C | | | | | | Y |
| GOh1038 | F217D | | | | Y | | Y |
| GOh1039 | F217G | | Y+ | | | Y | Y |
| GOh1040 | F217H | | | | | Y | Y |
| GOh1041 | F217K | | | | Y | | Y |
| GOh1042 | F217M | | Y+ | | | | Y |
| GOh1043 | F217R | | Y+ | | | | Y |
| GOh1045 | F217P, E218P | | Y+ | | | | |
| GOh1046 | F217P, L537G | | Y+ | | | | |
| GOh1047 | F217P, E218P, | | Y+ | | | | |
| | L537G | | | | | | |
| GOh1048 | F217V, E218P | | Y+ | | | | |
| GOh1049 | F217V, L537G | | Y+ | | | | |
| GOh1050 | F217V, E218P, | | Y+ | | | | |
| | L537G | | | | | | |
| GOh1051 | E218P, L537G | | Y+ | | | | |
| GOh1052 | F217A, N268W | | Y+ | | | | |
| GOh1053 | F217A, N268W, | | Y+ | | | | |
| | M353F | | | | | | |
| GOh1054 | F217A, N268W, | | Y+ | | | | |
| | S314C | | | | | | |
| GOh1055 | F217G, N268W | | Y+ | | | | |
| GOh1056 | F217G, N268W, | | Y+ | | | | |
| | M353F | | | | | | |
| GOh2001 | F217A, N268W, | Y | Y+ | | Y | | Y |
| | A195S | | | | | | |
| GOh2002 | F217A, N268W, | | Y+ | | | | |
| | V193T | | | | | | |
| GOh2008 | F217A, N268W, | | Y+ | Y | | | |
| | N259T | | | | | | |
| GOh2010 | F217A, N268W, | | Y+ | | | | |
| | Y325F | | | | | | |
| GOh2011 | F217A, N268W, | | Y+ | | | | |
| | G294A | | | | | | |
| GOh2012 | F217A, N268W, | Y | Y+ | | Y | | |
| | A195T | | | | | | |
| GOh2013 | F217A, N268W, | | Y+ | | | | |
| | Y352T | | | | | | |
| GOh2014 | F217A, N268W, | | Y+ | Y | | Y | Y |
| | N341W | | | | | | |
| GOh2015 | F217A, N268W, | | Y+ | | | | |
| | A197C | | | | | | |
| GOh2016 | F217A, N268W, | | Y+ | | | | |
| | R240G | | | | | | |
| GOh2017 | F217A, N268W, | | Y+ | Y | | | |
| | A198I | | | | | | |
| GOh2018 | F217A, N268W, | | Y+ | | | | |
| | K348S | | | | | | |
| GOh2019 | F217A, N268W, | | Y+ | | | | Y |
| | T202D | | | | | | |
| GOh2020 | F217A, N268W, | | Y+ | | | Y | |
| | T202Q | | | | | | |
| GOh2021 | F217A, N268W, | | Y+ | Y | | Y | |
| | T202I | | | | | | |
| GOh2022 | F217A, N268W, | | Y+ | | | Y | |
| | T202K | | | | | | |
| GOh2023 | F217A, N268W, | Y | Y+ | Y | | Y | |
| | P194C | | | | | | |
| GOh2025 | F217A, N268W, | | Y+ | | | Y | Y |
| | A346L | | | | | | |
| GOh2026 | F217A, N268W, | | Y+ | | Y | Y | |
| | K389I | | | | | | |
| GOh2027 | F217A, N268W, | Y | Y+ | | | Y | Y |
| | Y352W | | | | | | |
| GOh2028 | F217A, N268W, | | Y+ | | | Y | |
| | C538K | | | | | | |
| GOh2029 | F217A, N268W, | | Y+ | | Y | Y | |
| | C538R | | | | | | |
| GOh2030 | F217A, N268W, | | | | Y | | |
| | F250E | | | | | | |
| GOh2031 | F217A, N268W, | | | | Y | | |
| | F250Y | | | | | | |
| GOh2032 | F217A, N268W, | | Y+ | | Y | | |
| | N356K | | | | | | |
| GOh2033 | F217A, N268W, | | Y+ | | Y | Y | |
| | N356R | | | | | | |
| GOh2034 | F217A, N268W, | | Y+ | | Y | Y | |
| | N356W | | | | | | |
| GOh2037 | F217A, N268W, | | Y+ | | | | |
| | F487R | | | | | | |
| GOh2038 | F217A, N268W, | | | Y | Y | Y | |
| | T429W | | | | | | |
| GOh2039 | F217A, N268W, | Y | Y+ | | | | |
| | E218K | | | | | | |
| GOh2041 | F217A, N268W, | | | Y | Y | Y | |
| | F313R | | | | | | |
| GOh2043 | F217A, N268W, | | Y+ | Y | | | |
| | I284Y | | | | | | |
| GOh2044 | F217A, N268W, | Y | Y+ | Y | | | |
| | A24H | | | | | | |
| GOh2045 | F217A, N268W, | Y | Y+ | Y | Y | | |
| | I28R | | | | | | |
| GOh2046 | F217A, N268W, | | Y+ | Y | Y | | |
| | I28M | | | | | | |
| GOh2047 | F217A, N268W, | | | | Y | | |
| | S576D | | | | | | |
| GOh2048 | F217A, N268W, | | Y+ | Y | Y | | |
| | I284S | | | | | | |
| GOh2050 | F217A, N268W, | | | Y | Y | | |
| | F65P | | | | | | |
| GOh2053 | F217A, N268W, | | | Y | | | |
| | T153R | | | | | | |
| GOh2055 | F217A, N268W, | | | Y | Y | | |
| | A197G | | | | | | |
| GOh2056 | F217A, N268W, | | | Y | Y | | |
| | T265S | | | | | | |
| GOh2057 | F217A, N268W, | | | | Y | | |
| | S220T | | | | | | |
| GOh2058 | F217A, N268W, | | | | Y | | |
| | N214S | | | | | | |
| GOh2061 | F217A, N268W, | | | | Y | | |
| | I485A | | | | | | |
| GOh2064 | F217A, N268W, | | | Y | Y | | |
| | N214R | | | | | | |
| GOh2066 | F217A, N268W, | | | | Y | Y | |
| | D269N | | | | | | |
| GOh2067 | F217A, N268W, | | | | Y | Y | |
| | I485R | | | | | | |
| GOh2068 | F217A, N268W, | | | | Y | | |
| | I485K | | | | | | |
| GOh2070 | F217A, N268W, | Y | Y+ | Y | | Y | |
| | D239E | | | | | | |
| GOh2071 | F217A, N268W, | | Y+ | | | | |
| | T117D | | | | | | |
| GOh2072 | F217A, N268W, | | Y+ | | Y | Y | |
| | S114R | | | | | | |
| GOh2073 | F217A, N268W, | | | | Y | Y | |
| | S559P | | | | | | |
| GOh2074 | F217A, N268W, | | | | Y | | |
| | S114N | | | | | | |
| GOh2075 | F217A, N268W, | | Y+ | | Y | | |
| | Q662P | | | | | | |
| GOh2076 | F217A, N268W, | | Y+ | | | | |
| | S173R | | | | | | |
| GOh2077 | F217A, N268W, | | | | Y | | |
| | D239P | | | | | | |
| GOh2078 | F217A, N268W, | | | | Y | Y | |
| | S172E | | | | | | |
| GOh2079 | F217A, N268W, | | Y+ | | Y | Y | |
| | Q662D | | | | | | |
| GOh2080 | F217A, N268W, | Y | Y+ | | Y | | |
| | S173K | | | | | | |
| GOh2081 | F217A, N268W, | Y | Y+ | | Y | Y | |
| | A195V | | | | | | |
| GOh2082 | F217A, N268W, | Y | Y+ | Y | Y | Y | |
| | E218F | | | | | | |
| GOh2083 | F217A, N268W, | Y | | Y | Y | Y | |
| | L226Q | | | | | | |
| GOh2084 | F217A, N268W, | Y | | Y | Y | | |
| | I224W | | | | | | |
| GOh2085 | F217A, N268W, | Y | | Y | | | |
| | A26R | | | | | | |
| GOh2086 | F217A, N268W, | Y | Y+ | Y | | | |
| | I28F | | | | | | |
| GOh2087 | F217A, N268W, | Y | | Y | Y | | |
| | E218Y | | | | | | |
| GOh2088 | F217A, N268W, | | | | Y | | |
| | L226V | | | | | | |
| GOh2089 | F217A, N268W, | | | Y | Y | | |
| | D239Q | | | | | | |
| GOh2090 | F217A, N268W, | Y | | | Y | | |
| | F546M | | | | | | |
| GOh2091 | F217A, N268W, | Y | Y+ | Y | Y | | |
| | A24N | | | | | | |
| GOh2092 | F217A, N268W, | Y | | Y | | | |
| | A26H | | | | | | |
| GOh2093 | F217A, N268W, | | | Y | | Y | |
| | Y599M | | | | | | |
| GOh2094 | F217A, N268W, | | | Y | Y | Y | |
| | G157N | | | | | | |
| GOh2097 | F217A, N268W, | | | Y | Y | | |
| | Q45D | | | | | | |
| GOh2098 | F217A, N268W, | | | Y | Y | Y | |
| | Q45E | | | | | | |
| GOh2099 | F217A, N268W, | | | | | Y | |
| | Q45N | | | | | | |
| GOh2100 | F217A, N268W, | | | Y | | | |
| | E49K | | | | | | |
| GOh2101 | F217A, N268W, | | | | Y | Y | |
| | N69D | | | | | | |
| GOh2102 | F217A, N268W, | | | | Y | Y | |
| | N69E | | | | | | |
| GOh2103 | F217A, N268W, | | | Y | | | |
| | N69F | | | | | | |
| GOh2104 | F217A, N268W, | | | Y | | | |
| | N69H | | | | | | |
| GOh2105 | F217A, N268W, | | | Y | | Y | |
| | N69L | | | | | | |
| GOh2106 | F217A, N268W, | | | Y | | | |
| | N69M | | | | | | |
| GOh2107 | F217A, N268W, | | | Y | | | |
| | N69T | | | | | | |
| GOh2108 | F217A, N268W, | | | Y | | | |
| | N69W | | | | | | |
| GOh2109 | F217A, N268W, | | | Y | | Y | |
| | N69Y | | | | | | |
| GOh2110 | F217A, N268W, | Y | | | | Y | |
| | W104G | | | | | | |
| GOh2111 | F217A, N268W, | Y | | Y | | Y | |
| | W104N | | | | | | |
| GOh2114 | F217A, N268W, | Y | | Y | | Y | |
| | T175E | | | | | | |
| GOh2115 | F217A, N268W, | Y | | Y | | Y | |
| | T175H | | | | | | |
| GOh2116 | F217A, N268W, | | | Y | | Y | |
| | T175I | | | | | | |
| GOh2117 | F217A, N268W, | Y | | Y | Y | Y | |
| | T175K | | | | | | |
| GOh2119 | F217A, N268W, | Y | | | Y | Y | |
| | A401D | | | | | | |
| GOh3001 | F217A, N268W, | Y | Y+ | | Y | | |
| | A195T, Y352W | | | | | | |
| GOh3002 | F217A, N268W, | Y | | Y | | Y | |
| | N341W, Y352T | | | | | | |
| GOh3003 | F217A, N268W, | | | Y | | | |
| | A198I, N259T | | | | | | |
| GOh3004 | F217A, N268W, | | | | | Y | |
| | A195S, N341W | | | | | | |
| GOh3005 | F217A, N268W, | Y | | | Y | Y | |
| | T153K, A195S | | | | | | |
| GOh3006 | F217A, N268W, | Y | Y+ | Y | | | |
| | I28R, K389I | | | | | | |
| GOh3007 | F217A, N268W, | Y | | | | | |
| | T153R, A195T | | | | | | |
| GOh3008 | F217A, N268W, | Y | | Y | | | |
| | I28R, T153R, | | | | | | |
| | A198I, N341W | | | | | | |
| GOh3009 | F217A, N268W, | Y | | | | Y | |
| | A195T, N259T, | | | | | | |
| | N341W | | | | | | |
| GOh3013 | F217A, N268W, | Y | Y+ | Y | Y | | |
| | I28R, A195T, | | | | | | |
| | N259T, K389I | | | | | | |
| GOh3015 | F217A, N268W, | Y | | Y | Y | Y | |
| | I28R, N259T, | | | | | | |
| | N341W | | | | | | |
| GOh3018 | F217A, N268W, | Y | | Y | Y | | |
| | I28R, A195S, | | | | | | |
| | K389I | | | | | | |
| GOh3019 | F217A, N268W, | Y | | | Y | Y | |
| | A195T, N341W | | | | | | |
| GOh3023 | F217A, N268W, | Y | | | Y | | |
| | I28R, A195T, | | | | | | |
| | N341W, K389I | | | | | | |
| GOh3026 | F217A, N268W, | Y | | Y | Y | | |
| | I28R, A195T, | | | | | | |
| | N259T, T447A | | | | | | |
| GOh3032 | F217A, N268W, | Y | | Y | Y | Y | |
| | I28R, A195S, | | | | | | |
| | N259T, N341W | | | | | | |
| GOh3038 | F217A, N268W, | Y | Y+ | Y | | | |
| | I28R, A195T, | | | | | | |
| | N259T, N341W, | | | | | | |
| | K389I | | | | | | |
| GOh3049 | F217A, N268W, | | | Y | | | |
| | T153R, N341W | | | | | | |

For “Activity (HPLC)”:

Interpretation is as follows:
(a) if a “Y” is obtained for a GOh1XXX modified galactose oxidase tested, then the activity of said modified galactose oxidase is at least 10-fold better than the M3-5 variant for substrate 128 (alpha-tetralol); and
(b) if a “Y” is obtained for a GOh2XXX or GOh3XXX modified galactose oxidase tested, then the activity of said modified galactose oxidase is at least 1.2-fold better than GOh1052 for substrate 152 (1-phenyl-1-butanol).

For GOhXXXX variant activity—the minimum fold change for said variant to be considered as having better activity is 1.01-fold better than M3-5 for 1 or more substrates in the 64-substrate panel (indicated in FIG. 1 of the present disclosure).

For “Activity (Colorimetric)”:

Interpretation is as follows:
(a) if a “Y+” is obtained for any of modified galactose oxidase tested, then said modified galactose oxidase is better than the M3-5 variant for some substrate in the colorimetric substrate panel.

For “Stability” (thermal stability), “Solubility” and “Expression” (protein expression):

Interpretation is as follows:
(a) if a “Y” is obtained for a GOh1XXX modified galactose oxidase tested, then the properties of said modified galactose oxidase is better than the M3-5 variant by 1.01-fold; and (b) if a “Y” is obtained for a GOh2XXX or GOh3XXX modified galactose oxidase tested, then the properties of said modified galactose oxidase is better than Goh1052 by 1.01-fold.

For the measure of “Relaxed Enantioselectivity”

Interpretation is as follows:
(a) if a “Y” is obtained for any of the modified galactose oxidase tested, then the enantioselectivity of said modified galactose oxidase is reduced by 1.01-fold compared to that of the M3-5 variant baseline.

For ease of comparison, interpretation of reduced enantioselectivity is conveyed as absolute units as a ratio in conversion of (R)- and (S)-enantiomers of the substrate. Higher R/S ratios indicate higher enantioselectivity for (R)-enantiomers while R/S ratios closer to 1 indicate reduced enantioselectivity, with 1 R/S ratio being no enantioselectivity.

For GOh1XXX enantioselectivity—minimum reduction of 0.8 R/S ratio from M3-5 experimental value of 20.6 R/S ratio (for substrate 128—alpha-tetralol)

For GOh2XXX and GOh3XXX enantioselectivity—minimum reduction of 0.22 R/S ratio from GOh1052 experimental value of 1.67 R/S ratio (for substrate 152-1-phenyl-1-butanol).

### Results

### Identification of Galactose Oxidase Mutants

High throughput screening of 6,692 galactose oxidase variants from 352 amino acid positions against substrate 152 have revealed that 31 galactose oxidase variants (0.46%) show improved enzymatic activity (>1.2×baseline, Table 7). The oxidation reaction of selected examples of bulky benzylic, unactivated, and heterocyclic aliphatic secondary alcohol substrates to ketones by galactose oxidase mutants are shown in the chemical equation below:

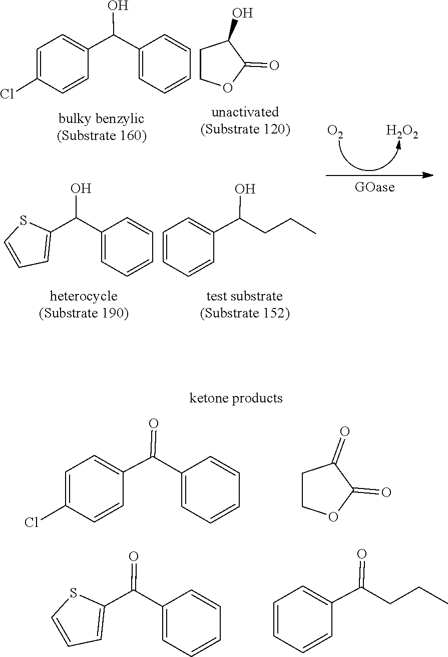

TABLE 7
Table showing the Activity Hits from High Throughput
Screening of 6,692 galactose oxidase variants

| | Activity vs |
|---|---|
| Variant | GOh1052 |
| E218F | 2.13 |
| Y599M | 1.84 |
| L226Q | 1.66 |
| A24H | 1.51 |
| A195V | 1.49 |
| A26R | 1.35 |
| I28A | 1.33 |
| L226R | 1.33 |
| I224W | 1.32 |
| I28M | 1.31 |
| D239Q | 1.31 |
| K508P | 1.31 |
| A401D | 1.30 |
| D239E | 1.30 |
| I28P | 1.29 |
| E218Y | 1.29 |
| A26F | 1.28 |
| A24N | 1.28 |
| I28D | 1.28 |
| T542G | 1.28 |
| L226V | 1.27 |
| I28R | 1.26 |
| I28E | 1.26 |
| G157N | 1.26 |
| A26H | 1.25 |
| E218W | 1.25 |
| T616V | 1.23 |
| I28F | 1.23 |
| A24G | 1.22 |
| I28H | 1.21 |
| A26D | 1.20 |

31 Activity Hits were obtained from High Throughput Screening of 6,692 galactose oxidase variants. For the screening, GOh1052 was used as the backbone with 10 mutations (S33P, M93V, F217A, G218E, N268W, W313F, R353M, Q429T, V517A, N558D) from wild-type galactose oxidase from Fusarium graminearum. Screening conditions are described in paragraph [0109] and the Activity calculations are described in paragraph [0128] above.

TABLE 8
Selected first generation galactose oxidase variant examples
based on M3-5 backbone with improved activity for chosen
bulky benzylic and unactivated alcohol substrates.

| | | Activity | | |
|---|---|---|---|---|
| | | (bulky | Activity | |
| | | benzylic - | (unactivated - | |
| | | substrate | substrate | |
| Name | Mutations | 160) | 120) | Comment |
| M3-5 | backbone | 1.54 | 135 | Baseline |
| GOh1008 | S314C | 2.04 | 264 | unactivated activity |
| GOh1021 | N268W | 5.47 | 446 | unactivated activity |
| GOh1036 | F217A | 6921 | 4.50 | bulky benzylic |
| | | | | activity |
| GOh1052 | F217A, | 5884 | 2.87 | generalist activity |
| | N268W | | | |

As described in paragraph [0110] above, Activity=initial rate*max absorbance*100. Max absorbance=indirect measure of ketone yield based on 405 nm absorbance of a colorimetric probe (ABTS) measuring H₂O₂.

TABLE 9
Selected second generation variant examples based on GOh1052
(F217A, N268W from M3-5) backbone with improved properties. Up to
10⁴-fold activity improvement from baseline M3-5 variant.

| | | Activity | Activity | | | |
|---|---|---|---|---|---|---|
| | | (bulky | (hetero- | R/S | Residual | |
| | | benzylic- | cycle - | Ratio^{a} - | Activity^{b}- | |
| | | substrate | substrate | substrate | substrate | |
| Name | Mutations | 160) | 190) | 152 | 152 | Comment |
| GOh1052 | backbone | 5884 | 4862 | 1.67 | 56% | baseline |
| GOh2001 | A195S | 5996 | 2125 | 1.05 | 39% | activity, relaxed |
| | | | | | | enantioselectivity |
| GOh2008 | N259T | 6529 | 5394 | 2.00 | 84% | activity |
| GOh2012 | A195T | 6393 | 2874 | 1.73 | 3% | activity |
| GOh2017 | A198I | 5662 | 1034 | — | 79% | thermostable |
| GOh2021 | T202I | 2376 | 296 | — | 74% | thermostable |
| GOh2027 | Y352W | 2116 | 1885 | 1.45 | 38% | ortho-substrate activity |
| GOh2044 | A24H | 8582 | 36573 | — | 74% | activity |
| GOh2045 | I28R | 6469 | 9346 | — | 79% | activity & thermostable |
| GOh2070 | D239E | 11412 | 8105 | — | 78% | activity & thermostable |
| GOh2091 | A24N | — | — | — | 82% | activity & thermostable |

As described in paragraph [0110] above, Activity = initial rate * max absorbance * 100. Max absorbance = indirect measure of ketone yield based on 405 nm absorbance of a colorimetric probe (ABTS) measuring H₂O₂.
^{a}R/S ratio [(R-isomer substrate 152 converted)/(S-isomer substrate 152 converted)] starting from a 50/50 mixture of each.
^{b}Residual activity = (substrate 152 ketone yield using enzyme heated at 50° C. for 30 min)/(substrate 152 ketone yield using fresh enzyme).

As described in paragraph [0110] above, Activity=initial rate*max absorbance*100. Max absorbance=indirect measure of ketone yield based on 405 nm absorbance of a colorimetric probe (ABTS) measuring H₂O₂. ^{a}R/S ratio=[(R-isomer substrate 152 converted)/(S-isomer substrate 152 converted)] starting from a 50/50 mixture of each. ^{b}Residual activity=(substrate 152 ketone yield using enzyme heated at 50° C. for 30 min)/(substrate 152 ketone yield using fresh enzyme).

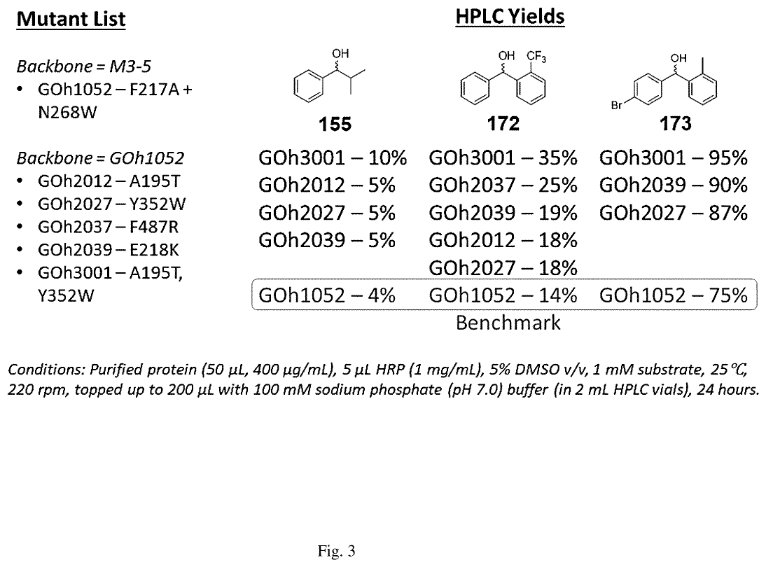

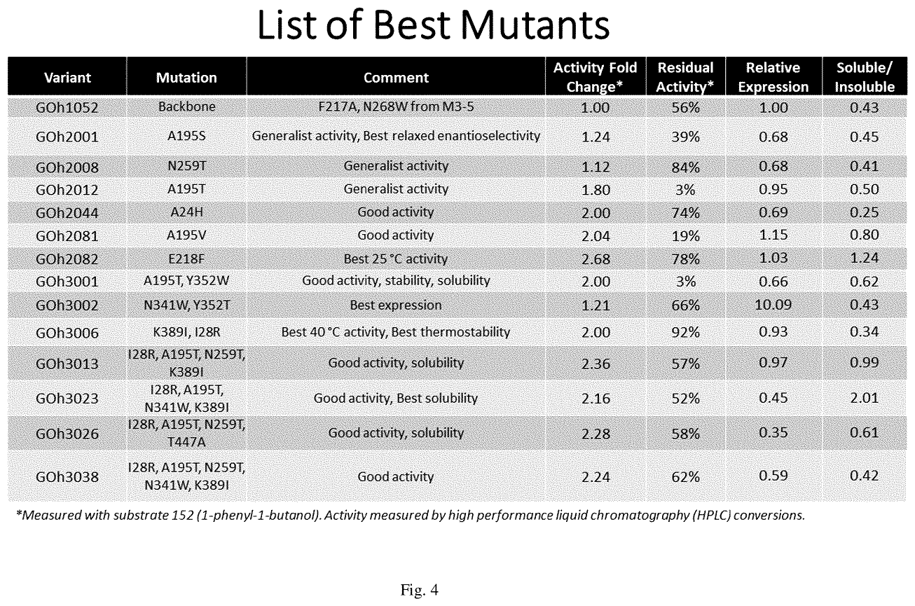

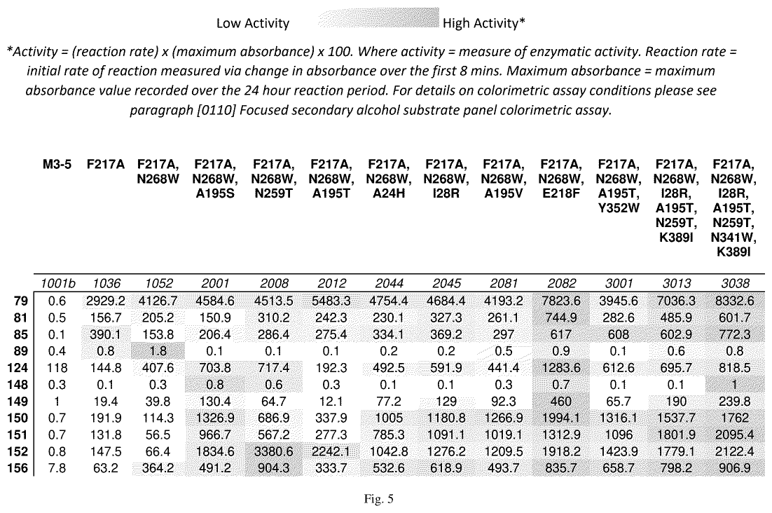

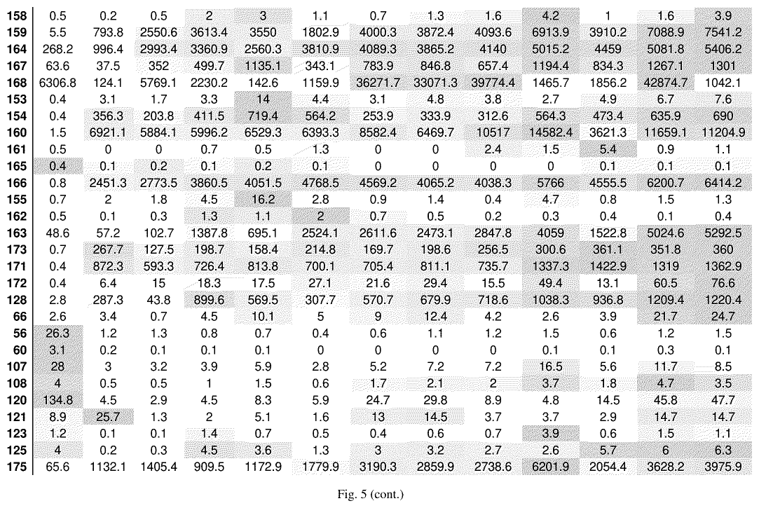

Selected examples of additional combinatorial third generation variants (incorporating 4 or more synergistic mutations to the M3-5 variant) including galactose oxidase variant GOh3001 (A195T, F217A, N268W, Y352W) showed improved generalist activity and improved acceptance of ortho-substituted substrates (FIG. 3), and galactose oxidase variant GOh3006 (I28R, F217A, N268W, K389I) showed synergistic mutations that combine desirable traits of high activity and thermostability (FIG. 4).

### Synthesis of Active Pharmaceutical Ingredient (API) Molecules Using Galactose Oxidase Variants

Identified beneficial enzyme mutations were applied to synthesize API molecules. The chemical equation below shows two examples of the biocatalytic synthesis of APIs by galactose oxidase variant GOh1052.

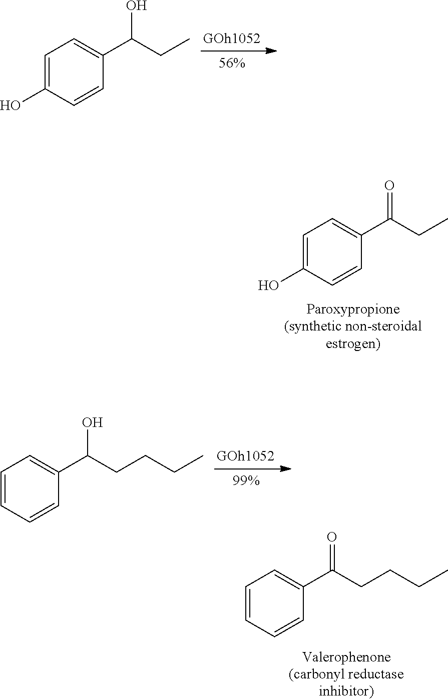

### Analysis of the Enzyme Generalist Activity of 12 Galactose Oxidase Mutants

To determine the enzyme generalist activity of galactose oxidase mutants, purified galactose oxidase enzyme mutants were screened against a panel of 60 secondary alcohols. The screening conditions were:
10 μL purified enzyme (400 μg/mL)
5 μL horseradish peroxidase (1 mg/mL)
1 mM secondary alcohol substrate
2.5 mM ABTS (colorimetric indicator)
5% dimethylsulfoxide v/v in 100 mM sodium phosphate buffer (pH=7)
200 μL total reaction volume

Table 10 below lists 12 galactose oxidase mutants with good enzyme generalist activity compared to the M3-5 variant across the 60-substrate panel. The 60 secondary alcohol substrate panel is shown in FIG. 1. The activity fold improvements have been rounded to the nearest 100.

TABLE 10
Table of 12 galactose oxidase mutants with good generalist
activity across the 60-substrate panel.

| | | Averaged |
|---|---|---|
| | | Activity |
| | | Fold |
| | | vs M3-5 |
| | | (across 60 |
| Mutant | Mutations | Substrates) |
| GOh1001b (M3-5) | Backbone | 1 |
| GOh1036 | F217A | 900 |
| GOh1052 | F217A, N268W | 600 |
| GOh2001 | F217A, N268W, A195S | 600 |
| GOh2008 | F217A, N268W, N259T | 800 |
| GOh2012 | F217A, N268W, A195T | 900 |
| GOh2044 | F217A, N268W, A24H | 1500 |
| GOh2045 | F217A, N268W, I28R | 1200 |
| GOh2081 | F217A, N268W, A195V | 1100 |
| GOh2082 | F217A, N268W, E218F | 2000 |
| GOh3001 | F217A, N268W, A195T, Y352W | 1000 |
| GOh3013 | F217A, N268W, I28R, A195T, | 2000 |
| | N259T, K389I | |
| GOh3038 | F217A, N268W, I28R, A195T, | 2100 |
| | N259T, N341W, K389I | |

The activity values for 13 galactose oxidase mutants against 60 substrates are shown in FIG. 5 (colour comparisons are done horizontally per substrate). FIG. 5 shows that the 13 galactose oxidase mutants had generally higher activity values compared to the M3-5 variant. The “Averaged Activity Fold” was obtained by sequentially: (1) taking the colorimetric activity fold change of each substrate by the selected galactose oxidase variant (e.g., GOh1052) over benchmark M3-5; and (2) taking the average of all the fold changes of a given galactose oxidase variant (e.g. GOh1052) for all 60 substrates in FIG. 5).

### Analysis of the Thermostability of Galactose Oxidase Mutants

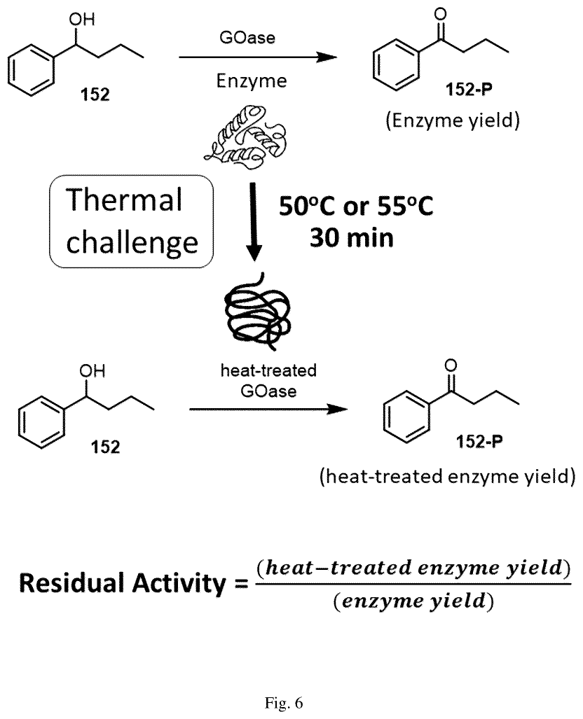

The thermostability profile of 55 purified galactose oxidase mutants were compared against the thermostability profile of GOh1052 (F217A, N268W from M3-5) backbone. As shown in FIG. 6, the purified galactose oxidase enzymes were heated at 50° C. or at 55° C. for 30 minutes, and the enzymatic activities of the resulting heat-treated enzymes were measured and compared with the GOh1052 control. Table 11 shows higher residual activity of the heat-treated galactose oxidase mutants compared to GOh1052 control.

TABLE 11
Table of 55 thermostable mutants compared to
GOh1052 (F217A, N268W from M3-5) backbone.

| | | Residual |
|---|---|---|
| | | Activity |
| Mutant | Mutations | (50° C. |
| No. | (on GOh1052) | 30 min) |
| GOh1052 | backbone | 56% |
| GOh3006 | I28R, K389I | 90% |
| GOh2008 | N259T | 84% |
| GOh2091 | A24N | 82% |
| GOh2104 | N69H | 82% |
| GOh2045 | I28R | 79% |
| GOh2105 | N69L | 79% |
| GOh2017 | A198I | 79% |
| GOh3015 | I28R, N259T, N341W | 78% |
| GOh2082 | E218F | 78% |
| GOh2070 | D239E | 78% |
| GOh2089 | D239Q | 76% |
| GOh3003 | A198I, N259T | 75% |
| GOh2041 | F313R | 75% |
| GOh2084 | I224W | 75% |
| GOh2021 | T202I | 74% |
| GOh2044 | A24H | 74% |
| GOh2106 | N69M | 74% |
| GOh3008 | I28R, T153R, A198I, N341W | 74% |
| GOh2086 | I28F | 73% |
| GOh2038 | T429W | 72% |
| GOh2093 | Y599M | 72% |
| GOh2097 | Q45D | 72% |
| GOh2100 | E49K | 72% |
| GOh2046 | I28M | 70% |
| GOh2087 | E218Y | 70% |
| GOh2109 | N69Y | 70% |
| GOh2114 | T175E | 70% |
| GOh3032 | I28R, A195S, N259T, | 69% |
| | N341W | |
| GOh3049 | T153R, N431W | 69% |
| GOh2083 | L226Q | 68% |
| GOh2014 | N341W | 68% |
| GOh2092 | A26H | 67% |
| GOh2085 | A26R | 67% |
| GOh3002 | N341W, Y352T | 66% |
| GOh2055 | A197G | 66% |
| GOh2115 | T175H | 66% |
| GOh2116 | T175I | 66% |
| GOh2043 | I284Y | 65% |
| GOh2103 | N69F | 65% |
| GOh2108 | N69W | 65% |
| GOh3018 | I28R, A195S, K389I | 65% |
| GOh2064 | N214R | 63% |
| GOh2048 | I284S | 62% |
| GOh2107 | N69T | 62% |
| GOh2117 | T175K | 62% |
| GOh3038 | I28R, A195T, N259T, | 62% |
| | N341W, K389I | |
| GOh2050 | F65P | 61% |
| GOh2098 | Q45E | 61% |
| GOh2023 | P194C | 60% |
| GOh2094 | G157N | 60% |
| GOh3026 | I28R, A195T, N259T, T447A | 58% |
| GOh2053 | T153R | 58% |
| GOh2056 | T265S | 57% |
| GOh2111 | W104N | 57% |
| GOh3013 | I28R, A195T, N259T, K389I | 57% |

TABLE 12
thermostable mutants compared to M3-5
control backbone for substrate 128

| | | Residual |
|---|---|---|
| | | Activity |
| Mutant | Mutations | (55° C. |
| No. | (on M3-5) | 30 min) |
| M3-5 | Backbone | 36% |
| GOh1036 | F217A | 58% |
| GOh1020 | F217S | 55% |
| GOh1006 | M353F | 55% |

### Analysis of the Protein Expression Profile of Galactose Oxidase Mutants

The protein expression profiles of purified galactose oxidase mutants were compared against the protein expression profile of GOh1052 (F217A, N268W from M3-5) backbone. Tables 13 and 14 show higher protein expression level of purified galactose oxidase mutants compared to the protein expression level of GOh1052 control and M3-5 control, respectively.

TABLE 13
Table showing improved protein expression profile
of 46 purified galactose oxidase mutants versus
GOh1052 (F217A, N268W from M3-5) backbone.

| Mutant | Mutations | Relative |
|---|---|---|
| No. | (on GOh1052) | Expression |
| GOh1052 | backbone | 1.00 |
| GOh3002 | N341W, Y352T | 10.09 |
| GOh2119 | A401D | 6.34 |
| GOh2117 | T175K | 4.29 |
| GOh2115 | T175H | 3.46 |
| GOh3004 | A195S, N341W | 3.36 |
| GOh2102 | N69E | 2.96 |
| GOh2116 | T175I | 2.58 |
| GOh2114 | T175E | 2.47 |
| GOh2101 | N69D | 2.06 |
| GOh2110 | W104G | 2.04 |
| GOh2041 | F313R | 2.00 |
| GOh2105 | N69L | 1.67 |
| GOh2021 | T202I | 1.64 |
| GOh2029 | C538R | 1.62 |
| GOh2109 | N69Y | 1.58 |
| GOh2022 | T202K | 1.52 |
| GOh2098 | Q45E | 1.47 |
| GOh2094 | G157N | 1.38 |
| GOh2099 | Q45N | 1.35 |
| GOh2038 | T429W | 1.34 |
| GOh2028 | C538K | 1.34 |
| GOh2020 | T202Q | 1.33 |
| GOh2066 | D269N | 1.32 |
| GOh3019 | A195T, N341W | 1.32 |
| GOh2025 | A346L | 1.31 |
| GOh2034 | N356W | 1.25 |
| GOh3005 | T153K, A195S | 1.22 |
| GOh3032 | I28R, A195S, N259T, N341W | 1.21 |
| GOh2111 | W104N | 1.19 |
| GOh2067 | I485R | 1.17 |
| GOh3015 | I28R, N259T, N341W | 1.17 |
| GOh2033 | N356R | 1.17 |
| GOh2081 | A195V | 1.15 |
| GOh2078 | S172E | 1.14 |
| GOh2026 | K389I | 1.14 |
| GOh2070 | D239E | 1.11 |
| GOh2072 | S114R | 1.11 |
| GOh2093 | Y599M | 1.08 |
| GOh2079 | Q662D | 1.05 |
| GOh2023 | P194C | 1.05 |
| GOh2073 | S559P | 1.03 |
| GOh2083 | L226Q | 1.03 |
| GOh2082 | E218F | 1.03 |
| GOh2027 | Y352W | 1.02 |
| GOh2014 | N341W | 1.01 |
| GOh3009 | A195T, N259T, N341W | 1.01 |

TABLE 14
Table showing improved protein expression profile of 10 purified
galactose oxidase mutants versus M3-5 control backbone.

| Mutant | Mutations | Relative |
|---|---|---|
| No. | (on M3-5) | Expression |
| M3-5 | Backbone | 1.00 |
| GOh1006 | M353F | 1.73 |
| GOh1008 | S314C | 1.51 |
| GOh1013 | L537G | 1.64 |
| GOh1017 | F217Q | 1.06 |
| GOh1018 | F217N | 1.16 |
| GOh1020 | F217S | 1.41 |
| GOh1025 | E218P | 1.36 |
| GOh1035 | F217Y | 1.10 |
| GOh1039 | F217G | 1.25 |
| GOh1040 | F217H | 1.19 |

### Analysis of the Protein Solubility of Galactose Oxidase Mutants

The protein solubilities of purified galactose oxidase mutants were compared against the protein solubility of GOh1052 (F217A, N268W from M3-5) backbone. Tables 15 and 16 show increased protein solubility of purified galactose oxidase mutants compared to the protein solubility of GOh1052 control, and M3-5 control, respectively.

TABLE 15
Table showing increased protein solubility of
58 purified galactose oxidase mutants versus
GOh1052 (F217A, N268W from M3-5) backbone.

| Mutant | Mutations | Soluble/Insoluble |
|---|---|---|
| No. | (on GOh1052) | Ratio |
| GOh1052 | backbone | 0.44 |
| GOh3023 | I28R, A195T, N341W, K389I | 2.01 |
| GOh2082 | E218F | 1.24 |
| GOh2056 | T265S | 1.12 |
| GOh2079 | Q662D | 1.03 |
| GOh3018 | I28R, A195S, K389I | 0.99 |
| GOh3013 | I28R, A195T, N259T, K389I | 0.99 |
| GOh2041 | F313R | 0.95 |
| GOh2084 | I224W | 0.84 |
| GOh2081 | A195V | 0.80 |
| GOh2068 | I485K | 0.79 |
| GOh2033 | N356R | 0.76 |
| GOh2119 | A401D | 0.74 |
| GOh2080 | S173K | 0.74 |
| GOh2047 | S576D | 0.73 |
| GOh2038 | T429W | 0.72 |
| GOh2075 | Q662P | 0.71 |
| GOh2067 | I485R | 0.71 |
| GOh3032 | I28R, A195S, N259T, N341W | 0.71 |
| GOh2083 | L226Q | 0.70 |
| GOh2073 | S559P | 0.68 |
| GOh2074 | S114N | 0.67 |
| GOh2048 | I284S | 0.66 |
| GOh2089 | D239Q | 0.65 |
| GOh2030 | F250E | 0.64 |
| GOh3019 | A195T, N341W | 0.64 |
| GOh2046 | I28M | 0.63 |
| GOh3001 | A195T, Y352W | 0.62 |
| GOh3015 | I28R, N259T, N341W | 0.61 |
| GOh2088 | L226V | 0.61 |
| GOh3026 | I28R, A195T, N259T, T447A | 0.61 |
| GOh3005 | T153K, A195S | 0.60 |
| GOh2050 | F65P | 0.60 |
| GOh2045 | I28R | 0.59 |
| GOh2091 | A24N | 0.58 |
| GOh2098 | Q45E | 0.58 |
| GOh2101 | N69D | 0.57 |
| GOh2064 | N214R | 0.57 |
| GOh2094 | G157N | 0.55 |
| GOh2032 | N356K | 0.53 |
| GOh2058 | N214S | 0.53 |
| GOh2087 | E218Y | 0.52 |
| GOh2061 | I485A | 0.52 |
| GOh2102 | N69E | 0.51 |
| GOh2078 | S172E | 0.51 |
| GOh2031 | F250Y | 0.50 |
| GOh2012 | A195T | 0.50 |
| GOh2117 | T175K | 0.49 |
| GOh2097 | Q45D | 0.48 |
| GOh2072 | S114R | 0.47 |
| GOh2055 | A197G | 0.47 |
| GOh2034 | N356W | 0.46 |
| GOh2029 | C538R | 0.46 |
| GOh2090 | F546M | 0.46 |
| GOh2077 | D239P | 0.46 |
| GOh2066 | D269N | 0.45 |
| GOh2026 | K389I | 0.45 |
| GOh2057 | S220T | 0.45 |
| GOh2001 | A195S | 0.45 |

TABLE 16
Table showing increased protein solubility of 11 purified
galactose oxidase mutants versus M3-5 control backbone.

| | Mutations | Soluble/Insoluble |
|---|---|---|
| Mutant No. | (on M3-5) | Ratio |
| M3-5 | Backbone | 0.58 |
| GOh1038 | F217D | 0.97 |
| GOh1013 | L537G | 0.94 |
| GOh1008 | S314C | 0.78 |
| GOh1032 | F217L | 0.78 |
| GOh1031 | F217T | 0.78 |
| GOh1006 | M353F | 0.77 |
| GOh1036 | F217A | 0.77 |
| GOh1021 | N268W | 0.76 |
| GOh1041 | F217K | 0.73 |
| GOh1035 | F217Y | 0.69 |
| GOh1034 | F217E | 0.66 |

### Analysis of Relaxed Enantioselectivity Profile of Galactose Oxidase Mutants

TABLE 17
Table showing relaxed enantioselectivity profile
of five purified enzymes (for substrate 152) versus
GOh1052 (F217A, N268W from M3-5) backbone.

| Mutant | Mutations | |
|---|---|---|
| No. | (on GOh1052) | R/S Ratio |
| GOh1052 | backbone | 1.67 |
| GOh2001 | A195S | 1.05 |
| GOh2025 | A346L | 1.25 |
| GOh2019 | T202D | 1.27 |
| GOh2014 | N341W | 1.35 |
| GOh2027 | Y352W | 1.45 |

TABLE 18
Table showing relaxed enantioselectivity profile of 15 purified enzymes
at F217 position (for substrate 128) versus M3-5 backbone.

| Mutant | Mutations | |
|---|---|---|
| No. | (on M3-5) | R/S Ratio |
| M3-5 | Backbone | 20.6 |
| GOh1036 | F217A | 1.6 |
| GOh1039 | F217G | 3.4 |
| GOh1016 | F217P | 4.1 |
| GOh1041 | F217K | 4.1 |
| GOh1042 | F217M | 4.7 |
| GOh1015 | F217V | 4.9 |
| GOh1038 | F217D | 6.0 |
| GOh1032 | F217L | 6.4 |
| GOh1030 | F217I | 6.7 |
| GOh1043 | F217R | 6.9 |
| GOh1020 | F217S | 7.0 |
| GOh1031 | F217T | 7.1 |
| GOh1037 | F217C | 8.6 |
| GOh1034 | F217E | 13.5 |
| GOh1040 | F217H | 19.8 |

The fold change ratios can be calculated as the benchmark R/S ratio divided by the mutant R/S ratio, which gives the fold decrease in enantio selectivity. For example, for GOh1036 benchmarked to M3-5, calculation is: 20.6 (M3-5 R/S ratio)/1.6 (GOh1036 R/S ratio) A v12.875 (round up to 12.9).

### Construction of the Combinatorial Library Targeting Generalist Activity and Stability of Galactose Oxidase Mutants.

Combinatorial library targeting generalist activity and stability was constructed from the following eight mutations (six amino acid mutation positions)
Generalist Activity: A195S, A195T, N259T, K389I
Stability: I28R, T153R, T153K, N341W

Purified galactose oxidase proteins from the combinatorial library have been found to surpass single mutants in the characteristics shown in Table 19.

TABLE 19

| | Single Mutant | Fold vs | Combinatorial | Fold vs |
|---|---|---|---|---|
| Charac- | (mutations on | GOh1052 | (mutations on | GOh1052 |
| teristic | GOh1052) | backbone | GOh1052) | backbone |
| 40° C. | GOh2045 | 1.85 | GOh3006 (I28R, | 2.18 |
| Activity | (I28R) | | K389I) | |
| Thermal | GOh2008 | 1.50 | GOh3006 (I28R, | 1.61 |
| Stability | (N259T) | | K389I) | |
| Protein | GOh2041 | 2.00 | GOh3002 | 10.09 |
| Expression | (F313R) | | (N341W, | |
| | | | Y352T) | |
| Solubility | GOh2082 | 2.82 | GOh3023 (I28R, | 4.57 |
| | (E218F) | | A195T, N341W, | |
| | | | K389I) | |

Synergistic mutations I28R and K389I (GOh3006) have been found to combine the desirable traits of both generalist activity and thermostability.

The present disclosure describes for the first time, galactose oxidase variants that convey superior enhancement in enzymatic activity towards bulky secondary alcohols and unactivated secondary alcohols.

The galactose oxidase variants of the present disclosure have the following advantages:
1. The galactose oxidase variants disclosed herein are capable of oxidising secondary alcohols and oxidising unactivated secondary alcohols.
2. The galactose oxidase variants disclosed herein has reduced enantioselectivity compared to modified galactose oxidase known in the art.
3. The galactose oxidase variants disclosed herein has improved enzyme solubility.
4. The galactose oxidase variants disclosed herein has improved protein expression.
5. The galactose oxidase variants disclosed herein has improved enzyme thermal stability.
6. The galactose oxidase variants disclosed herein possess overall improvement in enzyme properties such as activity, selectivity, expression, thermal stability and/or solubility.
7. The galactose oxidase variants disclosed herein can be used as a part of/all of the process for the manufacturing of APIs.
8. The galactose oxidase variants disclosed herein are useful in applications requiring the use of biocatalysts, e.g., bioremediation, cleaning, food production.

SEQUENCE LISTING

| SEQ ID | | |
|---|---|---|
| NO | Sequence Name | Sequence |
| 1 | GO01 = Wildtype | atgggcagcagccatcatcatcatcatcacagcagcggcctggtgccgcgcggca |
| | galactose oxidase | gccatatgggtaccgcttctgctccaattggttctgctatttccagaaacaattg |
| | from Fusarium | ggctgttacttgtgattctgctcaatctggtaatgaatgtaacaaggctattgat |
| | graminearum; | ggtaacaaggatactttttggcatactttctacggtgctaacggagatccaaaac |
| | has additional | cacctcacacttacactatcgatatgaagactactcaaaacgttaatggtttgtc |
| | His-tag | tatgttgcctagacaagatggtaaccaaaatggttggattggtagacatgaagtt |
| | at the C-terminal | tacttgtcttctgatggtactaattggggttctcctgttgcttccggttcttggt |
| | | ttgctgattctactactaaatactctaacttcgagactagaccagctagatatgt |
| | | tagattggttgctgttactgaagctaatggtcaaccttggacttctattgctgag |
| | | attaacgtttttcaggcttcttcttacactgctccacaacctggtttgggtagat |
| | | ggggtccaactattgatttgccaattgttcctgctgctgctgctattgagcctac |
| | | ttctggtagagttttgatgtggtcttcttatagaaatgatgcttttggtggttct |
| | | ccaggtggtattactttgacttcttcttgggacccatctactggtattgtttctg |
| | | atagaactgttactgttactaagcatgatatgttctgtccaggtatttctatgga |
| | | tggtaatggtcaaattgttgttactggtggtaacgatgctaagaaaacttctttg |
| | | tacgattcttcttctgattcttggattccaggtcctgatatgcaagttgctagag |
| | | gttatcaatcttctgctactatgtctgatggtagagtttttactattggtggttc |
| | | ttggtctggtggtgttttcgaaaagaacggagaggtttactctccatcttctaag |
| | | acttggacttctttgccaaatgctaaagttaaccctatgttgactgctgataagc |
| | | aaggtttgtatagatctgataatcacgcttggttgtttggttggaagaaaggttc |
| | | tgttttccaagctggtccttctactgctatgaactggtactatacttctggttct |
| | | ggagatgttaagtctgctggtaaaagacaatctaatagaggtgttgctccagatg |
| | | ctatgtgtggtaacgctgttatgtacgatgctgttaagggtaaaattttgacttt |
| | | cggtggttctcctgattatcaagattctgatgctactactaacgctcatatcatc |
| | | actttgggtgaaccaggtacttctcctaatactgtttttgcttctaacggtttgt |
| | | actttgctagaactttccacacttctgttgttttgccagatggttctactttcat |
| | | tactggtggtcaaagaagaggtattccttttgaggattctactccagttttcact |
| | | cctgaaatctacgttccagagcaagatactttctacaagcaaaaccctaattcta |
| | | tcgttagagtttaccattctatttctttgttgttgccagatggtagagttttcaa |
| | | cggtggtggtggtttgtgtggagattgtactactaaccacttcgatgctcaaatt |
| | | ttcactccaaactacttgtacaactctaacggtaacttggctactagacctaaga |
| | | tcactagaacttctactcaatctgttaaggttggtggtagaatcactatttctac |
| | | tgattcttctatcactaaggcttctttgatcagatatggtactgctactcacact |
| | | gttaatactgatcaaagaagaatcccattgactttgactaacaatggtggtaact |
| | | cttactcttttcaagttccatctgattctggtgttgctttgcctggttattggat |
| | | gttgttcgttatgaactctgctggtgttccttctgttgcttctactattagagtt |
| | | actcaagcggccgcactcgagcaccaccaccaccaccactga |
| 2 | GO01 = Wildtype | MGSSHHHHHHSSGLVPRGSHMGTASAPIGSAISRNNWAVTCDSAQSGNECNKAID |
| | galactose oxidase | GNKDTFWHTFYGANGDPKPPHTYTIDMKTTQNVNGLSMLPRQDGNQNGWIGRHEV |
| | from Fusarium | YLSSDGTNWGSPVASGSWFADSTTKYSNFETRPARYVRLVAVTEANGQPWTSIAE |
| | graminearum; | INVFQASSYTAPQPGLGRWGPTIDLPIVPAAAAIEPTSGRVLMWSSYRNDAFGGS |
| | same protein | PGGITLTSSWDPSTGIVSDRTVTVTKHDMFCPGISMDGNGQIVVTGGNDAKKTSL |
| | sequence as | YDSSSDSWIPGPDMQVARGYQSSATMSDGRVFTIGGSWSGGVFEKNGEVYSPSSK |
| | GO01b but has | TWTSLPNAKVNPMLTADKQGLYRSDNHAWLFGWKKGSVFQAGPSTAMNWYYTSGS |
| | additional His-tag | GDVKSAGKRQSNRGVAPDAMCGNAVMYDAVKGKILTFGGSPDYQDSDATTNAHII |
| | at the C-terminal | TLGEPGTSPNTVFASNGLYFARTFHTSVVLPDGSTFITGGQRRGIPFEDSTPVFT |
| | | PEIYVPEQDTFYKQNPNSIVRVYHSISLLLPDGRVFNGGGGLCGDCTTNHFDAQI |
| | | FTPNYLYNSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAALEHHHHHH |
| 3 | GO01b = E. coli | atgggcagcagccatcatcatcatcatcacagcagcggcctggtgccgcgcggca |
| | optimized version | gccatatgggtaccgcctctgcccctatcggctcagccatctcacgcaataactg |
| | of GO01; no C- | ggccgtaacgtgcgacagcgcgcagagcggcaacgagtgcaataaagccatcgac |
| | terminal His-tag | ggcaataaagacaccttctggcacacgttttatggcgcaaatggcgacccgaagc |
| | | caccgcatacctataccattgacatgaaaaccacccagaatgtgaacgggctgtc |
| | | catgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgttcggcggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atggagtggcggagtgtttgagaagaatggcgaagtctatagcccctcatccaaa |
| | | acgtggacgtcacttccgaacgcgaaggtgaatcccatgttaaccgccgacaaac |
| | | agggcctgtaccgcagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactaccaggacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgtgtatcactccatcagccttcttctcccggacggacgtgtctttaa |
| | | tggcggaggaggactgtgcggggactgcacgaccaatcattttgacgcgcagatc |
| | | tttaccccgaattatctgtataattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 4 | GO01b = E. coli | MGSSHHHHHHSSGLVPRGSHMGTASAPIGSAISRNNWAVTCDSAQSGNECNKAID |
| | optimized version | GNKDTFWHTFYGANGDPKPPHTYTIDMKTTQNVNGLSMLPRQDGNQNGWIGRHEV |
| | of GO01; no C- | YLSSDGTNWGSPVASGSWFADSTTKYSNFETRPARYVRLVAVTEANGQPWTSIAE |
| | terminal His-tag | INVFQASSYTAPQPGLGRWGPTIDLPIVPAAAAIEPTSGRVLMWSSYRNDAFGGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSWSGGVFEKNGEVYSPSSK |
| | | TWTSLPNAKVNPMLTADKQGLYRSDNHAWLFGWKKGSVFQAGPSTAMNWYYTSGS |
| | | GDVKSAGKRQSNRGVAPDAMCGNAVMYDAVKGKILTFGGSPDYQDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRVYHSISLLLPDGRVFNGGGGLCGDCTTNHFDAQI |
| | | FTPNYLYNSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 5 | GOh1001b = | atgggcagcagccatcatcatcatcatcacagcagcggcctggtgccgcgcggca |
| | GO01b + 8 | gccatatgggtaccgcctctgcccctatcggctcagccatcccgcgcaataactg |
| | mutations from | ggccgtaacgtgcgacagcgcgcagagcggcaacgagtgcaataaagccatcgac |
| | Turner's | ggcaataaagacaccttctggcacacgttttatggcgcaaatggcgacccgaagc |
| | ChemBiochem | caccgcatacctataccattgacatgaaaaccacccagaatgtgaacgggctgtc |
| | 2008 paper to | cgtgctgccgcgccaggacggcaatcagaacggatggatcggccgtcacgaggta |
| | change substrate | tatttaagctccgacggcaccaactggggatccccggtcgcgtcgggcagctggt |
| | specificity more | tcgccgacagcaccaccaagtattccaattttgaaacccgtcctgcacgctacgt |
| | towards | gcgcttagtggccgtgacagaggcgaacggccagccatggacctcaatcgccgaa |
| | secondary | atcaatgtattccaagccagctcctataccgcgccgcagccaggacttgggcgtt |
| | alcohols; | ggggcccgaccatcgacctgcctatcgtacccgcagccgcggcgattgaaccaac |
| | backbone for | gtcaggccgcgtactgatgtggagcagctaccgtaacgacgcgttcgaaggcagc |
| | Round 1; termed | ccgggcggcatcactctgacgtcgagttgggatccatccacgggcatcgtctccg |
| | as M3-5 variant | accgcaccgtgaccgtcacgaaacacgacatgttttgcccggggatcagcatgga |
| | for comparison to | cggcaacggccagatcgtcgtcacgggcggcaatgacgccaagaagacgagcctc |
| | Turner's paper | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 6 | GOh1001b = | MGSSHHHHHHSSGLVPRGSHMGTASAPIGSAIPRNNWAVTCDSAQSGNECNKAID |
| | GO01b + 8 | GNKDTFWHTFYGANGDPKPPHTYTIDMKTTQNVNGLSVLPRQDGNQNGWIGRHEV |
| | GOh1001b = | YLSSDGTNWGSPVASGSWFADSTTKYSNFETRPARYVRLVAVTEANGQPWTSIAE |
| | GO01b + 8 | INVFQASSYTAPQPGLGRWGITLTSSWDPSTGIVSDRTVTVTKHDMFCPGISMDG |
| | mutations from | NGQIVVTGGNDAKKTSLYDSSSDSWIPGPDMQVARGYQSSATMSDGRVFTIGGSF |
| | Turner's | SGGVFEKNGEVYSPSSKTWTSLPNAKVNPMLTADKQGLYMSDNHAWLFGWKKGSV |
| | ChemBiochem | FQAGPSTAMNWYYTSGSGDVKSAGKRQSNRGVAPDAMCGNAVMYDAVKGKILTFG |
| | 2008 paper to | GSPDYTDSDATTNAHIITLGEPGTSPNTVFASNGLYFARTFHTSVVLPDGSTFIT |
| | change substrate | GGQRRGIPFEDSTPVFTPEIYVPEQDTFYKQNPNSIVRAYHSISLLLPDGRVFNG |
| | specificity more | GGGLCGDCTTNHFDAQIFTPNYLYDSNGNLATRPKITRTSTQSVKVGGRITISTD |
| | towards | SSITKASLIRYGTATHTVNTDQRRIPLTLTNNGGNSYSFQVPSDSGVALPGYWML |
| | secondary | FVMNSAGVPSVASTIRVTQAAA |
| | alcohols; | |
| | backbone for | |
| | Round 1; termed | |
| | as M3-5 variant | |
| | for comparison to | |
| | Turner's paper | |
| 7 | GOh1006 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgttcgaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtactttagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 8 | GOh1006 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAFEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYFSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 9 | GOh1008 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgttcgaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | attttgtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 10 | GOh1008 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAFEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFCGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 11 | GOh1013 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgttcgaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggaggttgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 12 | GOh1013 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAFEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGGCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 13 | GOh1015 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggttgaaggcagc |
| | | ccgggggcatcactctgacgtcgagttgggatccatccacgggcatcgtctccga |
| | | ccgcaccgtgaccgtcacgaaacacgacatgttttgcccggggatcagcatggac |
| | | ggcaacggccagatcgtcgtcacgggcggcaatgacgccaagaagacgagcctct |
| | | atgacagtagcagcgacagttggatcccggggccggacatgcaggtggcacgcgg |
| | | gtaccagtccagcgcgaccatgtcagacggccgtgtctttacaatcggcggctca |
| | | tttagtggcggagtgtttgagaagaatggcgaagtctatagcccctcatccaaaa |
| | | cgtggacgtcacttccgaacgcgaaggtgaatcccatgttaaccgccgacaaaca |
| | | gggcctgtacatgagcgacaaccatgcgtggctgttcggctggaagaaggggagc |
| | | gtctttcaggcgggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 14 | GOh1015 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAVEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 15 | GOh1016 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgccggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 16 | GOh1016 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAPEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 17 | GOh1017 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgcaggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaaccc |
| 18 | GOh1017 | agtccgtcaaagtgggcggccgcattacgatcagtaccgacagcagcattaccaa |
| | | agcgtcactgattcgttacggcaccgcaacccatacagtcaacacggaccagcgc |
| | | cgtattccgttaaccctgaccaataacggtggcaattcgtatagcttccaggtac |
| | | cgtcggactcgggcgtggcgctgccaggatactggatgctgtttgtgatgaatag |
| | | cgcgggcgtcccgagcgtagcgagcacgatccgtgtgacccaggccgcagcttga |
| | | taaMGSSHHHHHHSSGLVPRGSHMGTASAPIGSAIPRNNWAVTCDSAQSGNECNK |
| | | AIDGNKDTFWHTFYGANGDPKPPHTYTIDMKTTQNVNGLSVLPRQDGNQNGWIGR |
| | | HEVYLSSDGTNWGSPVASGSWFADSTTKYSNFETRPARYVRLVAVTEANGQPWTS |
| | | IAEINVFQASSYTAPQPGLGRWGPTIDLPIVPAAAAIEPTSGRVLMWSSYRNDAQ |
| | | EGSPGGITLTSSWDPSTGIVSDRTVTVTKHDMFCPGISMDGNGQIVVTGGNDAKK |
| | | TSLYDSSSDSWIPGPDMQVARGYQSSATMSDGRVFTIGGSFSGGVFEKNGEVYSP |
| | | SSKTWTSLPNAKVNPMLTADKQGLYMSDNHAWLFGWKKGSVFQAGPSTAMNWYYT |
| | | SGSGDVKSAGKRQSNRGVAPDAMCGNAVMYDAVKGKILTFGGSPDYTDSDATTNA |
| | | HIITLGEPGTSPNTVFASNGLYFARTFHTSVVLPDGSTFITGGQRRGIPFEDSTP |
| | | VFTPEIYVPEQDTFYKQNPNSIVRAYHSISLLLPDGRVFNGGGGLCGDCTTNHFD |
| | | AQIFTPNYLYDSNGNLATRPKITRTSTQSVKVGGRITISTDSSITKASLIRYGTA |
| | | THTVNTDQRRIPLTLTNNGGNSYSFQVPSDSGVALPGYWMLFVMNSAGVPSVAST |
| | | IRVTQAAA |
| 19 | GOh1018 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgaatgaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tgggggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 20 | GOh1018 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDANEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 21 | GOh1020 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgtctgaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 22 | GOh1020 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDASEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 23 | GOh1021 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgttcgaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 24 | GOh1021 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAFEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 25 | GOh1025 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgttccctggcagc |
| | | ccgggggcatcactctgacgtcgagttgggatccatccacgggcatcgtctccga |
| | | ccgcaccgtgaccgtcacgaaacacgacatgttttgcccggggatcagcatggac |
| | | ggcaacggccagatcgtcgtcacgggcggcaatgacgccaagaagacgagcctct |
| | | atgacagtagcagcgacagttggatcccggggccggacatgcaggtggcacgcgg |
| | | gtaccagtccagcgcgaccatgtcagacggccgtgtctttacaatcggcggctca |
| | | tttagtggcggagtgtttgagaagaatggcgaagtctatagcccctcatccaaaa |
| | | cgtggacgtcacttccgaacgcgaaggtgaatcccatgttaaccgccgacaaaca |
| | | gggcctgtacatgagcgacaaccatgcgtggctgttcggctggaagaaggggagc |
| | | gtctttcagggggcccgtcgaccgcaatgaattggtattacacgtcaggaagcgg |
| | | ggacgtgaaatcagccggcaagcgccagtcgaaccgcggcgtggcgccggacgca |
| | | atgtgcggcaatgccgtgatgtatgacgcggtcaaaggcaagatcctgacatttg |
| | | gcgggagtccggactacaccgacagtgacgcgacaaccaatgcccacattattac |
| | | ccttggggagccggggacgagcccgaacacggtcttcgcgtccaatggcctctat |
| | | ttcgcgcgtacctttcatacgtcggtggtcctgccggacggatcaacctttatca |
| | | ccggcggacagcgccgtggcatccccttcgaagactcgacccccgtctttacacc |
| | | ggagatttatgtgccggaacaggacacgttttataaacagaatccgaactcgatt |
| | | gtacgcgcgtatcactccatcagccttcttctcccggacggacgtgtctttaatg |
| | | gcggaggaggactgtgcggggactgcacgaccaatcattttgacgcgcagatctt |
| | | taccccgaattatctgtatgattcaaatgggaatctcgcgacccgtccgaaaatt |
| | | acccgtacatcaacccagtccgtcaaagtgggcggccgcattacgatcagtaccg |
| | | acagcagcattaccaaagcgtcactgattcgttacggcaccgcaacccatacagt |
| | | caacacggaccagcgccgtattccgttaaccctgaccaataacggtggcaattcg |
| | | tatagcttccaggtaccgtcggactcgggcgtggcgctgccaggatactggatgc |
| | | tgtttgtgatgaatagcgcgggcgtcccgagcgtagcgagcacgatccgtgtgac |
| | | ccaggccgcagcttgataa |
| 26 | GOh1025 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAFPGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 27 | GOh1030 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgattgaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 28 | GOh1030 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAIEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 29 | GOh1031 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgacggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 30 | GOh1031 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDATEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 31 | GOh1032 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgttggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 32 | GOh1032 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDALEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 33 | GOh1034 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggaggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 34 | GOh1034 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAEEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 35 | GOh1035 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgtatgaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 36 | GOh1035 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAYEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 37 | GOh1036 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 38 | GOh1036 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 39 | GOh1037 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgtgtgaaggcagc |
| | | ccgggggcatcactctgacgtcgagttgggatccatccacgggcatcgtctccga |
| | | ccgcaccgtgaccgtcacgaaacacgacatgttttgcccggggatcagcatggac |
| | | ggcaacggccagatcgtcgtcacgggcggcaatgacgccaagaagacgagcctct |
| | | atgacagtagcagcgacagttggatcccggggccggacatgcaggtggcacgcgg |
| | | gtaccagtccagcgcgaccatgtcagacggccgtgtctttacaatcggcggctca |
| | | tttagtggcggagtgtttgagaagaatggcgaagtctatagcccctcatccaaaa |
| | | cgtggacgtcacttccgaacgcgaaggtgaatcccatgttaaccgccgacaaaca |
| | | gggcctgtacatgagcgacaaccatgcgtggctgttcggctggaagaaggggagc |
| | | gtctttcaggcgggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 40 | GOh1037 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDACEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 41 | GOh1038 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggatgaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 42 | GOh1038 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDADEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 43 | GOh1039 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgggtgaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 44 | GOh1039 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAGEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 45 | GOh1040 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgcatgaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 46 | GOh1040 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAHEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 47 | GOh1041 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgaaagaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcagggggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 48 | GOh1041 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAKEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 49 | GOh1042 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgatggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 50 | GOh1042 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAMEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 51 | GOh1043 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgcgtgaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 52 | GOh1043 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAREGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 53 | GOh1045 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgccacctggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 54 | GOh1045 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAPPGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 55 | GOh1046 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgccggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggagggtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 56 | GOh1046 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAPEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGGCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 57 | GOh1047 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgccgccaggcagc |
| | | ccgggggcatcactctgacgtcgagttgggatccatccacgggcatcgtctccga |
| | | ccgcaccgtgaccgtcacgaaacacgacatgttttgcccggggatcagcatggac |
| | | ggcaacggccagatcgtcgtcacgggcggcaatgacgccaagaagacgagcctct |
| | | atgacagtagcagcgacagttggatcccggggccggacatgcaggtggcacgcgg |
| | | gtaccagtccagcgcgaccatgtcagacggccgtgtctttacaatcggcggctca |
| | | tttagtggcggagtgtttgagaagaatggcgaagtctatagcccctcatccaaaa |
| | | cgtggacgtcacttccgaacgcgaaggtgaatcccatgttaaccgccgacaaaca |
| | | gggcctgtacatgagcgacaaccatgcgtggctgttcggctggaagaaggggagc |
| | | gtctttcaggcgggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggggacagcgccgtggcatccccttcgaagactcgacccccgtctttacacc |
| | | ggagatttatgtgccggaacaggacacgttttataaacagaatccgaactcgatt |
| | | gtacgcgcgtatcactccatcagccttcttctcccggacggacgtgtctttaatg |
| | | gcggaggaggagggtgcggggactgcacgaccaatcattttgacgcgcagatctt |
| | | taccccgaattatctgtatgattcaaatgggaatctcgcgacccgtccgaaaatt |
| | | acccgtacatcaacccagtccgtcaaagtgggcggccgcattacgatcagtaccg |
| | | acagcagcattaccaaagcgtcactgattcgttacggcaccgcaacccatacagt |
| | | caacacggaccagcgccgtattccgttaaccctgaccaataacggtggcaattcg |
| | | tatagcttccaggtaccgtcggactcgggcgtggcgctgccaggatactggatgc |
| | | tgtttgtgatgaatagcgcgggcgtcccgagcgtagcgagcacgatccgtgtgac |
| | | ccaggccgcagcttgataa |
| 58 | GOh1047 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAPPGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGGCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 59 | GOh1048 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggttccaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 60 | GOh1048 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAVPGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 61 | GOh1049 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggttgaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggagggtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 62 | GOh1049 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAVEGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGGCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 63 | GOh1050 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggttccaggcagc |
| | | ccgggggcatcactctgacgtcgagttgggatccatccacgggcatcgtctccga |
| | | ccgcaccgtgaccgtcacgaaacacgacatgttttgcccggggatcagcatggac |
| | | ggcaacggccagatcgtcgtcacgggcggcaatgacgccaagaagacgagcctct |
| | | atgacagtagcagcgacagttggatcccggggccggacatgcaggtggcacgcgg |
| | | gtaccagtccagcgcgaccatgtcagacggccgtgtctttacaatcggcggctca |
| | | tttagtggcggagtgtttgagaagaatggcgaagtctatagcccctcatccaaaa |
| | | cgtggacgtcacttccgaacgcgaaggtgaatcccatgttaaccgccgacaaaca |
| | | gggcctgtacatgagcgacaaccatgcgtggctgttcggctggaagaaggggagc |
| | | gtctttcaggcgggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggagggtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 64 | GOh1050 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAVPGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGGCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 65 | GOh1051 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgttcccaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggcaatgacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggagggtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 66 | GOh1051 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAFPGS |
| | | PGGITLTSSWDPSTGIVSDRTVTVTKHDMFCPGISMDGNGQIVVTGGNDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGGCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 67 | GOh1052 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tgggggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 68 | GOh1052 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 69 | GOh1053 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggggcatcactctgacgtcgagttgggatccatccacgggcatcgtctccga |
| | | ccgcaccgtgaccgtcacgaaacacgacatgttttgcccggggatcagcatggac |
| | | ggcaacggccagatcgtcgtcacgggcggctgggacgccaagaagacgagcctct |
| | | atgacagtagcagcgacagttggatcccggggccggacatgcaggtggcacgcgg |
| | | gtaccagtccagcgcgaccatgtcagacggccgtgtctttacaatcggcggctca |
| | | tttagtggcggagtgtttgagaagaatggcgaagtctatagcccctcatccaaaa |
| | | cgtggacgtcacttccgaacgcgaaggtgaatcccatgttaaccgccgacaaaca |
| | | gggcctgtactttagcgacaaccatgcgtggctgttcggctggaagaaggggagc |
| | | gtctttcagggggcccgtcgaccgcaatgaattggtattacacgtcaggaagcgg |
| | | ggacgtgaaatcagccggcaagcgccagtcgaaccgcggcgtggcgccggacgca |
| | | atgtgcggcaatgccgtgatgtatgacgcggtcaaaggcaagatcctgacatttg |
| | | gcgggagtccggactacaccgacagtgacgcgacaaccaatgcccacattattac |
| | | ccttggggagccggggacgagcccgaacacggtcttcgcgtccaatggcctctat |
| | | ttcgcgcgtacctttcatacgtcggtggtcctgccggacggatcaacctttatca |
| | | ccggcggacagcgccgtggcatccccttcgaagactcgacccccgtctttacacc |
| | | ggagatttatgtgccggaacaggacacgttttataaacagaatccgaactcgatt |
| | | gtacgcgcgtatcactccatcagccttcttctcccggacggacgtgtctttaatg |
| | | gcggaggaggactgtgcggggactgcacgaccaatcattttgacgcgcagatctt |
| | | taccccgaattatctgtatgattcaaatgggaatctcgcgacccgtccgaaaatt |
| | | acccgtacatcaacccagtccgtcaaagtgggcggccgcattacgatcagtaccg |
| | | acagcagcattaccaaagcgtcactgattcgttacggcaccgcaacccatacagt |
| | | caacacggaccagcgccgtattccgttaaccctgaccaataacggtggcaattcg |
| | | tatagcttccaggtaccgtcggactcgggcgtggcgctgccaggatactggatgc |
| | | tgtttgtgatgaatagcgcgggcgtcccgagcgtagcgagcacgatccgtgtgac |
| | | ccaggccgcagcttgataa |
| 70 | GOh1053 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYFSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 71 | GOh1054 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | attttgtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 72 | GOh1054 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFCGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 73 | GOh1055 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgggtgaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 74 | GOh1055 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAGEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 75 | GOh1056 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcgggtgaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtactttagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tgggggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 76 | GOh1056 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAGEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYFSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 77 | GOh2001 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccagcgccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 78 | GOh2001 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPSAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 79 | GOh2002 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcacccccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 80 | GOh2002 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPITPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 81 | GOh2008 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaccggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 82 | GOh2008 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGTGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 83 | GOh2010 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtcttcagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 84 | GOh2010 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVFSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 85 | GOh2011 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | cgtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 86 | GOh2011 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARAYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 87 | GOh2012 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccaccgccgcggcgattgaaccaac |
| | | gtcaggccgcgtactgatgtggagcagctaccgtaacgacgcggcggaaggcagc |
| | | ccgggggcatcactctgacgtcgagttgggatccatccacgggcatcgtctccga |
| | | ccgcaccgtgaccgtcacgaaacacgacatgttttgcccggggatcagcatggac |
| | | ggcaacggccagatcgtcgtcacgggcggctgggacgccaagaagacgagcctct |
| | | atgacagtagcagcgacagttggatcccggggccggacatgcaggtggcacgcgg |
| | | gtaccagtccagcgcgaccatgtcagacggccgtgtctttacaatcggcggctca |
| | | tttagtggcggagtgtttgagaagaatggcgaagtctatagcccctcatccaaaa |
| | | cgtggacgtcacttccgaacgcgaaggtgaatcccatgttaaccgccgacaaaca |
| | | gggcctgtacatgagcgacaaccatgcgtggctgttcggctggaagaaggggagc |
| | | gtctttcaggcgggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 88 | GOh2012 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPTAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 89 | GOh2013 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgaccatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 90 | GOh2013 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLTMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 91 | GOh2014 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgtggcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tgggggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 92 | GOh2014 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVWPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 93 | GOh2015 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagcctgtgcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 94 | GOh2015 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAACAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 95 | GOh2016 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | acggtaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 96 | GOh2016 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDGTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 97 | GOh2017 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcgattattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 98 | GOh2017 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAIIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 99 | GOh2018 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttggggttg |
| | | gggcccgaccatcgacctgcctatcgtacccgcagccgcggcgattgaaccaacg |
| | | tcaggccgcgtactgatgtggagcagctaccgtaacgacgcggcggaaggcagcc |
| | | cgggcggcatcactctgacgtcgagttgggatccatccacgggcatcgtctccga |
| | | ccgcaccgtgaccgtcacgaaacacgacatgttttgcccggggatcagcatggac |
| | | ggcaacggccagatcgtcgtcacgggcggctgggacgccaagaagacgagcctct |
| | | atgacagtagcagcgacagttggatcccggggccggacatgcaggtggcacgcgg |
| | | gtaccagtccagcgcgaccatgtcagacggccgtgtctttacaatcggcggctca |
| | | tttagtggcggagtgtttgagaagaatggcgaagtctatagcccctcatccaaaa |
| | | cgtggacgtcacttccgaacgcgaaggtgaatcccatgttaaccgccgacagcca |
| | | gggcctgtacatgagcgacaaccatgcgtggctgttcggctggaagaaggggagc |
| | | gtctttcaggcgggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 100 | GOh2018 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADSQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 101 | GOh2019 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaga |
| | | ttcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 102 | GOh2019 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPDSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 103 | GOh2020 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaca |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 104 | GOh2020 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPQSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 105 | GOh2021 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaat |
| | | ttcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 106 | GOh2021 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPISGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 107 | GOh2022 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaaa |
| | | atcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 108 | GOh2022 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPKSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 109 | GOh2023 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtatgtgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tgggggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 110 | GOh2023 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVCAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 111 | GOh2025 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccctggacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 112 | GOh2025 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTLDKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 113 | GOh2026 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgatctcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 114 | GOh2026 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVISAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 115 | GOh2027 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtggatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 116 | GOh2027 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLWMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 117 | GOh2028 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggggcatcactctgacgtcgagttgggatccatccacgggcatcgtctccga |
| | | ccgcaccgtgaccgtcacgaaacacgacatgttttgcccggggatcagcatggac |
| | | ggcaacggccagatcgtcgtcacgggcggctgggacgccaagaagacgagcctct |
| | | atgacagtagcagcgacagttggatcccggggccggacatgcaggtggcacgcgg |
| | | gtaccagtccagcgcgaccatgtcagacggccgtgtctttacaatcggcggctca |
| | | tttagtggcggagtgtttgagaagaatggcgaagtctatagcccctcatccaaaa |
| | | cgtggacgtcacttccgaacgcgaaggtgaatcccatgttaaccgccgacaaaca |
| | | gggcctgtacatgagcgacaaccatgcgtggctgttcggctggaagaaggggagc |
| | | gtctttcaggcgggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgaaaggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 118 | GOh2028 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLKGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 119 | GOh2029 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgcgtggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 120 | GOh2029 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLRGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 121 | GOh2030 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatggaatgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 122 | GOh2030 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMECPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 123 | GOh2031 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgtattgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 124 | GOh2031 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMYCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 125 | GOh2032 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaagcatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 126 | GOh2032 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDKHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 127 | GOh2033 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgaccgtcatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tgggggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 128 | GOh2033 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDRHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 129 | GOh2034 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgactggcatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 130 | GOh2034 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDWHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 131 | GOh2037 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatcccccgtgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 132 | GOh2037 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPREDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 133 | GOh2038 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttggggttg |
| | | gggcccgaccatcgacctgcctatcgtacccgcagccgcggcgattgaaccaacg |
| | | tcaggccgcgtactgatgtggagcagctaccgtaacgacgcggcggaaggcagcc |
| | | cgggcggcatcactctgacgtcgagttgggatccatccacgggcatcgtctccga |
| | | ccgcaccgtgaccgtcacgaaacacgacatgttttgcccggggatcagcatggac |
| | | ggcaacggccagatcgtcgtcacgggcggctgggacgccaagaagacgagcctct |
| | | atgacagtagcagcgacagttggatcccggggccggacatgcaggtggcacgcgg |
| | | gtaccagtccagcgcgaccatgtcagacggccgtgtctttacaatcggcggctca |
| | | tttagtggcggagtgtttgagaagaatggcgaagtctatagcccctcatccaaaa |
| | | cgtggacgtcacttccgaacgcgaaggtgaatcccatgttaaccgccgacaaaca |
| | | gggcctgtacatgagcgacaaccatgcgtggctgttcggctggaagaaggggagc |
| | | gtctttcaggcgggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactactgggacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 134 | GOh2038 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPSATMSDGRVFTIGGSFSGGVFEKNGEVYSPSSKTWTSLPNAKVNP |
| | | MLTADKQGLYMSDNHAWLFGWKKGSVFQAGPSTAMNWYYTSGSGDVKSAGKRQSN |
| | | RGVAPDAMCGNAVMYDAVKGKILTFGGSPDYWDSDATTNAHIITLGEPGTSPNTV |
| | | FASNGLYFARTFHTSVVLPDGSTFITGGQRRGIPFEDSTPVFTPEIYVPEQDTFY |
| | | KQNPNSIVRAYHSISLLLPDGRVFNGGGGLCGDCTTNHFDAQIFTPNYLYDSNGN |
| | | LATRPKITRTSTQSVKVGGRITISTDSSITKASLIRYGTATHTVNTDQRRIPLTL |
| | | TNNGGNSYSFQVPSDSGVALPGYWMLFVMNSAGVPSVASTIRVTQAAAGPDMQVA |
| | | RGYQS |
| 135 | GOh2039 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcgaaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 136 | GOh2039 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAKGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 137 | GOh2041 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | acgtagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 138 | GOh2041 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSRSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 139 | GOh2043 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggtatccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 140 | GOh2043 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWYPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 141 | GOh2044 | atgggcagcagccatcatcatcatcatcacagcagcggcctggtgccgcgcggca |
| | | gccatatgggtacccattctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcagggggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 142 | GOh2044 | MGSSHHHHHHSSGLVPRGSHMGTHSAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 143 | GOh2045 | atgggcagcagccatcatcatcatcatcacagcagcggcctggtgccgcgcggca |
| | | gccatatgggtaccgcctctgcccctcgtggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcagggggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 144 | GOh2045 | MGSSHHHHHHSSGLVPRGSHMGTASAPRGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 145 | GOh2046 | atgggcagcagccatcatcatcatcatcacagcagcggcctggtgccgcgcggca |
| | | gccatatgggtaccgcctctgcccctatgggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tgggggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 146 | GOh2046 | MGSSHHHHHHSSGLVPRGSHMGTASAPMGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 147 | GOh2047 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccaggatgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 148 | GOh2047 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQDVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 149 | GOh2048 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggagcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 150 | GOh2048 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWSPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 151 | GOh2050 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgccgtatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcagggggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 152 | GOh2050 | MGSSHHHHHHSSGLVPRGSHMGTASAPIGSAIPRNNWAVTCDSAQSGNECNKAID |
| | | GNKDTFWHTPYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 153 | GOh2053 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgcgtgaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 154 | GOh2053 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVREANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 155 | GOh2055 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccggtgcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tgggggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 156 | GOh2055 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAGAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 157 | GOh2056 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcagcggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 158 | GOh2056 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVSGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 159 | GOh2057 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcacc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 160 | GOh2057 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGT |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 161 | GOh2058 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtagcgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 162 | GOh2058 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRSDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 163 | GOh2061 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcgcacccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 164 | GOh2061 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGAPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 165 | GOh2064 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtcgtgacgcggcggaaggcagc |
| | | ccgggggcatcactctgacgtcgagttgggatccatccacgggcatcgtctccga |
| | | ccgcaccgtgaccgtcacgaaacacgacatgttttgcccggggatcagcatggac |
| | | ggcaacggccagatcgtcgtcacgggcggctgggacgccaagaagacgagcctct |
| | | atgacagtagcagcgacagttggatcccggggccggacatgcaggtggcacgcgg |
| | | gtaccagtccagcgcgaccatgtcagacggccgtgtctttacaatcggcggctca |
| | | tttagtggcggagtgtttgagaagaatggcgaagtctatagcccctcatccaaaa |
| | | cgtggacgtcacttccgaacgcgaaggtgaatcccatgttaaccgccgacaaaca |
| | | gggcctgtacatgagcgacaaccatgcgtggctgttcggctggaagaaggggagc |
| | | gtctttcaggcgggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 166 | GOh2064 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRRDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 167 | GOh2066 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggggcatcactctgacgtcgagttgggatccatccacgggcatcgtctccga |
| | | ccgcaccgtgaccgtcacgaaacacgacatgttttgcccggggatcagcatggac |
| | | ggcaacggccagatcgtcgtcacgggcggctggaatgccaagaagacgagcctct |
| | | atgacagtagcagcgacagttggatcccggggccggacatgcaggtggcacgcgg |
| | | gtaccagtccagcgcgaccatgtcagacggccgtgtctttacaatcggcggctca |
| | | tttagtggcggagtgtttgagaagaatggcgaagtctatagcccctcatccaaaa |
| | | cgtggacgtcacttccgaacgcgaaggtgaatcccatgttaaccgccgacaaaca |
| | | gggcctgtacatgagcgacaaccatgcgtggctgttcggctggaagaaggggagc |
| | | gtctttcaggcgggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcatcaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 168 | GOh2066 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWNAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 169 | GOh2067 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggccgtcccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 170 | GOh2067 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGRPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 171 | GOh2068 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcaaacccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 172 | GOh2068 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGKPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 173 | GOh2070 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggggcatcactctgacgtcgagttgggatccatccacgggcatcgtctccga |
| | | acgcaccgtgaccgtcacgaaacacgacatgttttgcccggggatcagcatggac |
| | | ggcaacggccagatcgtcgtcacgggcggctgggacgccaagaagacgagcctct |
| | | atgacagtagcagcgacagttggatcccggggccggacatgcaggtggcacgcgg |
| | | gtaccagtccagcgcgaccatgtcagacggccgtgtctttacaatcggcggctca |
| | | tttagtggcggagtgtttgagaagaatggcgaagtctatagcccctcatccaaaa |
| | | cgtggacgtcacttccgaacgcgaaggtgaatcccatgttaaccgccgacaaaca |
| | | gggcctgtacatgagcgacaaccatgcgtggctgttcggctggaagaaggggagc |
| | | gtctttcaggcgggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 174 | GOh2070 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSERTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 175 | GOh2071 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcgataactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tgggggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 176 | GOh2071 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGDNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 177 | GOh2072 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagccgtgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 178 | GOh2072 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSRDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 179 | GOh2073 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgatccgaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 180 | GOh2073 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDPNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 181 | GOh2074 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagcaatgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggggcatcactctgacgtcgagttgggatccatccacgggcatcgtctccga |
| | | ccgcaccgtgaccgtcacgaaacacgacatgttttgcccggggatcagcatggac |
| | | ggcaacggccagatcgtcgtcacgggcggctgggacgccaagaagacgagcctct |
| | | atgacagtagcagcgacagttggatcccggggccggacatgcaggtggcacgcgg |
| | | gtaccagtccagcgcgaccatgtcagacggccgtgtctttacaatcggcggctca |
| | | tttagtggcggagtgtttgagaagaatggcgaagtctatagcccctcatccaaaa |
| | | cgtggacgtcacttccgaacgcgaaggtgaatcccatgttaaccgccgacaaaca |
| | | gggcctgtacatgagcgacaaccatgcgtggctgttcggctggaagaaggggagc |
| | | gtctttcaggcgggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 182 | GOh2074 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSNDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 183 | GOh2075 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcactggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tgggggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | ccccggccgcagcttgataa |
| 184 | GOh2075 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TPAAA |
| 185 | GOh2076 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagccgttataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggggccgcattacgatcagtaccg |
| | | acagcagcattaccaaagcgtcactgattcgttacggcaccgcaacccatacagt |
| | | caacacggaccagcgccgtattccgttaaccctgaccaataacggtggcaattcg |
| | | tatagcttccaggtaccgtcggactcgggcgtggcgctgccaggatactggatgc |
| | | tgtttgtgatgaatagcgcgggcgtcccgagcgtagcgagcacgatccgtgtgac |
| | | ccaggccgcagcttgataa |
| 186 | GOh2076 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASRYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 187 | GOh2077 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccc |
| | | cgcgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 188 | GOh2077 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSPRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 189 | GOh2078 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccgaatcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggggcatcactctgacgtcgagttgggatccatccacgggcatcgtctccga |
| | | ccgcaccgtgaccgtcacgaaacacgacatgttttgcccggggatcagcatggac |
| | | ggcaacggccagatcgtcgtcacgggcggctgggacgccaagaagacgagcctct |
| | | atgacagtagcagcgacagttggatcccggggccggacatgcaggtggcacgcgg |
| | | gtaccagtccagcgcgaccatgtcagacggccgtgtctttacaatcggcggctca |
| | | tttagtggcggagtgtttgagaagaatggcgaagtctatagcccctcatccaaaa |
| | | cgtggacgtcacttccgaacgcgaaggtgaatcccatgttaaccgccgacaaaca |
| | | gggcctgtacatgagcgacaaccatgcgtggctgttcggctggaagaaggggagc |
| | | gtctttcaggcgggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 190 | GOh2078 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQAESYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 191 | GOh2079 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tgggggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | ccgatgccgcagcttgataa |
| 192 | GOh2079 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TDAAA |
| 193 | GOh2080 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagcaaatataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tgggggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 194 | GOh2080 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASKYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 195 | GOh2081 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgttgccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 196 | GOh2081 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPVAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 197 | GOh2082 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcgtttggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 198 | GOh2082 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAFGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 199 | GOh2083 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactcagacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 200 | GOh2083 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITQTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 201 | GOh2084 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggctggactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 202 | GOh2084 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGWTLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 203 | GOh2085 | atgggcagcagccatcatcatcatcatcacagcagcggcctggtgccgcgcggca |
| | | gccatatgggtaccgcctctcgtcctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggggcatcactctgacgtcgagttgggatccatccacgggcatcgtctccga |
| | | ccgcaccgtgaccgtcacgaaacacgacatgttttgcccggggatcagcatggac |
| | | ggcaacggccagatcgtcgtcacgggcggctgggacgccaagaagacgagcctct |
| | | atgacagtagcagcgacagttggatcccggggccggacatgcaggtggcacgcgg |
| | | gtaccagtccagcgcgaccatgtcagacggccgtgtctttacaatcggcggctca |
| | | tttagtggcggagtgtttgagaagaatggcgaagtctatagcccctcatccaaaa |
| | | cgtggacgtcacttccgaacgcgaaggtgaatcccatgttaaccgccgacaaaca |
| | | gggcctgtacatgagcgacaaccatgcgtggctgttcggctggaagaaggggagc |
| | | gtctttcaggcgggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 204 | GOh2085 | MGSSHHHHHHSSGLVPRGSHMGTASRPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 205 | GOh2086 | atgggcagcagccatcatcatcatcatcacagcagcggcctggtgccgcgcggca |
| | | gccatatgggtaccgcctctgccccttttggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 206 | GOh2086 | MGSSHHHHHHSSGLVPRGSHMGTASAPFGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 207 | GOh2087 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcgtatggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 208 | GOh2087 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAYGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 209 | GOh2088 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactgttacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 210 | GOh2088 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITVTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 211 | GOh2089 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccc |
| | | agcgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 212 | GOh2089 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSQRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 213 | GOh2090 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcacatggacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 214 | GOh2090 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHMDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 215 | GOh2091 | atgggcagcagccatcatcatcatcatcacagcagcggcctggtgccgcgcggca |
| | | gccatatgggtaccaattctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 216 | GOh2091 | MGSSHHHHHHSSGLVPRGSHMGTNSAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 217 | GOh2092 | atgggcagcagccatcatcatcatcatcacagcagcggcctggtgccgcgcggca |
| | | gccatatgggtaccgcctctcatcctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 218 | GOh2092 | MGSSHHHHHHSSGLVPRGSHMGTASHPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 219 | GOh2093 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgtatgggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 220 | GOh2093 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRMGTATHT |
| | | VNTDQRRIPLTLTNNGGNSYSFQVPSDSGVALPGYWMLFVMNSAGVPSVASTIRV |
| | | TQAAA |
| 221 | GOh2094 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacaatcagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 222 | GOh2094 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANNQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 223 | GOh2097 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcggatagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 224 | GOh2097 | MGSSHHHHHHSSGLVPRGSHMGTASAPIGSAIPRNNWAVTCDSADSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 225 | GOh2098 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcggaaagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 226 | GOh2098 | MGSSHHHHHHSSGLVPRGSHMGTASAPIGSAIPRNNWAVTCDSAESGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 227 | GOh2099 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgaatagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 228 | GOh2099 | MGSSHHHHHHSSGLVPRGSHMGTASAPIGSAIPRNNWAVTCDSANSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 229 | GOh2100 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacaaatgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 230 | GOh2100 | MGSSHHHHHHSSGLVPRGSHMGTASAPIGSAIPRNNWAVTCDSAQSGNKCNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 231 | GOh2101 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcagatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 232 | GOh2101 | MGSSHHHHHHSSGLVPRGSHMGTASAPIGSAIPRNNWAVTCDSAQSGNECNKAID |
| | | GNKDTFWHTFYGADGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 233 | GOh2102 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcagaaggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 234 | GOh2102 | MGSSHHHHHHSSGLVPRGSHMGTASAPIGSAIPRNNWAVTCDSAQSGNECNKAID |
| | | GNKDTFWHTFYGAEGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 235 | GOh2103 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcatttggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 236 | GOh2103 | MGSSHHHHHHSSGLVPRGSHMGTASAPIGSAIPRNNWAVTCDSAQSGNECNKAID |
| | | GNKDTFWHTFYGAFGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 237 | GOh2104 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcacatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 238 | GOh2104 | MGSSHHHHHHSSGLVPRGSHMGTASAPIGSAIPRNNWAVTCDSAQSGNECNKAID |
| | | GNKDTFWHTFYGAHGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 239 | GOh2105 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcacttggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 240 | GOh2105 | MGSSHHHHHHSSGLVPRGSHMGTASAPIGSAIPRNNWAVTCDSAQSGNECNKAID |
| | | GNKDTFWHTFYGALGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 241 | GOh2106 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaatgggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 242 | GOh2106 | MGSSHHHHHHSSGLVPRGSHMGTASAPIGSAIPRNNWAVTCDSAQSGNECNKAID |
| | | GNKDTFWHTFYGAMGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 243 | GOh2107 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaacaggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 244 | GOh2107 | MGSSHHHHHHSSGLVPRGSHMGTASAPIGSAIPRNNWAVTCDSAQSGNECNKAID |
| | | GNKDTFWHTFYGATGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 245 | GOh2108 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcatggggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 246 | GOh2108 | MGSSHHHHHHSSGLVPRGSHMGTASAPIGSAIPRNNWAVTCDSAQSGNECNKAID |
| | | GNKDTFWHTFYGAWGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 247 | GOh2109 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcatatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 248 | GOh2109 | MGSSHHHHHHSSGLVPRGSHMGTASAPIGSAIPRNNWAVTCDSAQSGNECNKAID |
| | | GNKDTFWHTFYGAYGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 249 | GOh2110 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggaggtatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 250 | GOh2110 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGGIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 251 | GOh2111 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggaaatatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 252 | GOh2111 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGNIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 253 | GOh2114 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctatgaagcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 254 | GOh2114 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYEAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 255 | GOh2115 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctatcatgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 256 | GOh2115 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYHAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 257 | GOh2116 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctatattgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 258 | GOh2116 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYIAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 259 | GOh2117 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataaagcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tgggggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggggccgcattacgatcagtaccg |
| | | acagcagcattaccaaagcgtcactgattcgttacggcaccgcaacccatacagt |
| | | caacacggaccagcgccgtattccgttaaccctgaccaataacggtggcaattcg |
| | | tatagcttccaggtaccgtcggactcgggcgtggcgctgccaggatactggatgc |
| | | tgtttgtgatgaatagcgcgggcgtcccgagcgtagcgagcacgatccgtgtgac |
| | | ccaggccgcagcttgataa |
| 260 | GOh2117 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYKAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 261 | GOh2119 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggatccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 262 | GOh2119 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVDPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 263 | GOh3001 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccaccgccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtggatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 264 | GOh3001 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPTAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLWMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 265 | GOh3002 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgtggcccatgttaaccgccgacaaac |
| | | agggcctgaccatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 266 | GOh3002 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVWPMLTADKQGLTMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 267 | GOh3003 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcgattattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaccggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcagggggcccgtcgaccgcaatgaattggtattacacgtcaggaagcg |
| | | gggacgtgaaatcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 268 | GOh3003 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAIIEPTSGRVLMWSSYRNDAAEGS |
| | | PGGITLTSSWDPSTGIVSDRTVTVTKHDMFCPGISMDGTGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 269 | GOh3004 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccagcgccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgtggcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 270 | GOh3004 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPSAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVWPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 271 | GOh3005 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgaaagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccagcgccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 272 | GOh3005 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVKEANGQPWTSIAE |
| | | INVFQASSYTAPQPGLGRWGPTIDLPIVPSAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 273 | GOh3006 | atgggcagcagccatcatcatcatcatcacagcagcggcctggtgccgcgcggca |
| | | gccatatgggtaccgcctctgcccctcgtggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcagggggcccgtcgaccgcaatgaattggtattacacgtcaggaagcg |
| | | gggacgtgatctcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 274 | GOh3006 | MGSSHHHHHHSSGLVPRGSHMGTASAPRGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVISAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 275 | GOh3007 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgcgtgaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccaccgccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 276 | GOh3007 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVREANGQPWTSIAE |
| | | INVFQASSYTAPQPGLGRWGPTIDLPIVPTAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 277 | GOh3008 | atgggcagcagccatcatcatcatcatcacagcagcggcctggtgccgcgcggca |
| | | gccatatgggtaccgcctctgcccctcgtggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgcgtgaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcgattattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgtggcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 278 | GOh3008 | MGSSHHHHHHSSGLVPRGSHMGTASAPRGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVREANGQPWTSIAE |
| | | INVFQASSYTAPQPGLGRWGPTIDLPIVPAAAIIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVWPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 279 | GOh3009 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccaccgccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaccggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgtggcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 280 | GOh3009 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPTAAAIEPTSGRVLMWSSYRNDAAEGS |
| | | PGGITLTSSWDPSTGIVSDRTVTVTKHDMFCPGISMDGTGQIVVTGGWDAKKTSL |
| | | YDSSSDSWIPGPDMQVARGYQSSATMSDGRVFTIGGSFSGGVFEKNGEVYSPSSK |
| | | TWTSLPNAKVWPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 281 | GOh3013 | atgggcagcagccatcatcatcatcatcacagcagcggcctggtgccgcgcggca |
| | | gccatatgggtaccgcctctgcccctcgtggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccaccgccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaccggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgatctcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 282 | GOh3013 | MGSSHHHHHHSSGLVPRGSHMGTASAPRGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPTAAAIEPTSGRVLMWSSYRNDAAEGS |
| | | PGGITLTSSWDPSTGIVSDRTVTVTKHDMFCPGISMDGTGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVISAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 283 | GOh3015 | atgggcagcagccatcatcatcatcatcacagcagcggcctggtgccgcgcggca |
| | | gccatatgggtaccgcctctgcccctcgtggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaccggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgtggcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 284 | GOh3015 | MGSSHHHHHHSSGLVPRGSHMGTASAPRGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGTGQIVVTGGWDAKKTSL |
| | | YDSSSDSWIPGPDMQVARGYQSSATMSDGRVFTIGGSFSGGVFEKNGEVYSPSSK |
| | | TWTSLPNAKVWPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 285 | GOh3018 | atgggcagcagccatcatcatcatcatcacagcagcggcctggtgccgcgcggca |
| | | gccatatgggtaccgcctctgcccctcgtggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccagcgccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgatctcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 286 | GOh3018 | MGSSHHHHHHSSGLVPRGSHMGTASAPRGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPSAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVISAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 287 | GOh3019 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccaccgccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgtggcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 288 | GOh3019 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPTAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVWPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 289 | GOh3023 | atgggcagcagccatcatcatcatcatcacagcagcggcctggtgccgcgcggca |
| | | gccatatgggtaccgcctctgcccctcgtggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccaccgccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgtggcccatgttaaccgccgacaaac |
| | | agggcctgtacatgagcgacaaccatgcgtggctgttcggctggaagaaggggag |
| | | cgtctttcagggggcccgtcgaccgcaatgaattggtattacacgtcaggaagcg |
| | | gggacgtgatctcagccggcaagcgccagtcgaaccgcggcgtggcgccggacgc |
| | | aatgtgcggcaatgccgtgatgtatgacgcggtcaaaggcaagatcctgacattt |
| | | ggcgggagtccggactacaccgacagtgacgcgacaaccaatgcccacattatta |
| | | cccttggggagccggggacgagcccgaacacggtcttcgcgtccaatggcctcta |
| | | tttcgcgcgtacctttcatacgtcggtggtcctgccggacggatcaacctttatc |
| | | accggcggacagcgccgtggcatccccttcgaagactcgacccccgtctttacac |
| | | cggagatttatgtgccggaacaggacacgttttataaacagaatccgaactcgat |
| | | tgtacgcgcgtatcactccatcagccttcttctcccggacggacgtgtctttaat |
| | | ggcggaggaggactgtgcggggactgcacgaccaatcattttgacgcgcagatct |
| | | ttaccccgaattatctgtatgattcaaatgggaatctcgcgacccgtccgaaaat |
| | | tacccgtacatcaacccagtccgtcaaagtgggcggccgcattacgatcagtacc |
| | | gacagcagcattaccaaagcgtcactgattcgttacggcaccgcaacccatacag |
| | | tcaacacggaccagcgccgtattccgttaaccctgaccaataacggtggcaattc |
| | | gtatagcttccaggtaccgtcggactcgggcgtggcgctgccaggatactggatg |
| | | ctgtttgtgatgaatagcgcgggcgtcccgagcgtagcgagcacgatccgtgtga |
| | | cccaggccgcagcttgataa |
| 290 | GOh3023 | MGSSHHHHHHSSGLVPRGSHMGTASAPRGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPTAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVWPMLTADKQGLYMSDNHAWLFGWKKGSVFQAGPSTAMNWYYTSGS |
| | | GDVISAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 291 | GOh3026 | atgggcagcagccatcatcatcatcatcacagcagcggcctggtgccgcgcggca |
| | | gccatatgggtaccgcctctgcccctcgtggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccaccgccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaccggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgaatcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccgggggcgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 292 | GOh3026 | MGSSHHHHHHSSGLVPRGSHMGTASAPRGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPTAAAIEPTSGRVLMWSSYRNDAAEGS |
| | | PGGITLTSSWDPSTGIVSDRTVTVTKHDMFCPGISMDGTGQIVVTGGWDAKKTSL |
| | | YDSSSDSWIPGPDMQVARGYQSSATMSDGRVFTIGGSFSGGVFEKNGEVYSPSSK |
| | | TWTSLPNAKVNPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGASPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 293 | GOh3032 | atgggcagcagccatcatcatcatcatcacagcagcggcctggtgccgcgcggca |
| | | gccatatgggtaccgcctctgcccctcgtggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccagcgccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaccggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgtggcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 294 | GOh3032 | MGSSHHHHHHSSGLVPRGSHMGTASAPRGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPSAAAIEPTSGRVLMWSSYRNDAAEGS |
| | | PGGITLTSSWDPSTGIVSDRTVTVTKHDMFCPGISMDGTGQIVVTGGWDAKKTSL |
| | | YDSSSDSWIPGPDMQVARGYQSSATMSDGRVFTIGGSFSGGVFEKNGEVYSPSSK |
| | | TWTSLPNAKVWPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 295 | GOh3038 | atgggcagcagccatcatcatcatcatcacagcagcggcctggtgccgcgcggca |
| | | gccatatgggtaccgcctctgcccctcgtggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgacagaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccaccgccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaccggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgtggcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgatctcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 296 | GOh3038 | MGSSHHHHHHSSGLVPRGSHMGTASAPRGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVTEANGQPWTSIAE |
| | | INVFQASSYTAPQPGLGRWGPTIDLPIVPTAAAIEPTSGRVLMWSSYRNDAAEGS |
| | | PGGITLTSSWDPSTGIVSDRTVTVTKHDMFCPGISMDGTGQIVVTGGWDAKKTSL |
| | | YDSSSDSWIPGPDMQVARGYQSSATMSDGRVFTIGGSFSGGVFEKNGEVYSPSSK |
| | | TWTSLPNAKVWPMLTADKQGLYMSDNHAWLFGWKKGSVFQAGPSTAMNWYYTSGS |
| | | GDVISAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |
| 297 | GOh3049 | atgggcagcagccatcatcatcatcatcacagcagcggcctggtgccgcgcggca |
| | | gccatatgggtaccgcctctgcccctatcggctcagccatcccgcgcaataactg |
| | | ggccgtaacgtgcgacagcgcgcagagcggcaacgagtgcaataaagccatcgac |
| | | ggcaataaagacaccttctggcacacgttttatggcgcaaatggcgacccgaagc |
| | | caccgcatacctataccattgacatgaaaaccacccagaatgtgaacgggctgtc |
| | | cgtgctgccgcgccaggacggcaatcagaacggatggatcggccgtcacgaggta |
| | | tatttaagctccgacggcaccaactggggatccccggtcgcgtcgggcagctggt |
| | | tcgccgacagcaccaccaagtattccaattttgaaacccgtcctgcacgctacgt |
| | | gcgcttagtggccgtgcgtgaggcgaacggccagccatggacctcaatcgccgaa |
| | | atcaatgtattccaagccagctcctataccgcgccgcagccaggacttgggcgtt |
| | | ggggcccgaccatcgacctgcctatcgtacccgcagccgcggcgattgaaccaac |
| | | gtcaggccgcgtactgatgtggagcagctaccgtaacgacgcggcggaaggcagc |
| | | ccgggcggcatcactctgacgtcgagttgggatccatccacgggcatcgtctccg |
| | | accgcaccgtgaccgtcacgaaacacgacatgttttgcccggggatcagcatgga |
| | | cggcaacggccagatcgtcgtcacgggcggctgggacgccaagaagacgagcctc |
| | | tatgacagtagcagcgacagttggatcccggggccggacatgcaggtggcacgcg |
| | | ggtaccagtccagcgcgaccatgtcagacggccgtgtctttacaatcggcggctc |
| | | atttagtggcggagtgtttgagaagaatggcgaagtctatagcccctcatccaaa |
| | | acgtggacgtcacttccgaacgcgaaggtgtggcccatgttaaccgccgacaaac |
| | | agggcctgtacatgagcgacaaccatgcgtggctgttcggctggaagaaggggag |
| | | cgtctttcaggcgggcccgtcgaccgcaatgaattggtattacacgtcaggaagc |
| | | ggggacgtgaaatcagccggcaagcgccagtcgaaccgcggcgtggcgccggacg |
| | | caatgtgcggcaatgccgtgatgtatgacgcggtcaaaggcaagatcctgacatt |
| | | tggcgggagtccggactacaccgacagtgacgcgacaaccaatgcccacattatt |
| | | acccttggggagccggggacgagcccgaacacggtcttcgcgtccaatggcctct |
| | | atttcgcgcgtacctttcatacgtcggtggtcctgccggacggatcaacctttat |
| | | caccggcggacagcgccgtggcatccccttcgaagactcgacccccgtctttaca |
| | | ccggagatttatgtgccggaacaggacacgttttataaacagaatccgaactcga |
| | | ttgtacgcgcgtatcactccatcagccttcttctcccggacggacgtgtctttaa |
| | | tggcggaggaggactgtgcggggactgcacgaccaatcattttgacgcgcagatc |
| | | tttaccccgaattatctgtatgattcaaatgggaatctcgcgacccgtccgaaaa |
| | | ttacccgtacatcaacccagtccgtcaaagtgggcggccgcattacgatcagtac |
| | | cgacagcagcattaccaaagcgtcactgattcgttacggcaccgcaacccataca |
| | | gtcaacacggaccagcgccgtattccgttaaccctgaccaataacggtggcaatt |
| | | cgtatagcttccaggtaccgtcggactcgggcgtggcgctgccaggatactggat |
| | | gctgtttgtgatgaatagcgcgggcgtcccgagcgtagcgagcacgatccgtgtg |
| | | acccaggccgcagcttgataa |
| 298 | GOh3049 | MGSSHHHHHHSSGLVPRGSHMGTASAPIGSAIPRNNWAVTCDSAQSGNECNKAID |
| | | GNKDTFWHTFYGANGDPKPPHTYTIDMKTTQNVNGLSVLPRQDGNQNGWIGRHEV |
| | | YLSSDGTNWGSPVASGSWFADSTTKYSNFETRPARYVRLVAVREANGQPWTSIAE |
| | | INVFQASSYTAPQPGLGRWGPTIDLPIVPAAAAIEPTSGRVLMWSSYRNDAAEGS |
| | | PGGITLTSSWDPSTGIVSDRTVTVTKHDMFCPGISMDGNGQIVVTGGWDAKKTSL |
| | | YDSSSDSWIPGPDMQVARGYQSSATMSDGRVFTIGGSFSGGVFEKNGEVYSPSSK |
| | | TWTSLPNAKVWPMLTADKQGLYMSDNHAWLFGWKKGSVFQAGPSTAMNWYYTSGS |
| | | GDVKSAGKRQSNRGVAPDAMCGNAVMYDAVKGKILTFGGSPDYTDSDATTNAHII |
| | | TLGEPGTSPNTVFASNGLYFARTFHTSVVLPDGSTFITGGQRRGIPFEDSTPVFT |
| | | PEIYVPEQDTFYKQNPNSIVRAYHSISLLLPDGRVFNGGGGLCGDCTTNHFDAQI |
| | | FTPNYLYDSNGNLATRPKITRTSTQSVKVGGRITISTDSSITKASLIRYGTATHT |
| | | VNTDQRRIPLTLTNNGGNSYSFQVPSDSGVALPGYWMLFVMNSAGVPSVASTIRV |
| | | TQAAA |

1. A modified galactose oxidase comprising a polypeptide sequence having at least 85%, 86%, 87%, 88%, 89%, 90%, 91%, 92%, 93%, 94%, 95%, 96%, 97%, 98%, or 99% sequence identity to SEQ ID NO: 6,
wherein SEQ ID NO: 6 is a polypeptide sequence of galactose oxidase variant M3-5, wherein the galactose oxidase variant M3-5 comprises mutations of S33P, M93V, G218E, W313F, R353M, Q429T, V517A, and N558D relative to the wildtype galactose oxidase from Fusarium graminearum (SEQ ID NO: 4),
and wherein the polypeptide sequence of the modified galactose oxidase further comprises at least one mutation at one or more positions selected from the group consisting of 24, 26, 28, 45, 49, 65, 69, 104, 114, 117, 153, 157, 172, 173, 175, 193,194, 195, 197, 198,202, 214, 217, 218, 220, 224, 226, 239, 240, 250, 259, 265, 268, 269, 284, 294, 313, 314, 325, 341, 346, 348, 352, 353, 356, 389, 401, 429, 447, 485, 487, 537, 538, 546, 559, 576, 599, and 662, wherein the position of the mutation is numbered with reference to SEQ ID NO: 6.

2. The modified galactose oxidase of claim 1, wherein the mutation is selected from the group consisting of A24H, A24N, A26H, A26R, I28F, I28M, I28R, Q45D, Q45E, Q45N, E49K, F65P, N69D, N69E, N69F, N69H, N69L, N69M, N69T, N69W, N69Y, W104G, W104N, S114N, S114R, T117D, T153K, T153R, G157N, S172E, S173K, S173R, T175E, T175H, T175I, T175K, V193T, P194C, A195S, A195T, A195V, A197C, A197G, A198I, T202D, T202I, T202K, T202Q, N214R, N214S, F217A, F217C, F217D, F217E, F217G, F217H, F217I, F217K, F217L, F217M, F217N, F217P, F217Q, F217R, F217S, F217T, F217V, F217Y, E218F, E218K, E218P, E218Y, S220T, I224W, L226Q, L226V, D239E, D239P, D239Q, R240G, F250E, F250Y, N259T, T265S, N268W, D269N, I284S, I284Y, G294A, F313R, S314C, Y325F, N341W, A346L, K348S, Y352T, Y352W, M353F, N356K, N356R, N356W, K389I, A401 D, T429W, T447A, I485A, I485K, I485R, F487R, L537G, C538K, C538R, F546M, S559P, S576D, Y599M, Q662D, and Q662P, or the mutation is selected from the group consisting of I28R, A24H, A195S, A195T, A195V, A198I, T202I, F217A, E218F, E218K, L226Q, D239E, N259T, N268W, S314C, Y352W, Y599M, and combinations thereof.

3. (canceled)

4. The modified galactose oxidase of claim 1, wherein the mutation is selected from the group consisting of:
M353F;
S314C;
L537G;
F217V;
F217P;
F217Q;
F217N;
F217S;
N268W;
E218P;
F217I;
F217T;
F217L;
F217E;
F217Y;
F217A;
F217C;
F217D;
F217G;
F217H;
F217K;
F217M;
F217R;
a combination of F217P and E218P;
a combination of F217P and L537G;
a combination of F217P, E218P and L537G;
a combination of F217V and E218P;
a combination of F217V and L537G;
a combination of F217V, E218P and L537G;
a combination of E218P and L537G;
a combination of F217A and N268W;
a combination of F217A, N268W and M353F;
a combination of F217A, N268W and S314C;
a combination of F217G and N268W;
a combination of F217G, N268W and M353F;
a combination of F217A, N268W and A195S;
a combination of F217A, N268W and V193T;
a combination of F217A, N268W and N259T;
a combination of F217A, N268W and Y325F;
a combination of F217A, N268W and G294A;
a combination of F217A, N268W and A195T;
a combination of F217A, N268W and Y352T;
a combination of F217A, N268W and N341W;
a combination of F217A, N268W and A197C;
a combination of F217A, N268W and R240G;
a combination of F217A, N268W and A198I;
a combination of F217A, N268W and K348S;
a combination of F217A, N268W and T202D;
a combination of F217A, N268W and T202Q;
a combination of F217A, N268W and T202I;
a combination of F217A, N268W and T202K;
a combination of F217A, N268W and P194C;
a combination of F217A, N268W and A346L;
a combination of F217A, N268W and K389I;
a combination of F217A, N268W and Y352W;
a combination of F217A, N268W and C538K;
a combination of F217A, N268W and C538R;
a combination of F217A, N268W and F250E;
a combination of F217A, N268W and F250Y;
a combination of F217A, N268W and N356K;
a combination of F217A, N268W and N356R;
a combination of F217A, N268W and N356W;
a combination of F217A, N268W and F487R;
a combination of F217A, N268W and T429W;
a combination of F217A, N268W and E218K;
a combination of F217A, N268W and F313R;
a combination of F217A, N268W and I284Y;
a combination of F217A, N268W and A24H;
a combination of F217A, N268W and I28R;
a combination of F217A, N268W and I28M;
a combination of F217A, N268W and S576D;
a combination of F217A, N268W and I284S;
a combination of F217A, N268W and F65P;
a combination of F217A, N268W and T153R;
a combination of F217A, N268W and A197G;
a combination of F217A, N268W and T265S;
a combination of F217A, N268W and S220T;
a combination of F217A, N268W and N214S;
a combination of F217A, N268W and I485A;
a combination of F217A, N268W and N214R;
a combination of F217A, N268W and D269N;
a combination of F217A, N268W and I485R;
a combination of F217A, N268W and I485K;
a combination of F217A, N268W and D239E;
a combination of F217A, N268W and T117D;
a combination of F217A, N268W and S114R;
a combination of F217A, N268W and S559P;
a combination of F217A, N268W and S114N;
a combination of F217A, N268W and Q662P;
a combination of F217A, N268W and S173R;
a combination of F217A, N268W and D239P;
a combination of F217A, N268W and S172E;
a combination of F217A, N268W and Q662D;
a combination of F217A, N268W and S173K;
a combination of F217A, N268W and A195V;
a combination of F217A, N268W and E218F;
a combination of F217A, N268W and L226Q;
a combination of F217A, N268W and I224W;
a combination of F217A, N268W and A26R;
a combination of F217A, N268W and I28F;
a combination of F217A, N268W and E218Y;
a combination of F217A, N268W and L226V;
a combination of F217A, N268W and D239Q;
a combination of F217A, N268W and F546M;
a combination of F217A, N268W and A24N;
a combination of F217A, N268W and A26H;
a combination of F217A, N268W and Y599M;
a combination of F217A, N268W and G157N;
a combination of F217A, N268W and Q45D;
a combination of F217A, N268W and Q45E;
a combination of F217A, N268W and Q45N;
a combination of F217A, N268W and E49K;
a combination of F217A, N268W and N69D;
a combination of F217A, N268W and N69E;
a combination of F217A, N268W and N69F;
a combination of F217A, N268W and N69H;
a combination of F217A, N268W and N69L;
a combination of F217A, N268W and N69M;
a combination of F217A, N268W and N69T;
a combination of F217A, N268W and N69W;
a combination of F217A, N268W and N69Y;
a combination of F217A, N268W and W104G;
a combination of F217A, N268W and W104N;
a combination of F217A, N268W and T175E;
a combination of F217A, N268W and T175H;
a combination of F217A, N268W and T175I;
a combination of F217A, N268W and T175K;
a combination of F217A, N268W and A401D;
a combination of F217A, N268W, A195T and Y352W;
a combination of F217A, N268W, N341W and Y352T;
a combination of F217A, N268W, A198I and N259T;
a combination of F217A, N268W, A195S and N341W;
a combination of F217A, N268W, T153K and A195S;
a combination of F217A, N268W, I28R and K389I;
a combination of F217A, N268W, T153R and A195T;
a combination of F217A, N268W, I28R, T153R, A198I and N341W;
a combination of F217A, N268W, A195T, N259T and N341W;
a combination of F217A, N268W, I28R, A195T, N259T and K389I;
a combination of F217A, N268W, I28R, N259T and N341W;
a combination of F217A, N268W, I28R, A195S and K389I;
a combination of F217A, N268W, A195T and N341W;
a combination of F217A, N268W, I28R, A195T, N341W and K389I;
a combination of F217A, N268W, I28R, A195T, N259T and T447A;
a combination of F217A, N268W, I28R, A195S, N259T and N341W;
a combination of F217A, N268W, I28R, A195T, N259T, N341W and K389I; and
a combination of F217A, N268W, T153R and N341W.

5. The modified galactose oxidase of claim 1, wherein the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence of at least one modified galactose oxidase variant set forth in the even numbered sequences of SEQ ID NOS: 7-298, or the polypeptide sequence of the modified galactose oxidase comprises a polypeptide sequence set forth in the even numbered sequences of SEQ ID NOS: 7-298.

6. (canceled)

7. The modified galactose oxidase of claim 1, wherein
the mutation is selected from the group consisting of:
(a) S314C;
(b) N268W;
(c) F217A;
(d) a combination of F217A and N268W;
(e) a combination of F217A, N268W and A195S;
(f) a combination of F217A, N268W and N259T;
(g) a combination of F217A, N268W and A195T;
(h) a combination of F217A, N268W and A198I;
(i) a combination of F217A, N268W and T202I;
(j) a combination of F217A, N268W and Y352W;
(k) a combination of F217A, N268W and A24H;
(l) a combination of F217A, N268W and I28R;
(m) a combination of F217A, N268W and D239E;
(n) a combination of F217A, N268W and A24N;
(o) a combination of F217A, N268W and E218K;
(p) a combination of F217A, N268W and E218F;
(q) a combination of F217A, N268W and Y599M;
(r) a combination of F217A, N268W and L226Q;
(s) a combination of F217A, N268W and A195V;
(t) a combination of F217A, N268W, A195T and Y352W;
(u) a combination of F217A, N268W, I28R and K389I;
(v) a combination of F217A, N268W, I28R, A195T, N259T, and K389I; and
(w) a combination of F217A, N268W, I28R, A195T, N259T, N341W, and K389I.

8. The modified galactose oxidase of claim 1, wherein the modified galactose oxidase comprises the mutations of F217A and N268W, or the polypeptide sequence of the modified galactose oxidase has at least 85%, 86%, 87%, 88%, 89%, 90%, 91%, 92%, 93%, 94%, 95%, 96%, 97%, 98%, or 99% sequence identity to SEQ ID NO:68.

9. (canceled)

10. The modified galactose oxidase of claim 8, wherein the modified galactose oxidase further comprises at least one mutation selected from the group consisting of A24H, A24N, A26H, A26R, I28F, I28M, I28R, Q45D, Q45E, Q45N, E49K, F65P, N69D, N69E, N69F, N69H, N69L, N69M, N69T, N69W, N69Y, W104G, W104N, S114N, S114R, T117D, T153K, T153R, G157N, S172E, S173K, S173R, T175E, T175H, T175I, T175K, V193T, P194C, A195SX, A195T, A195V, A197C, A197G, A198I, T202D, T202I, T202K, T202Q, N214R, N214S, E218F, E218K, E218P, E218Y, S220T, I224W, L226Q, L226V, D239E, D239P, D239Q, R240G, F250E, F250Y, N259T, T265S, D269N, I284S, I284Y, G294A, F313R, S314C, Y325F, N341W, A346L, K348S, Y352T, Y352W, M353F, N356K, N356R, N356W, K389I, A401 D, T429W, T447A, I485A, I485K, I485R, F487R, L537G, C538K, C538R, F546M, S559P, S576D, Y599M, Q662D, and Q662P, wherein the position of the mutation is numbered with reference to SEQ ID NO: 6; or the polypeptide sequence of the modified galactose oxidase has at least 85%, 86%, 87%, 88%, 89%, 90%, 91%, 92%, 93%, 94%, 95%, 96%, 97%, 98%, or 99% sequence identity to the sequence of at least one modified galactose oxidase variant set forth in SEQ ID NOS: 70, 72 or the even numbered sequences of SEQ ID NOS: 77-298, or the polypeptide sequence of the modified galactose oxidase comprises a polypeptide sequence set forth in SEQ ID NOS: 70, 72 or the even numbered sequences of SEQ ID NOS: 77-298.

11.-12. (canceled)

13. The modified galactose oxidase of claim 1, wherein the modified galactose oxidase comprises one or more of the following properties:
(a) enhanced protein expression by at least 1.01-fold compared to the galactose oxidase variant M3-5 or by at least 1.01-fold compared to a modified galactose oxidase comprising the mutations of F217A and N268W, or the polypeptide sequence of the modified galactose oxidase has at least 85%, 86%, 87%, 88%, 89%, 90%, 91%, 92%, 93%, 94%, 95%, 96%, 97%, 98%, or 99% sequence identity to SEQ ID NO:68;
(b) enhanced solubility by at least 1.01-fold compared to the galactose oxidase variant M3-5 or by at least 1.01-fold compared to a modified galactose oxidase comprising the mutations of F217A and N268W, or the polypeptide sequence of the modified galactose oxidase has at least 85%, 86%, 87%, 88%, 89%, 90%, 91%, 92%, 93%, 94%, 95%, 96%, 97%, 98%, or 99% sequence identity to SEQ ID NO:68;
(c) enhanced thermal stability by at least 1.01-fold compared to the galactose oxidase variant M3-5 or by at least 1.01-fold compared to a modified galactose oxidase of claim 8 at a temperature of at least 50° C. comprising the mutations of F217A and N268W, or the polypeptide sequence of the modified galactose oxidase has at least 85%, 86%, 87%, 88%, 89%, 90%, 91%, 92%, 93%, 94%, 95%, 96%, 97%, 98%, or 99% sequence identity to SEQ ID NO:68;
(d) increased enzymatic activity by at least 1.01-fold compared to the galactose oxidase variant M3-5 or by at least 1.01-fold compared to a modified galactose oxidase comprising the mutations of F217A and N268W, or the polypeptide sequence of the modified galactose oxidase has at least 85%, 86%, 87%, 88%, 89%, 90%, 91%, 92%, 93%, 94%, 95%, 96%, 97%, 98%, or 99% sequence identity to SEQ ID NO:68, wherein the enzymatic activity comprises converting a secondary alcohol to a ketone, wherein optionally the secondary alcohol is a bulky secondary alcohol or unactivated secondary alcohol; and
(e) reduced enantioselectivity by at least 1.01-fold compared to the galactose oxidase variant M3-5 or by at least 1.01-fold compared to a modified galactose oxidase comprising the mutations of F217A and N268W, or the polypeptide sequence of the modified galactose oxidase has at least 85%, 86%, 87%, 88%, 89%, 90%, 91%, 92%, 93%, 94%, 95%, 96%, 97%, 98%, or 99% sequence identity to SEQ ID NO:68.

14.-25. (canceled)

26. A method of producing a ketone from a secondary alcohol, comprising contacting the secondary alcohol with the modified galactose oxidase of claim 1 under suitable conditions for an oxidation reaction.

27. The method of claim 26, wherein:
(i) the secondary alcohol is a bulky secondary alcohol or unactivated secondary alcohol, wherein the bulky secondary alcohol comprises one or more bulky substituents, and wherein the unactivated secondary alcohol is an aliphatic secondary alcohol without a benzene ring in the alpha position adjacent to the alcohol group; or,
(ii) the secondary alcohol comprises the formula R¹R²CHOH, wherein R¹ and R² are independently selected from the group consisting of cycloalkyl, aryl, heteroaryl, alkyl, alkylene, alkynyl, haloalkyl, carboxyl, and carbonyl, wherein the cycloalkyl, aryl, heteroaryl, alkyl, alkylene, alkynyl, haloalkyl, carboxyl, and carbonyl is optionally substituted with one or more substituents selected from the group consisting of alkyl, halogen, alkyl halide, alkylene, alkynyl, alkoxy, cyano, oxo, nitro, amino, thiol, carboxyl, ester and hydroxyl; or,
(iii) the secondary alcohol is selected from the group consisting of: 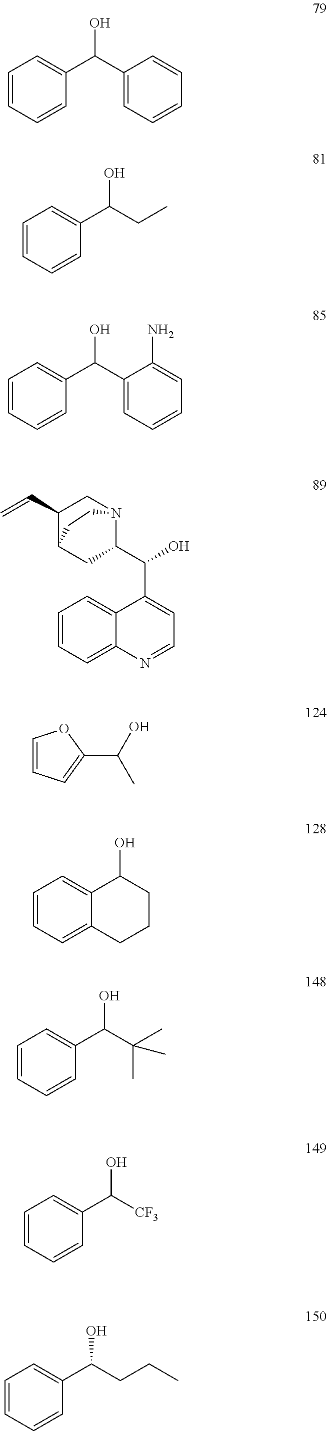 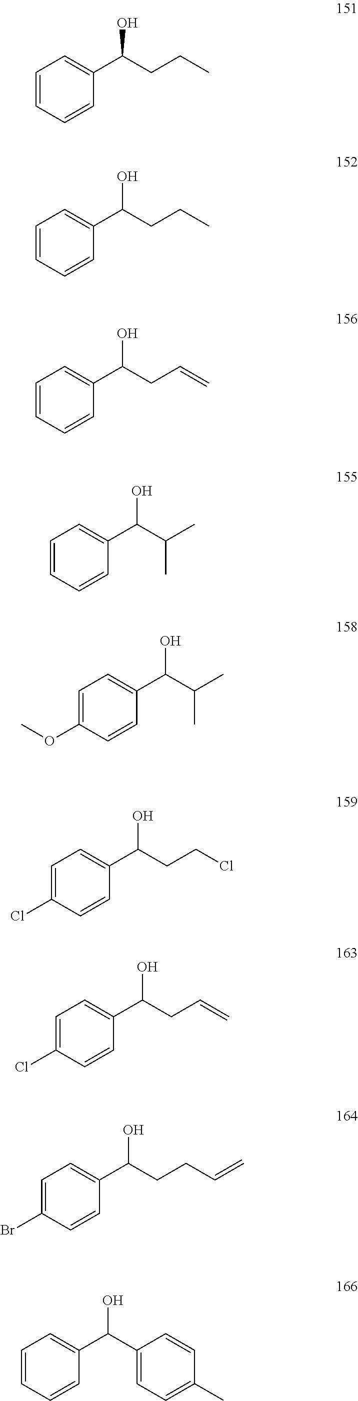 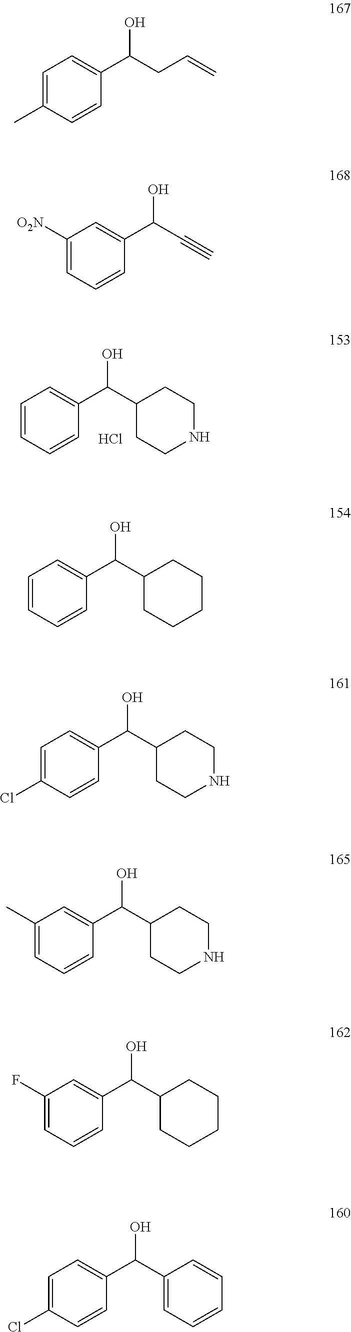 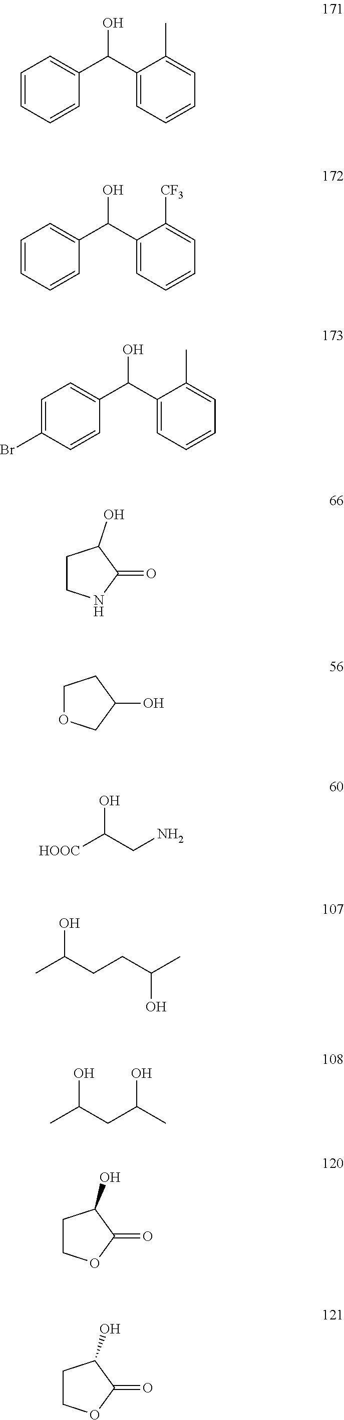 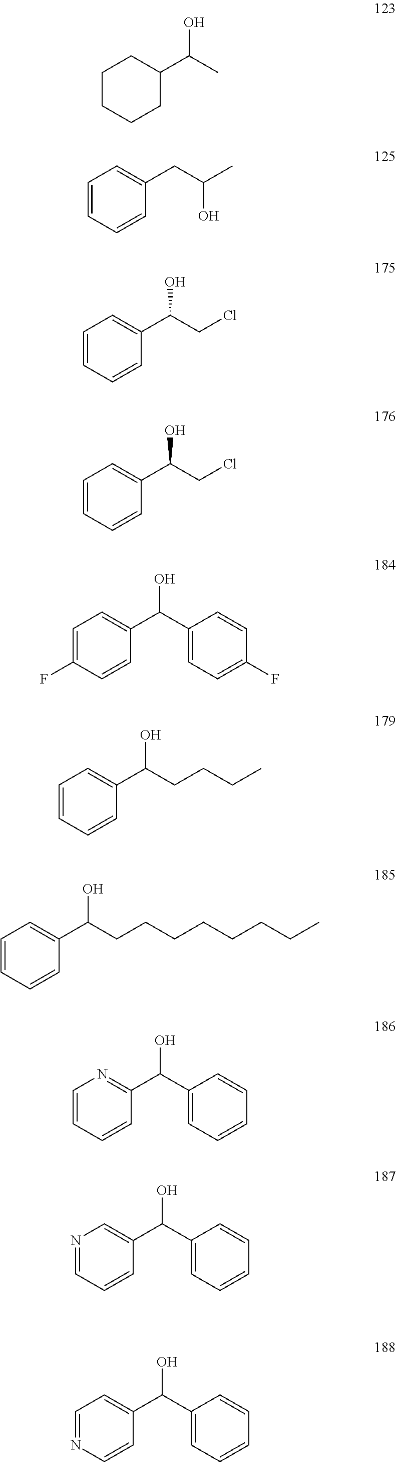 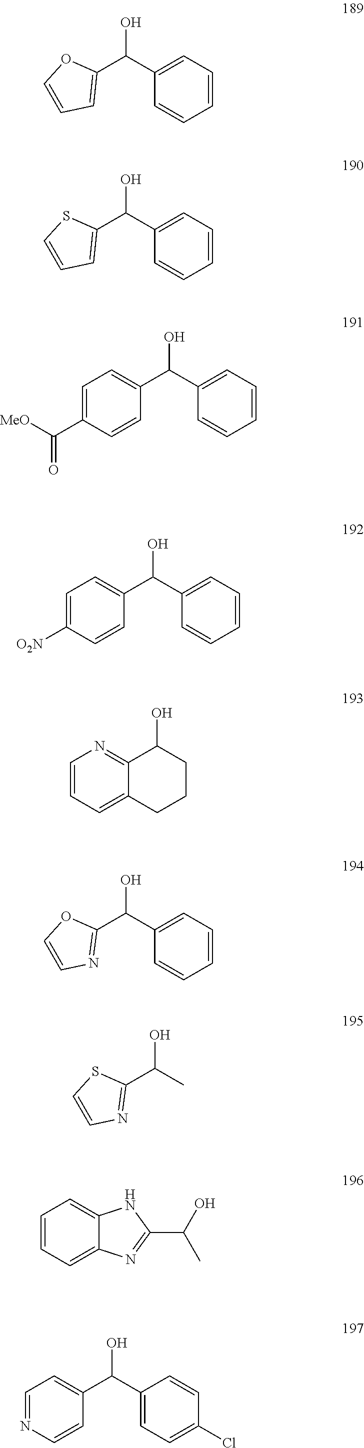 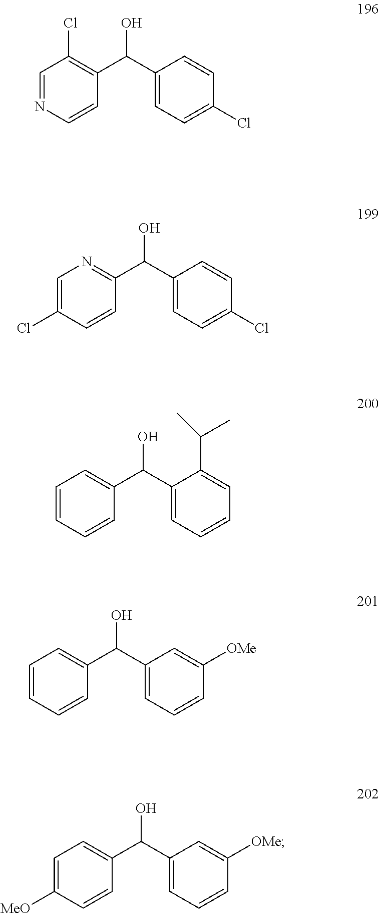 [SMILES: OC(c1ccc(Cl)cc1)c1ccncc1Cl ; OC(c1ccc(Cl)cc1)c1ccc(Cl)cn1 ; CC(C)c1ccccc1C(O)c1ccccc1 ; COc1cccc(C(O)c2ccccc2)c1 ; COc1ccc(C(O)c2cccc(OC)c2)cc1]
or,
(iv) the secondary alcohol is chiral or non-chiral, wherein optionally the chiral secondary alcohol is present as (S)-enantiomer, (R)-enantiomer, or a mixture of (S)- and (R)-enantiomers, wherein optionally the chiral secondary alcohol is present as a racemic mixture.

28.-30. (canceled)

31. The method of claim 26, wherein (i) the contacting step is performed at a temperature of 25° C., 30° C., 35° C., 40° C., 45° C., 50° C., 55° C., or more, wherein optionally the temperature is 35° C.; or (ii), the method further comprises, converting the secondary alcohol into an active pharmaceutical ingredient.

32. (canceled)

33. A kit for use in a method of producing a ketone from a secondary alcohol, comprising contactin the secondary alcohol with the modified galactose oxidase of claim 1 under suitable conditions for an oxidation reaction, wherein the kit comprises the modified galactose oxidase of claim 1.

34. The kit of claim 33, wherein the kit further comprises one or more of the following:
(a) one or more reaction buffers;
(b) one or more reaction vessels, wherein optionally the reaction vessel is a HPLC vial;
(c) one or more reaction solvents, wherein optionally the reaction solvent is 5% v/v dimethyl sulfoxide in 100 mM sodium phosphate buffer with a pH of 7;
(d) one or more secondary alcohol substrates;
(e) one or more co-enzymes to break down the by-product of the oxidation reaction, wherein optionally the co-enzyme is a horseradish peroxidase that breaks down the H₂O₂; and
(f) instructions for performing the method of producing a ketone from a secondary alcohol, comprising contacting the secondary alcohol with the modified galactose oxidase of claim 1 under suitable conditions for an oxidation reaction.
